(19) United States
(12) Patent Application Publication
(10) Pub. No.: US 2021/0230537 A1
(43) Pub. Date: Jul. 29, 2021

(54) ANGIOGENESIS USING STIMULATED PLACENTAL STEM CELLS

(71) Applicant: Celularity Inc., Florham Park, NJ (US)

(72) Inventors: Kathy E. KARASIEWICZ-MENDEZ, Hillsborough, NJ (US); Aleksander FRANCKI, Annandale, NJ (US); Jeffrey TURNER, Summit, NJ (US); Eric LAW, East Brunswick, NJ (US); Jennifer PAREDES, Bloomfield, NJ (US); Kristen LABAZZO, Springfield, IL (US); Hemlata RANA, Clifton, NJ (US); Wolfgang HOFGARTNER, Florham Park, NJ (US); Robert J. HARIRI, Bernardsville, NJ (US)

(73) Assignee: Celularity Inc., Florham Park, NJ (US)

(21) Appl. No.: 17/143,866

(22) Filed: Jan. 7, 2021

Related U.S. Application Data
(63) Continuation of application No. 15/576,810, filed on Nov. 25, 2017, now abandoned.
(60) Provisional application No. 62/166,504, filed on May 26, 2015.

(51) Int. Cl. C12N 5/073; A61K 9/00; A61K 35/50; A61K 35/28; C12N 5/071; A61L 27/38; A61P 9/10

(52) U.S. Cl. CPC C12N 5/0605; A61K 9/0019; A61K 35/50; A61K 9/0085; A61K 35/28; C12N 5/069; A61K 47/36; A61L 27/3895; A61L 27/3834; A61P 9/10; C12N 2501/2301; C12N 2502/025; A61L 27/3808

(57) ABSTRACT

Provided herein are stimulated placental stem cells and methods of treating individuals having diseases or disorders of the circulatory system using stimulated placental cells. The invention also provides methods of inducing angiogenesis using such stimulated cells or populations of cells comprising such stimulated cells.

This application claims benefit of U.S. Provisional Patent Application No. 62/166,504, filed May 26, 2015, the disclosure of which is incorporated by reference herein in its entirety.

### 1. FIELD

Provided herein are methods of using tissue culture plastic-adherent placental cells, e.g. placental stem cells, referred to herein as PDACs, that have been stimulated with one or more cytokines, to promote angiogenesis, and to treat diseases or disorders of the circulatory system, e.g., diseases or disorders associated with or resulting from, inadequate vascularization or blood flow, or treatable by improving angiogenesis.

### 2. BACKGROUND

The placenta is a particularly attractive source of stem cells. Because mammalian placentas are plentiful and are normally discarded as medical waste, they represent a unique source of medically-useful stem cells. Provided herein are such isolated placental stem cells, populations of the placental stem cells, and methods of using the same to promote angiogenesis, and to treat disease or disorders of the circulatory system, e.g., diseases or disorders treatable by promoting angiogenesis.

### 3. SUMMARY

In one aspect, provided herein are tissue culture plastic-adherent placental cells, e.g., placental stem cells, also referred to herein as PDACs (placenta derived adherent cells, e.g., the placenta-derived adherent cells described in Section 5.2, below) that have been stimulated with one or more cytokines. In certain embodiments, the stimulated PDACs are stimulated with pro-inflammatory cytokines. In specific embodiments, the pro-inflammatory cytokines comprise one or more of IL-1 α, IL-1 β, IL-6, IL-8, IL-18, TNF-α, and/or INF-γ. In a specific embodiment, the pro-inflammatory cytokine is IL-1β.

In a specific embodiment, the stimulated PDACs provided herein adhere to tissue culture plastic and are CD34⁻, CD10⁺, CD105⁺, CD200⁺ as determined by, e.g., flow cytometry.

In some embodiments, the stimulated PDACs described herein (e.g., IL-1β-stimulated PDACs) secrete pro-angiogenic factors at a higher level than non-stimulated PDACs (e.g., PDACs that have not been stimulated with a cytokine, e.g., a pro-inflammatory cytokine, e.g., IL-1β). In a specific embodiment, said secreted factors comprise GM-CSF, G-CSF, IL-6, GRO, MCP-1, Follistatin, and/or IL-8. In specific embodiments, said stimulated PDACs described herein (e.g., IL-1β-stimulated PDACs) have pro-angiogenic properties.

In another aspect, provided herein are methods of treating an individual having a disease or disorder of the circulatory system, comprising administering to the individual a therapeutically effective amount of tissue culture plastic-adherent placental cells, e.g., placental stem cells, also referred to herein as PDACs (placenta derived adherent cells, e.g., the placenta-derived adherent cells described in Section 5.2, below) that have been stimulated with one or more cytokines, wherein said stimulated PDACs are administered to the individual in an amount and for a time sufficient for detectable improvement of one or more symptoms of said disease or disorder. In certain embodiments, the stimulated PDACs are stimulated with pro-inflammatory cytokines. In specific embodiments, the pro-inflammatory cytokines comprise one or more of IL-1 α, IL-1 β, IL-6, IL-8, IL-18, TNF-α, and/or INF-γ. In a specific embodiment, the pro-inflammatory cytokine is IL-1β.

In a specific embodiment, provided herein are methods of treating a disease or disorder in a subject in need thereof, comprising administering to the subject a therapeutically effective amount of stimulated PDACs, e.g., IL-1β-stimulated PDACs, e.g., IL-1β-stimulated CD34⁻, CD10⁺, CD105⁺, CD200⁺ PDACs. In a specific embodiment, said stimulated PDACs are formulated as a pharmaceutical composition. In a specific embodiment, said disease or disorder is myocardial infarction. In another specific embodiment, said disease or disorder is congestive heart failure. In another specific embodiment, said disease or disorder is cardiomyopathy.

In another specific embodiment, the disease or disorder treated with stimulated PDACs, e.g., IL-1β-stimulated PDACs, e.g., IL-1β-stimulated CD34⁻, CD10⁺, CD105⁺, CD200⁺ PDACs, is diabetic foot ulcer (DFU). In a specific embodiment, a subject with DFU treated in accordance with the methods provided herein has type I diabetes. In another specific embodiment, a subject with DFU treated in accordance with the methods provided herein has type II diabetes. In certain embodiments, a subject treated in accordance with the methods provided herein has more than one DFU, e.g., the subject has more than one DFU on a single foot, or at least one DFU on each foot. In a specific embodiment, the subject has one or more DFU at the bottom of one foot, or both feet. In certain embodiments, a subject treated in accordance with the methods provided herein has peripheral neuropathy, e.g., damage to one or more of the nerves in the legs and/or feet.

In certain embodiments, a subject with DFU treated in accordance with the methods provided herein has DFU with a condition that causes a disruption in the flow of blood in the subject's peripheral vasculature. In a specific embodiment, the subject has peripheral arterial disease (PAD). In certain embodiments, said DFU is caused by and/or associated with PAD.

In certain embodiments, the methods of treating DFU provided herein result in a detectable improvement of one or more symptoms of DFU in a subject treated in accordance with the methods provided herein. Exemplary symptoms of DFU include, without limitation, sores, ulcers, or blisters on the foot and/or lower leg; pain in the foot (or feet) and/or lower leg; difficulty walking; discoloration in the foot (or feet), e.g., the foot (or feet) appear black, blue, and/or red; and signs of infection (e.g., fever, skin redness, and/or swelling).

In certain embodiments, the methods of treating DFU provided herein comprise administering stimulated PDACs (e.g., a pharmaceutical composition comprising stimulated PDACs) to a subject having DFU in an amount and for a time sufficient for detectable improvement in one or more indicia of improvement, wherein said indicia of improvement include (i) reduction in ulcer size; (ii) ulcer closure: skin closure of one or more ulcers without drainage or the need for dressing; (iii) retention of ulcer closure for a specified time period following closure, e.g., 2 weeks, 3 weeks, 4 weeks, 5 weeks, or 6 weeks following closure; (iv) increased time to ulcer closure; (v) improvement in ankle brachial index (ABI), a test that measures blood pressure at the ankle and in the arm while a subject is at rest and then repeated while a subject is in motion (e.g., walking on a treadmill), and which can be used to predict/assess the severity of PAD; (vi) improvement in toe brachial index (TBI), a test analogous to ABI that uses toe blood pressure as opposed to ankle blood pressure; (vii) improvement in transcutaneous oxygen level, i.e., the oxygen level in the tissue beneath the skin close to the ulcer (see, e.g., Ruangsetakit et al., J Wound Care, 2010, 19(5):202-6); (viii) improvement in pulse volume recording, which is a noninvasive vascular test in which blood pressure cuffs and a hand-held ultrasound device are used to obtain information about arterial blood flow in the arms and legs; (ix) time to major amputation, e.g., amputation above the ankle; (x) improvement on the Wagner Grading Scale, which assesses ulcer depth and the presence of osteomyelitis or gangrene using a grading system: grade 0 (pre- or post-ulcerative lesion), grade 1 (partial/full thickness ulcer), grade 2 (probing to tendon or capsule), grade 3 (deep with osteitis), grade 4 (partial foot gangrene), and grade 5 (whole foot gangrene); (xi) improvement in Rutherford criteria, which is used for staging of peripheral arterial disease has seven classification stages: Stage 0—Asymptomatic, Stage 1—mild claudication, Stage 2—moderate claudication, Stage 3—severe claudication, Stage 4—rest pain, Stage 5—ischemic ulceration not exceeding ulcer of the digits of the foot, and Stage 6—severe ischemic ulcers or frank gangrene; and (xii) improvement in leg rest pain score, a β-10 scale of pain with 0 being pain free and 10 representing maximum pain.

In certain embodiments, the methods of treating DFU provided herein comprise administering placental stem cells (e.g., a pharmaceutical composition comprising placental stem cells) to a subject having DFU in an amount and for a time sufficient for detectable improvement in quality of life of the subject as assessed by, e.g., (i) a 36-item Short Form Health Survey (SF-36) (see, e.g., Ware et al., Medical Care 30(6):473-483); (ii) the Diabetic Foot Ulcer Scale Short Form (DFS-SF), which measures the impact of diabetic foot ulcer on quality of life (see, e.g., Bann et al., Pharmacoeconomics, 2003, 21(17):1277-90); (iii) the Patient Global Impression of Change Scale, to assess changes in neuropathy over time (see, e.g., Kamper et al., J. Man. Manip. Ther., 2009, 17(3):163-170); and/or (iv) the EuroQol5D (EQ-SD™) Scale, which is a health questionnaire used to obtain a descriptive profile and single index value for health status of a patient.

In other embodiments, the disease or disorder treated with stimulated PDACs, e.g., IL-1β-stimulated PDACs, e.g., IL-1β-stimulated CD34⁻, CD10⁺, CD105⁺, CD200⁺ PDACs, is aneurysm, angina, aortic stenosis, aortitis, arrhythmias, arteriosclerosis, arteritis, asymmetric septal hypertrophy (ASH), atherosclerosis, atrial fibrillation and flutter, bacterial endocarditis, Barlow's Syndrome (mitral valve prolapse), bradycardia, Buerger's Disease (thromboangiitis obliterans), cardiomegaly, carditis, carotid artery disease, coarctation of the aorta, congenital heart defects, coronary artery disease, Eisenmenger's Syndrome, embolism, endocarditis, erythromelalgia, fibrillation, fibromuscular dysplasia, heart block, heart murmur, hypertension, hypotension, idiopathic infantile arterial calcification, Kawasaki Disease (mucocutaneous lymph node syndrome, mucocutaneous lymph node disease, infantile polyarteritis), metabolic syndrome, microvascular angina, myocarditis, paroxysmal atrial tachycardia (PAT), periarteritis nodosa (polyarteritis, polyarteritis nodosa), pericarditis, peripheral vascular disease, critical limb ischemia, phlebitis, pulmonary valve stenosis (pulmonic stenosis), Raynaud's Disease, renal artery stenosis, renovascular hypertension, rheumatic heart disease, diabetic vasculopathy, septal defects, silent ischemia, syndrome X, tachycardia, Takayasu's Arteritis, Tetralogy of Fallot, transposition of the great vessels, tricuspid atresia, truncus arteriosus, valvular heart disease, varicose ulcers, varicose veins, vasculitis, ventricular septal defect, Wolff-Parkinson-White Syndrome, endocardial cushion defect, acute rheumatic fever, acute rheumatic pericarditis, acute rheumatic endocarditis, acute rheumatic myocarditis, chronic rheumatic heart diseases, diseases of the mitral valve, mitral stenosis, rheumatic mitral insufficiency, diseases of aortic valve, diseases of other endocardial structures, ischemic heart disease (acute and subacute), angina pectoris, acute pulmonary heart disease, pulmonary embolism, chronic pulmonary heart disease, kyphoscoliotic heart disease, myocarditis, endocarditis, endomyocardial fibrosis, endocardial fibroelastosis, atrioventricular block, cardiac dysrhythmias, myocardial degeneration, cerebrovascular disease, a disease of arteries, arterioles and capillaries, or a disease of veins and lymphatic vessels.

In other specific embodiments, the disease or disorder treated with stimulated PDACs, e.g., IL-1β-stimulated PDACs, e.g., IL-1β-stimulated CD34⁻, CD10⁺, CD105⁺, CD200⁺ PDACs, is an occlusion and stenosis of precerebral arteries, or occlusion of cerebral arteries. In one embodiment, provided herein is a method of treating an individual who has a disruption of the flow of blood in or around the individual's brain, e.g., who has a symptom or neurological deficit attributable to a disruption of the flow of blood in or around the individual's brain or central nervous system (CNS), comprising administering to said individual a therapeutically effective amount of stimulated PDACs (e.g., IL-1 β-stimulated PDACs). In certain embodiments, the disruption of flow of blood results in anoxic injury or hypoxic injury to the individual's brain or CNS.

In other specific embodiments, the disease or disorder treated with stimulated PDACs, e.g., IL-1β-stimulated PDACs, e.g., IL-1β-stimulated CD34⁻, CD10⁺, CD105⁺, CD200⁺ PDACs, is an occlusion and stenosis of peripheral arteries. In one embodiment, provided herein is a method of treating an individual who has a disruption of the flow of blood in or around limb, e.g., who has a symptom or vascular deficit attributable to a disruption of the flow of blood in or around the individual's peripheral vascular system, comprising administering to said individual a therapeutically effective amount of stimulated PDACs (e.g., IL-1 β-stimulated PDACs). In certain embodiments, the disruption of flow of blood results in anoxic injury or hypoxic injury to the individual's limbs and or extremities.

In a specific embodiment of the methods of treatment described herein, the stimulated PDACs (e.g., a pharmaceutical composition comprising stimulated PDACs) are administered by injection. In another specific embodiment of the methods of treatment described herein, the stimulated PDACs (e.g., a pharmaceutical composition comprising stimulated PDACs) are administered to a subject being treated by implantation in said subject of a matrix or scaffold comprising placental cells.

In a specific embodiment of the methods of treatment described herein, the stimulated PDACs (e.g., a pharmaceutical composition comprising stimulated PDACs) are administered intramuscularly. In another specific embodiment of the methods of treatment described herein, the stimulated PDACs (e.g., a pharmaceutical composition comprising stimulated PDACs) are administered intravenously. In another specific embodiment of the methods of treatment described herein, the stimulated PDACs (e.g., a pharmaceutical composition comprising stimulated PDACs) are administered subcutaneously. In another specific embodiment of the methods of treatment described herein, the stimulated PDACs (e.g., a pharmaceutical composition comprising stimulated PDACs) are administered locally. In another specific embodiment of the methods of treatment described herein, the stimulated PDACs (e.g., a pharmaceutical composition comprising stimulated PDACs) are administered systemically. In another specific embodiment of the methods of treatment described herein, the stimulated PDACs (e.g., a pharmaceutical composition comprising stimulated PDACs) are administered directly to the site of the disease being treated, e.g., an ulcer, e.g., a diabetic foot ulcer. In another specific embodiment of the methods of treatment described herein, the stimulated PDACs (e.g., a pharmaceutical composition comprising stimulated PDACs) are administered adjacent or peripheral to the site of the disease being treated, e.g., an ulcer, e.g., a diabetic foot ulcer.

In certain embodiments, the methods of treatment described herein comprise administration of about 1×10⁶, 3×10⁶, 5×10⁶, 1×10⁷, 3×10⁷, 5×10⁷, 1×10⁸, 3×10⁸, 5×10⁸, 1×10⁹, 5×10⁹, or 1×10¹⁰ stimulated PDACs (e.g., as part of a pharmaceutical composition comprising stimulated PDACs). In certain embodiments, the methods of treatment described herein comprise administration of about 1×10⁶ to 3×10⁶, 3×10⁶ to 5×10⁶, 5×10⁶ to 1×10⁷, 1×10⁷ to 3×10⁷, 3×10⁷ to 5×10⁷, 5×10⁷ to 1×10⁸, 1×10⁸ to 3×10⁸, 3×10⁸ to 5×10⁸, 5×10⁸ to 1×10⁹, 1×10⁹ to 5×10⁹, or 5×10⁹ to 1×10¹⁰ stimulated PDACs (e.g., as part of a pharmaceutical composition comprising stimulated PDACs). In a specific embodiment, the methods of treatment described herein comprise administration of about 3×10⁶ stimulated PDACs. In another specific embodiment, the methods of treatment described herein comprise administration of about 1×10⁷ stimulated PDACs. In another specific embodiment, the methods of treatment described herein comprise administration of about 3×10⁷ stimulated PDACs. In another specific embodiment, the methods of treatment described herein comprise administration of about 1×10⁸ stimulated PDACs.

In a specific embodiment of the methods of treatment described herein, the stimulated PDACs (e.g., a pharmaceutical composition comprising stimulated PDACs) are administered intramuscularly to a subject more than once, with one week between administrations, e.g., stimulated PDACs are administered on day 1 (the first day of administration) and a second dose of stimulated PDACs (e.g., a pharmaceutical composition comprising stimulated PDACs) is administered one week later (i.e., on day 8). In another specific embodiment, the methods comprise administration of about 3×10⁶ stimulated PDACs (e.g., a pharmaceutical composition comprising stimulated PDACs) on each day of administration (i.e., on days 1 and 8). In another specific embodiment, the methods comprise administration of about 1×10⁷ stimulated PDACs (e.g., a pharmaceutical composition comprising stimulated PDACs) on each day of administration (i.e., on days 1 and 8). In another specific embodiment, the methods comprise administration of about 3×10⁷ stimulated PDACs (e.g., a pharmaceutical composition comprising stimulated PDACs) on each day of administration (i.e., on days 1 and 8). In another specific embodiment, the methods comprise administration of about 1×10⁸ stimulated PDACs (e.g., a pharmaceutical composition comprising stimulated PDACs) on each day of administration (i.e., on days 1 and 8). In another specific embodiment, the stimulated PDACs (e.g., a pharmaceutical composition comprising stimulated PDACs) are administered to a subject on at least three different occasions, with about one week between administrations.

In another specific embodiment of the methods of treatment described herein, the stimulated PDACs (e.g., a pharmaceutical composition comprising stimulated PDACs) are administered to a subject more than once, with one month between administrations, e.g., stimulated PDACs are administered on day 1 (the first day of administration) and a second dose of stimulated PDACs (e.g., a pharmaceutical composition comprising stimulated PDACs) is administered about one month later (e.g., on day 27, 28, 29, 30, 31, 32, or 33). In a specific embodiment, the methods comprise administration of about 3×10⁶ stimulated PDACs (e.g., a pharmaceutical composition comprising stimulated PDACs) on each day of administration (e.g., 3×10⁶ stimulated PDACs are administered on day 1, and about 3×10⁶ stimulated PDACs (e.g., a pharmaceutical composition comprising stimulated PDACs) are administered 1 month after day 1, e.g., on day 27, 28, 29, 30, 31, 32, or 33). In another specific embodiment, the methods comprise administration of about 3×10⁷ stimulated PDACs (e.g., a pharmaceutical composition comprising stimulated PDACs) on each day of administration (e.g., 3×10⁷ stimulated PDACs are administered on day 1, and about 3×10⁷ stimulated PDACs are administered 1 month after day 1, e.g., on day 27, 28, 29, 30, 31, 32, or 33). In another specific embodiment, the methods comprise administration of about 1×10⁸ stimulated PDACs (e.g., a pharmaceutical composition comprising stimulated PDACs) on each day of administration (e.g., 1×10⁸ stimulated PDACs are administered on day 1, and about 1×10⁸ stimulated PDACs are administered 1 month after day 1, e.g., on day 27, 28, 29, 30, 31, 32, or 33). In another specific embodiment, the stimulated PDACs (e.g., a pharmaceutical composition comprising stimulated PDACs) are administered are administered to a subject on at least three different occasions, with about one month between administrations.

In various embodiments, the stimulated PDACs useful in the methods disclosed herein are contained within a population of cells, at least 10%, 15%, 20%, 25%, 30%, 35%, 40%, 45%, 50%, 55%, 60%, 65%, 70%, 75%, 80%, 85%, 90%, 95%, 98% or 99% of the cells of which are said stimulated PDACs. In certain embodiments, the stimulated PDACs in said population of cells are substantially free of cells having a maternal genotype; e.g., at least 40%, 45%, 50%, 55%, 60%, 65%, 70%, 75%, 80%, 85%, 90%, 95%, 98% or 99% of the stimulated PDACs in said population have a fetal genotype, i.e., are fetal in origin. In certain embodiments, the population of cells comprising said stimulated PDACs comprises cells having a maternal genotype; e.g., at least 10%, 15%, 20%, 25%, 30%, 35%, 40%, 45%, 50%, 55%, 60%, 65%, 70%, 75%, 80%, 85%, 90%, 95%, 98% or 99% of the cells in said population have a maternal genotype, i.e., are maternal in origin.

In certain embodiments, the stimulated PDACs used in the methods described herein are autologous to a recipient. In certain embodiments, the stimulated PDACs used in the methods described herein are heterologous to a recipient.

In certain embodiments, the stimulated PDACs used in the methods described herein are cryopreserved prior to administration to a subject. In certain embodiments, the stimulated PDACs used in the methods described herein are obtained from a placental cell bank (e.g., a PDAC bank).

### 3.1 Definitions

As used herein, the term “about,” when referring to a stated numeric value, indicates a value within plus or minus 10% of the stated numeric value.

As used herein, the term “angiogenic,” in reference to the placental derived adherent cells described herein, means that the cells can form vessels or vessel-like sprouts, or that the cells can promote angiogenesis (e.g., the formation of vessels or vessel-like structures) in another population of cells, e.g., endothelial cells.

As used herein, the term “angiogenesis” refers to the process of blood vessel formation that includes, but is not limited to, endothelial cell activation, migration, proliferation, matrix remodeling and cell stabilization.

As used herein, the term “derived” means isolated from or otherwise purified. For example, placental derived adherent cells are isolated from placenta. The term “derived” encompasses cells that are cultured from cells isolated directly from a tissue, e.g., the placenta, and cells cultured or expanded from primary isolates.

As used herein, “immunolocalization” means the detection of a compound, e.g., a cellular marker, using an immune protein, e.g., an antibody or fragment thereof in, for example, flow cytometry, fluorescence-activated cell sorting, magnetic cell sorting, in situ hybridization, immunohistochemistry, or the like.

As used herein, the term “SH2” refers to an antibody that binds an epitope on the cellular marker CD105. Thus, cells that are referred to as SH2⁺ are CD105⁺.

As used herein, the terms “SH3” and SH4⁺ refer to antibodies that bind epitopes present on the cellular marker CD73. Thus, cells that are referred to as SH3⁺ and/or SH4⁺ are CD73⁺.

A placenta has the genotype of the fetus that develops within it, but is also in close physical contact with maternal tissues during gestation. As such, as used herein, the term “fetal genotype” means the genotype of the fetus, e.g., the genotype of the fetus associated with the placenta from which particular isolated placental cells, as described herein, are obtained, as opposed to the genotype of the mother that carried the fetus. As used herein, the term “maternal genotype” means the genotype of the mother that carried the fetus, e.g., the fetus associated with the placenta from which particular isolated placental cells, as described herein, are obtained.

As used herein, the term “isolated cell,” e.g., “isolated placental cell,” “isolated placental stem cell,” and the like, means a cell that is substantially separated from other, different cells of the tissue, e.g., placenta, from which the stem cell is derived. A cell is “isolated” if at least 50%, 60%, 70%, 80%, 90%, 95%, or at least 99% of the cells, e.g., non-stem cells, with which the stem cell is naturally associated, or stem cells displaying a different marker profile, are removed from the stem cell, e.g., during collection and/or culture of the stem cell.

As used herein, “multipotent,” when referring to a cell, means that the cell has the ability to differentiate into some, but not necessarily all, types of cells of the body, or into cells having characteristics of some, but not all, types of cells of the body, or into cells of one or more of the three germ layers. In certain embodiments, for example, an isolated placental cell (PDAC), as described in Section 5.2, below, that has the capacity to differentiate into a cell having characteristics of neurogenic, chondrogenic and/or osteogenic cells is a multipotent cell.

As used herein, the term “population of isolated cells” means a population of cells that is substantially separated from other cells of a tissue, e.g., placenta, from which the population of cells is derived.

As used herein, the term “placental cell” refers to a stem cell or progenitor cell that is isolated from a mammalian placenta, e.g., as described in Section 5.2, below, or cultured from cells isolated from a mammalian placenta, also referred to herein as “PDACs,” either as a primary isolate or a cultured cell, regardless of the number of passages after a primary culture. In certain embodiments, the term “placental cells” as used herein does not, however, refer to, and the placental cells used in the methods provided herein are not, however, trophoblasts, cytotrophoblasts, syncitiotrophoblasts, angioblasts, hemangioblasts, embryonic germ cells, embryonic stem cells, cells obtained from an inner cell mass of a blastocyst, or cells obtained from a gonadal ridge of a late embryo, e.g., an embryonic germ cell. The placental cells, e.g., PDACs, described herein are not the amnion-derived adherent cells described in pending U.S. patent application Ser. No. 12/622,352, filed Nov. 19, 2009, entitled “Amnion Derived Angiogenic Cells,” the disclosure of which is hereby incorporated by reference in its entirety. A cell is considered a “stem cell” if the cell displays attributes of a stem cell, e.g., a marker or gene expression profile associated with one or more types of stem cells; the ability to replicate at least 10-40 times in culture, and the ability to differentiate into cells displaying characteristics of differentiated cells of one or more of the three germ layers. Unless otherwise noted herein, the term “placental” includes the umbilical cord. The isolated placental cells disclosed herein, in certain embodiments, differentiate in vitro under differentiating conditions, differentiate in vivo, or both.

As used herein, a placental cell is “positive” for a particular marker when that marker is detectable above background. Detection of a particular marker can, for example, be accomplished either by use of antibodies, or by oligonucleotide probes or primers based on the sequence of the gene or mRNA encoding the marker. For example, a placental cell is positive for, e.g., CD73 because CD73 is detectable on placental cells in an amount detectably greater than background (in comparison to, e.g., an isotype control). A cell is also positive for a marker when that marker can be used to distinguish the cell from at least one other cell type, or can be used to select or isolate the cell when present or expressed by the cell. In the context of, e.g., antibody-mediated detection, “positive,” as an indication a particular cell surface marker is present, means that the marker is detectable using an antibody, e.g., a fluorescently-labeled antibody, specific for that marker; “positive” also refers to a cell exhibiting the marker in an amount that produces a signal, e.g., in a cytometer, that is detectably above background. For example, a cell is “CD200⁺” where the cell is detectably labeled with an antibody specific to CD200, and the signal from the antibody is detectably higher than that of a control (e.g., background or an isotype control). Conversely, “negative” in the same context means that the cell surface marker is not detectable using an antibody specific for that marker compared a control (e.g., background or an isotype control). For example, a cell is “CD34” where the cell is not reproducibly detectably labeled with an antibody specific to CD34 to a greater degree than a control (e.g., background or an isotype control). Markers not detected, or not detectable, using antibodies are determined to be positive or negative in a similar manner, using an appropriate control. For example, a cell or population of cells can be determined to be OCT-4⁺ if the amount of OCT-4 RNA detected in RNA from the cell or population of cells is detectably greater than background as determined, e.g., by a method of detecting RNA such as RT-PCR, slot blots, etc. Unless otherwise noted herein, cluster of differentiation (“CD”) markers are detected using antibodies. In certain embodiments, OCT-4 is determined to be present, and a cell is “OCT-4⁺” if OCT-4 is detectable using RT-PCR.

As used herein, the designation “low,” when referring to the expression of a marker detectable in flow cytometry, means that the marker is expressed by fewer than 10% of cells tested, or that fluorescence attributable to the marker in, e.g., flow cytometry, is less than 1 log above background.

As used herein, “treat” encompasses the remediation of, improvement of, lessening of the severity of, or reduction in the time course of, a disease, disorder or condition, or any parameter or symptom thereof.

As used herein, “stimulated,” when used in the context of a stimulated placental stem cell (e.g., a stimulated PDAC) refers to a cell that has been contacted with one or more molecules that alter the phenotype of the contacted cell. For example, placental stem cells may be contacted with one or more cytokines that alters the cells in some manner. The changes observed in a stimulated placental PDAC may encompass, e.g., changes in gene expression, secretion of soluble factors, or rates of growth and/or cell division. In a specific embodiment, a stimulated PDAC (e.g., a PDAC that has been stimulated with a cytokine, e.g., a pro-inflammatory cytokine, e.g., IL-1β) secretes pro-angiogenic factors at a higher level than a non-stimulated PDAC (e.g., a PDAC that has not been stimulated with a cytokine, e.g., a pro-inflammatory cytokine, e.g., IL-1β) under the same or similar experimental conditions. In a specific embodiment, said secreted pro-angiogenic factors comprise GM-CSF, G-CSF, IL-6, GRO, MCP-1, Follistatin, and/or IL-8. In specific embodiments, said stimulated PDACs described herein (e.g., IL-1β-stimulated PDACs) have pro-angiogenic properties.

### 4. BRIEF DESCRIPTION OF THE FIGURES

FIG. 1 shows levels of certain growth factors and cytokines secreted by plastic-adherent placental cells (PDACs).

FIGS. 2A-2C show effects of PDAC cell-conditioned media (P-CM) on the growth and survival (FIG. 2A), cellular network length (FIG. 2B) and number of cellular tubes formed (FIG. 2C) for human vascular endothelial cells (HUVECs)

FIG. 3 shows time-dependent effects of P-CM on multiple phosphorylation signaling pathways in cultured HUVECs.

FIG. 4 shows effects of P-CM treatment on HUVEC gene expression over 48 hours.

FIG. 5 shows effects of IL-10 on various factors secreted by PDACs.

FIG. 6 shows effects of P-CM isolated from IL-1β-stimulated PDACs on HUVEC cell signaling pathways.

FIGS. 7A-D show effects of Hepatic Growth Factor (HGF) blockade during treatment of HUVECs with P-CM isolated from IL-1β-stimulated PDACs for the MEK (FIG. 7A), ERK1/2 (FIG. 7B), STAT3 (FIG. 7C), and Akt (FIG. 7D) pathways.

### 5. DETAILED DESCRIPTION

### 5.1 Angiogenesis and Treatment of Diseases or Conditions Associated with or Resulting from Poor Blood Flow

Provided herein are methods of treating diseases or disorders of the circulatory system comprising administering an effective amount of tissue culture plastic-adherent placental cells, e.g., PDACs, as described in Section 5.2, below, wherein said PDACs have been stimulated with a cytokine. In specific embodiments, said PDACs are stimulated with one or more pro-inflammatory cytokines. In a specific embodiment, said pro-inflammatory cytokines comprise one or more of IL-1 α, IL-1 β, IL-6, IL-8, IL-18, TNF-α, and INF-γ. In another specific embodiment, said pro-inflammatory cytokine is IL-1β. In certain embodiments, the stimulated PDACs are angiogenic.

### 5.1.1 Circulatory System Diseases

The stimulated PDACs, e.g., IL-1 β-stimulated PDACs, and populations of such cells, provided herein can be used to treat individuals exhibiting a variety of disease states or conditions that would benefit from increased angiogenesis. Examples of such disease states or conditions include myocardial infarction, peripheral artery disease, hypoplastic left heart syndrome, diabetic ulcer, decubitus ulcer, venous ulcer, arterial ulcer, burn, non-union fracture, osteoarthritis and maxillofacial bone repair. The stimulated PDACs, and populations of such cells, can, in certain embodiments, be used to promote angiogenesis in individuals exhibiting traumatic tissue loss, or to prevent scar formation, or in individuals having total joint replacement or dental prosthetics. In a specific embodiment, said disease or disorder is congestive heart failure.

In a specific embodiment, the stimulated PDACs, e.g., IL-1 β-stimulated PDACs, and populations of such cells, provided herein, can be used to treat an individual having an insufficiency of the circulatory system, e.g., and individual having peripheral vascular disease or coronary artery disease.

In one aspect, provided herein are methods for treating a patient with a heart disease or injury comprising administering a therapeutic cell composition to a patient with a disease or injury of the heart or circulatory system, and evaluating the patient for improvements in cardiac function, wherein said cell composition comprises stimulated PDACs (e.g., IL-1β-stimulated PDACs) as described herein. In one embodiment, the heart disease is a cardiomyopathy. In specific embodiments, the cardiomyopathy is either idiopathic or a cardiomyopathy with a known cause. In other specific embodiments, the cardiomyopathy is either ischemic or nonischemic in nature. In another embodiments, the disease of the heart or circulatory system comprises one or more of angioplasty, aneurysm, angina (angina pectoris), aortic stenosis, aortitis, arrhythmias, arteriosclerosis, arteritis, asymmetric septal hypertrophy (ASH), atherosclerosis, atrial fibrillation and flutter, bacterial endocarditis, Barlow's Syndrome (mitral valve prolapse), bradycardia, Buerger's Disease (thromboangiitis obliterans), cardiomegaly, cardiomyopathy, carditis, carotid artery disease, coarctation of the aorta, congenital heart diseases (congenital heart defects), coronary artery disease, Eisenmenger's Syndrome, embolism, endocarditis, erythromelalgia, fibrillation, fibromuscular dysplasia, heart block, heart murmur, hypertension, hypotension, idiopathic infantile arterial calcification, Kawasaki Disease (mucocutaneous lymph node syndrome, mucocutaneous lymph node disease, infantile polyarteritis), metabolic syndrome, microvascular angina, myocardial infarction (heart attacks), myocarditis, paroxysmal atrial tachycardia (PAT), periarteritis nodosa (polyarteritis, polyarteritis nodosa), pericarditis, peripheral vascular disease, critical limb ischemia, diabetic vasculopathy, phlebitis, pulmonary valve stenosis (pulmonic stenosis), Raynaud's Disease, renal artery stenosis, renovascular hypertension, rheumatic heart disease, septal defects, silent ischemia, syndrome X, tachycardia, Takayasu's Arteritis, Tetralogy of Fallot, transposition of the great vessels, tricuspid atresia, truncus arteriosus, valvular heart disease, varicose ulcers, varicose veins, vasculitis, ventricular septal defect, Wolff-Parkinson-White Syndrome, or endocardial cushion defect. In a specific embodiment, said disease or disorder is heart failure, e.g., congestive heart failure.

In other embodiments, the disease of the heart or circulatory system comprises one or more of acute rheumatic fever, acute rheumatic pericarditis, acute rheumatic endocarditis, acute rheumatic myocarditis, chronic rheumatic heart diseases, diseases of the mitral valve, mitral stenosis, rheumatic mitral insufficiency, diseases of aortic valve, diseases of other endocardial structures, ischemic heart disease (acute and subacute), angina pectoris, diseases of pulmonary circulation (acute pulmonary heart disease, pulmonary embolism, chronic pulmonary heart disease), kyphoscoliotic heart disease, myocarditis, endocarditis, endomyocardial fibrosis, endocardial fibroelastosis, atrioventricular block, cardiac dysrhythmias, myocardial degeneration, diseases of the circulatory system including cerebrovascular disease, occlusion and stenosis of precerebral arteries, occlusion of cerebral arteries, diseases of arteries, arterioles and capillaries (atherosclerosis, aneurysm), or diseases of veins and lymphatic vessels In another specific embodiment, said disease or disorder is diabetic foot ulcer.

In another embodiment, treatment comprises treatment of an individual with a cardiomyopathy with a therapeutic cell composition comprising stimulated PDACs, e.g., IL-1 β-stimulated PDACs, either with or without another cell type. In certain embodiments, the individual experiences benefits from the therapy, for example from the ability of the cells to support the growth of other cells, including stem cells or progenitor cells present in the heart, from the tissue ingrowth or vascularization of the tissue, and from the presence of beneficial cellular factors, chemokines, cytokines and the like, but the cells do not integrate or multiply in the individual. In another embodiment, the patient benefits from the therapeutic treatment with the cells, but the cells do not survive for a prolonged period in the patient. In one embodiment, the cells gradually decline in number, viability or biochemical activity, in other embodiments, the decline in cells may be preceded by a period of activity, for example growth, division, or biochemical activity. In other embodiments, senescent, nonviable or even dead cells are able to have a beneficial therapeutic effect.

Improvement in an individual having a disease or disorder of the circulatory system, wherein the individual is administered the stimulated PDACs or therapeutic compositions provided herein, can be assessed or demonstrated by detectable improvement in one or more symptoms of the disease or disorder of the circulatory system.

In another embodiment, improvement in an individual having a disease or disorder of the circulatory system, wherein the individual is administered the stimulated PDACs or therapeutic compositions comprising the stimulated PDACs, can be assessed or demonstrated by detectable improvement in one or more, indicia of cardiac function, for example, demonstration of detectable improvement in one or more of chest cardiac output (CO), cardiac index (CI), pulmonary artery wedge pressures (PAWP), and cardiac index (CI), % fractional shortening (% FS), ejection fraction (EF), left ventricular ejection fraction (LVEF); left ventricular end diastolic diameter (LVEDD), left ventricular end systolic diameter (LVESD), contractility (e.g. dP/dt), pressure-volume loops, measurements of cardiac work, an increase in atrial or ventricular functioning; an increase in pumping efficiency, a decrease in the rate of loss of pumping efficiency, a decrease in loss of hemodynamic functioning; and a decrease in complications associated with cardiomyopathy, as compared to the individual prior to administration of stimulated PDACs.

Improvement in an individual receiving the stimulated PDACs, e.g., IL-1 β-stimulated PDACs, or therapeutic compositions comprising stimulated PDACs, provided herein can also be assessed by subjective metrics, e.g., the individual's self-assessment about his or her state of health following administration.

Success of administration of the cells is not, in certain embodiments, based on survival in the individual of the administered stimulated PDACs, e.g., IL-1 β-stimulated PDACs. Success is, instead, based on one or more metrics of improvement in cardiac or circulatory health, as noted above. Thus, the cells need not integrate and beat with the patient's heart, or into blood vessels.

Administration of stimulated PDACs, e.g., IL-1 β-stimulated PDACs, or therapeutic compositions comprising such cells, to an individual in need thereof, can be accomplished, e.g., by transplantation, implantation (e.g., of the cells themselves or the cells as part of a matrix-cell combination), injection (e.g., directly to the site of the disease or condition, for example, directly to an ischemic site in the heart of an individual who has had a myocardial infarction), infusion, delivery via catheter, or any other means known in the art for providing cell therapy.

In one embodiment, the therapeutic cell compositions are provided to an individual in need thereof, for example, by injection into one or more sites in the individual. In a specific embodiment, the therapeutic cell compositions are provided by intracardiac injection, e.g., to an ischemic area in the heart. In other specific embodiments, the cells are injected onto the surface of the heart, into an adjacent area, or even to a more remote area. In preferred embodiments, the cells can home to the diseased or injured area.

An individual having a disease or condition of the coronary or vascular system can be administered stimulated PDACs at any time the cells would be therapeutically beneficial. In certain embodiments, for example, the stimulated PDACs, e.g., IL-1 β-stimulated PDACs or therapeutic compositions of the invention are administered within 1, 2, 3, 4, 5, 6, 7, 8, 9, 10, 11, 12, 13, 14, 15, 16, 17, 18, 19, 20, 21, 22, 23, or 24 hours, or 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of the myocardial infarction. Administration proximal in time to a myocardial infarction, e.g., within 1-3 or 1-7 days, is preferable to administration distal in time, e.g., after 3 or 7 days after a myocardial infarction. In other embodiments, the cells or therapeutic compositions of the invention are administered within 1, 2, 3, 4, 5, 6, 7, 8, 9, 10, 11, 12, 13, 14, 15, 16, 17, 18, 19, 20, 21, 22, 23, or 24 hours, or 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of initial diagnosis of the disease or condition.

Also provided herein are kits for use in the treatment of myocardial infarction. The kits provide a therapeutic cell composition comprising stimulated PDACs, e.g., IL-1 β-stimulated PDACs, which can be prepared in a pharmaceutically acceptable form, for example by mixing with a pharmaceutically acceptable carrier, and an applicator, along with instructions for use. Ideally the kit can be used in the field, for example in a physician's office, or by an emergency care provider to be applied to a patient diagnosed as having had a myocardial infarction or similar cardiac event.

In specific embodiments of the methods of treatment provided herein, the stimulated PDACs, e.g., IL-1 β-stimulated PDACs are administered with stem cells (that is, stem cells that are not PDACs), myoblasts, myocytes, cardiomyoblasts, cardiomyocytes, or progenitors of myoblasts, myocytes, cardiomyoblasts, and/or cardiomyocytes.

In a specific embodiment, the methods of treatment provided herein comprise administering stimulated PDACs, e.g., IL-1 β-stimulated PDACs, e.g., a therapeutic composition comprising the stimulated cells, to a patient with a disease of the heart or circulatory system; and evaluating the patient for improvements in cardiac function, wherein the therapeutic cell composition is administered as a matrix-cell complex. In certain embodiments, the matrix is a scaffold, preferably bioabsorbable, comprising at least the cells.

Stimulated PDACs, e.g., IL-1 β-stimulated PDACs, and populations of such cells, can be provided therapeutically or prophylactically to an individual, e.g., an individual having a disease, disorder or condition of, or affecting, the heart or circulatory system. Such diseases, disorders or conditions can include congestive heart failure due to atherosclerosis, cardiomyopathy, or cardiac injury, e.g., an ischemic injury, such as from myocardial infarction or wound (acute or chronic).

In certain embodiments, the individual is administered a therapeutically effective amount of stimulated PDACs, e.g., IL-1 β-stimulated PDACs, e.g., in a population of stimulated cells that comprise the PDACs. In a specific embodiment, the population comprises about 50% stimulated PDACs. In another specific embodiment, the population is a substantially homogeneous population of stimulated PDACs. In other embodiments the population comprises at least about 5%, 10%, 15%, 20%, 25%, 30%, 35%, 40%, 45%, 50%, 55%, 60%, 65%, 70%, 75%, 80%, 85% 90%, 95%, 98% or 99% stimulated PDACs.

The stimulated PDACs, e.g., IL-1 β-stimulated PDACs may be administered to an individual in the form of a therapeutic composition comprising the cells and another therapeutic agent, such as insulin-like growth factor (IGF), platelet-derived growth factor (PDGF), epidermal growth factor (EGF), fibroblast growth factor (FGF), vascular endothelial growth factor (VEGF), hepatocyte growth factor (HGF), interleukin 18 (IL-8), an antithrombogenic agent (e.g., heparin, heparin derivatives, urokinase, or PPack (dextrophenylalanine proline arginine chloromethylketone), an antithrombin compound, a platelet receptor antagonist, an anti-thrombin antibody, an anti-platelet receptor antibody, aspirin, dipyridamole, protamine, hirudin, a prostaglandin inhibitor, and/or a platelet inhibitor), an antiapoptotic agent (e.g., erythropoietin (Epo), an Epo derivative or analog, or their salts, thrombopoietin (Tpo), IGF-I, IGF-II, hepatocyte growth factor (HGF), or a caspase inhibitor), an anti-inflammatory agent (e.g., a p38 MAP kinase inhibitor, a statin, in IL-6 inhibitor, an IL-1 inhibitor, Pemirolast, Tranilast, Remicade, Sirolimus, and/or a nonsteroidal anti-inflammatory compound (e.g., acetylsalicylic acid, ibuprofen, Tepoxalin, Tolmetin, or Suprofen)), an immunosuppressive or immunomodulatory agent (e.g., a calcineurin inhibitor, for example cyclosporine, Tacrolimus, an mTOR inhibitor such as Sirolimus or Everolimus; an anti-proliferative such as azathioprine and/or mycophenolate mofetil; a corticosteroid, e.g., prednisolone or hydrocortisone; an antibody such as a monoclonal anti-IL-2Ra receptor antibody, Basiliximab, Daclizuma, polyclonal anti-T-cell antibodies such as anti-thymocyte globulin (ATG), anti-lymphocyte globulin (ALG), and/or the monoclonal anti-T cell antibody OKT3, or adherent placental stem cells as described in U.S. Pat. No. 7,468,276, and U.S. Patent Application Publication No. 2007/0275362, the disclosures of each of which are incorporated herein by reference in their entireties), and/or an antioxidant (e.g., probucol; vitamins A, C, and/or E, coenzyme Q-10, glutathione, L cysteine, N-acetylcysteine, or an antioxidant derivative, analog or salt of any of the foregoing). In certain embodiments, therapeutic compositions comprising the PDACs further comprise one or more additional cell types, e.g., adult cells (for example, fibroblasts or endodermal cells), stem cells and/or progenitor cells. Such therapeutic agents and/or one or more additional types of cells can be administered to an individual in need thereof individually or in combinations or two or more such compounds or agents.

In certain embodiments, the individual to be treated is a mammal. In a specific embodiment the individual to be treated is a human. In specific embodiments, the individual is a livestock animal or a domestic animal. In other specific embodiments, the individual to be treated is a horse, sheep, cow or steer, pig, dog or cat.

### 5.1.2 Treatment of Ischemic Disease

In certain embodiments, provided herein is a method of treating an individual having a disruption of blood flow, e.g., in the peripheral vasculature, comprising administering to the individual a therapeutically-effective amount of stimulated PDACs, e.g., IL-1 β-stimulated PDACs. In certain specific embodiments, the ischemia is peripheral arterial disease (PAD), e.g., is critical limb ischemia (CLI). In certain other embodiments, the ischemia is peripheral vascular disease (PVD), peripheral arterial disease, ischemic vascular disease, ischemic heart disease, or ischemic renal disease.

In a specific embodiment, said disruption of flow of blood is critical limb ischemia. In another more specific embodiment, said CLI is a severe blockage in the arteries of the lower extremities, which markedly reduces blood-flow. In another more specific embodiment said CLI is characterized by ischemic rest pain, severe pain in the legs and feet while the individual is not moving, non-healing sores on the feet or legs, pain or numbness in the feet, shiny, smooth, dry skin of the legs or feet, thickening of the toenails, absent or diminished pulse in the legs or feet, open sores, skin infections or ulcers that do not heal, and/or dry gangrene (dry, black skin) of the legs or feet. In another specific embodiment, the individual having CLI has experienced loss of at least one digit and/or whole limb. In another specific embodiment of the method, said therapeutically effective amount is a number of stimulated PDACs, e.g., IL-1 β-stimulated PDACs that results in elimination of, a detectable improvement in, lessening of the severity of, or slowing of the progression of one or more symptoms of, loss of limb function and/or oxygen deprivation (hypoxia/anoxia) attributable to a disruption of the flow of blood in the peripheral vasculature of the individual. In another specific embodiment, said therapeutically effective amount of isolated stimulated PDACs, e.g., IL-1 β-stimulated PDACs is administered to said individual prophylactically, e.g., to reduce or eliminate tissue damage caused by a second or subsequent disruption of flow of blood in or around the limb following said disruption of flow of blood.

In other embodiments, the stimulated PDACs, e.g., IL-1 β-stimulated PDACs may be used in the treatment of stroke, e.g., ischemic stroke, e.g., treatment of stroke by promotion of angiogenesis in an ischemic area of the CNS. In one aspect, provided herein is a method of treating an individual who has a disruption of the flow of blood in or around the individual's brain, e.g., who has a symptom or neurological deficit attributable to a disruption of the flow of blood in or around the individual's brain or central nervous system (CNS), comprising administering to said individual a therapeutically effective amount of isolated tissue culture plastic-adherent human placental cells, wherein said isolated placental cells have characteristics of multipotent cells or stem cells. In certain embodiments, the disruption of flow of blood results in anoxic injury or hypoxic injury to the individual's brain or CNS. As contemplated herein, treatment of a symptom or neurological deficit in an individual attributable to a disruption of the flow of blood in or around the individual's brain includes treatment of symptoms or neurological deficits attributable to reperfusion injury that may accompany such a disruption of flow of blood in or around the individual's brain.

In addition to being angiogenic, the stimulated placental cells (e.g., IL-1 β-stimulated PDACs, as described below) provided herein are neuroprotective. In certain embodiments, the stimulated placental cells are neuroprotective in a low-oxygen environment, e.g., under hypoxic conditions (e.g., less than about 5% O₂). In certain embodiments, the stimulated placental stem cells, when contacted with neurons or other neural cells, or astrocytes, increase the health of the neurons, neural cells, or astrocytes, e.g., as seen by an increase in neurite length in vitro in a co-culture of PDACs and neurons. In certain other embodiments, PDACs, e.g., IL-1 β-stimulated PDACs reduce the concentration of one or more reactive oxygen species in a hypoxic environment. Further, in certain embodiments, the stimulated placental cells (e.g., PDACs) secrete one or more of the neurotrophic cytokines BDNF, VEGF, HGF, G-CSF, nerve growth factor (NGF), and/or neurotrophin-3 (NTF3). As such, PDACs, e.g., IL-1 β-stimulated PDACs are useful in the treatment of ischemic injury, both to the CNS and to the PNS, e.g., ischemic injury to the nervous system in the CNS resulting from stroke, or ischemic injury to the nervous system in the PNS resulting from critical limb ischemia or peripheral vascular disease. In other embodiments, the PDACs, e.g., IL-1 β-stimulated PDACs can be used to treat, e.g., multiple sclerosis, amyotrophic lateral sclerosis, Parkinson's disease, Alzheimer's disease, and/or peripheral neuropathies, e.g., diabetic neuropathy.

### 5.1.3 Treatment of Diabetic Foot Ulcer

Provided herein are methods of treating diabetic foot ulcer (DFU) in a subject in need thereof, comprising administering to the subject a therapeutically effective amount of stimulated PDACs, e.g., IL-1β-stimulated PDACs. In a specific embodiment, said stimulated PDACs are formulated as a pharmaceutical composition.

In a specific embodiment, a subject with DFU treated in accordance with the methods provided herein has type I diabetes. In another specific embodiment, a subject with DFU treated in accordance with the methods provided herein has type II diabetes. In certain embodiments, a subject treated in accordance with the methods provided herein has more than one DFU, i.e., the subject has more than one DFU on a single foot, or at least one DFU on each foot. In a specific embodiment, the subject has one or more DFU at the bottom of one foot, or both feet.

In certain embodiments, a subject with DFU treated in accordance with the methods provided herein has peripheral neuropathy, e.g., damage to one or more of the nerves in the legs and/or feet.

In certain embodiments, a subject with DFU treated in accordance with the methods provided herein has DFU with a condition that causes a disruption in the flow of blood in the subject's peripheral vasculature. In a specific embodiment, the subject has peripheral arterial disease (PAD). In certain embodiments, said DFU is caused by and/or associated with PAD.

In certain embodiments, the methods for treating DFU provided herein result in a detectable improvement of one or more symptoms of DFU in a subject treated in accordance with the methods provided herein. Exemplary symptoms of DFU include, without limitation, sores, ulcers, or blisters on the foot and/or lower leg; pain in the foot (or feet) and/or lower leg; difficulty walking; discoloration in the foot (or feet), e.g., the foot (or feet) appear black, blue, and/or red; and signs of infection (e.g., fever, skin redness, and/or swelling).

In certain embodiments, the methods for treating DFU provided herein comprise administering stimulated PDACs (e.g., IL-1-stimulated PDACs or a pharmaceutical composition comprising IL-1β-stimulated PDACs) to a subject having DFU in an amount and for a time sufficient for detectable improvement in one or more indicia of improvement, wherein said indicia of improvement include (i) reduction in ulcer size; (ii) ulcer closure: skin closure of one or more ulcers without drainage or the need for dressing; (iii) retention of ulcer closure for a specified time period following closure, e.g., 2 weeks, 3 weeks, 4 weeks, 5 weeks, or 6 weeks following closure; (iv) time to ulcer closure; (v) improvement in ankle brachial index (ABI), a test that measures blood pressure at the ankle and in the arm while a subject is at rest and then repeated while a subject is in motion (e.g., walking on a treadmill), and which can be used to predict/assess the severity of PAD; (vi) improvement in toe brachial index (TBI), a test analogous to ABI that uses toe blood pressure as opposed to ankle blood pressure; (vii) improvement in transcutaneous oxygen, i.e., the oxygen level in the tissue beneath the skin close to the ulcer (see, e.g., Ruangsetakit et al., J Wound Care, 2010, 19(5):202-6); (viii) improvement in pulse volume recording, which is a noninvasive vascular test in which blood pressure cuffs and a hand-held ultrasound device are used to obtain information about arterial blood flow in the arms and legs; (ix) time to major amputation, e.g., amputation above the ankle; (x) improvement on the Wagner Grading Scale, which assesses ulcer depth and the presence of osteomyelitis or gangrene using a grading system: grade 0 (pre- or post-ulcerative lesion), grade 1 (partial/full thickness ulcer), grade 2 (probing to tendon or capsule), grade 3 (deep with osteitis), grade 4 (partial foot gangrene), and grade 5 (whole foot gangrene); (xi) improvement in Rutherford criteria, which is used for staging of peripheral arterial disease has seven classification stages: Stage 0—Asymptomatic, Stage 1—mild claudication, Stage 2—moderate claudication, Stage 3—severe claudication, Stage 4—rest pain, Stage 5—ischemic ulceration not exceeding ulcer of the digits of the foot, and Stage 6—severe ischemic ulcers or frank gangrene; and (xii) improvement in leg rest pain score, a β-10 scale of pain with 0 being pain free and 10 representing maximum pain.

In certain embodiments, the methods for treating DFU provided herein comprise administering stimulated PDACs (e.g., IL-1β-stimulated PDACs or a pharmaceutical composition comprising IL-1β-stimulated PDACs) to a subject having DFU in an amount and for a time sufficient for detectable improvement in quality of life of the subject as assessed by, e.g., (i) a 36-item Short Form Health Survey (SF-36) (see, e.g., Ware et al., Medical Care 30(6):473-483); (ii) the Diabetic Foot Ulcer Scale Short Form (DFS-SF), which measures the impact of diabetic foot ulcer on quality of life (see, e.g., Bann et al., Pharmacoeconomics, 2003, 21(17):1277-90); (iii) the Patient Global Impression of Change Scale, to assess changes in neuropathy over time (see, e.g., Kamper et al., J. Man. Manip. Ther., 2009, 17(3):163-170); and/or (iv) the EuroQol5D (EQ-5D™) Scale, which is a health questionnaire used to obtain a descriptive profile and single index value for health status of a patient.

In a specific embodiment of the methods of treatment of DFU described herein, the stimulated PDACs (e.g., IL-1β-stimulated PDACs or a pharmaceutical composition comprising IL-1β-stimulated PDACs) are administered by injection. In another specific embodiment of the methods of treatment of DFU described herein, the stimulated PDACs (e.g., IL-1β-stimulated PDACs or a pharmaceutical composition comprising IL-1β-stimulated PDACs) are administered to a subject being treated by implantation in said subject of a matrix or scaffold comprising placental cells.

In a specific embodiment of the methods of treatment of DFU described herein, the stimulated PDACs (e.g., IL-1β-stimulated PDACs or a pharmaceutical composition comprising IL-1β-stimulated PDACs) are administered intramuscularly. In another specific embodiment of the methods of treatment of DFU described herein, the stimulated PDACs (e.g., IL-1β-stimulated PDACs or a pharmaceutical composition comprising IL-1β-stimulated PDACs) are administered intravenously. In another specific embodiment of the methods of treatment of DFU described herein, stimulated PDACs (e.g., IL-1β-stimulated PDACs or a pharmaceutical composition comprising IL-1β-stimulated PDACs) are administered subcutaneously. In another specific embodiment of the methods of treatment of DFU described herein, the stimulated PDACs (e.g., IL-1β-stimulated PDACs or a pharmaceutical composition comprising IL-1β-stimulated PDACs) are administered locally. In another specific embodiment of the methods of treatment of DFU described herein, the stimulated PDACs (e.g., IL-IP-stimulated PDACs or a pharmaceutical composition comprising IL-1β-stimulated PDACs) are administered systemically. In certain embodiments, the methods of treatment of DFU described herein comprise administration of about 1×10⁶, 3×10⁶, 5×10⁶, 1×10⁷, 3×10⁷, 5×10⁷, 1×10⁸, 3×10⁸, 5×10⁸, 1×10⁹, 5×10⁹, or 1×10¹⁰ stimulated PDACs (e.g., IL-IP-stimulated PDACs or a pharmaceutical composition comprising IL-1β-stimulated PDACs). In certain embodiments, the methods of treatment of DFU described herein comprise administration of about 1×10⁶ to 3×10⁶, 3×10⁶ to 5×10⁶, 5×10⁶ to 1×10⁷, 1×10⁷ to 3×10⁷, 3×10⁷ to 5×10⁷, 5×10⁷ to 1×10⁸, 1×10⁸ to 3×10⁸, 3×10⁸ to 5×10⁸, 5×10⁸ to 1×10⁹, 1×10⁹ to 5×10⁹, or 5×10⁹ to 1×10¹⁰ stimulated PDACs (e.g., IL-1β-stimulated PDACs or a pharmaceutical composition comprising IL-1β-stimulated PDACs). In a specific embodiment, the methods of treatment of DFU described herein comprise administration of about 3×10⁶ stimulated PDACs (e.g., IL-1β-stimulated PDACs). In another specific embodiment, the methods of treatment of DFU described herein comprise administration of about 1×10⁷ stimulated PDACs (e.g., IL-1β-stimulated PDACs). In another specific embodiment, the methods of treatment of DFU described herein comprise administration of about 3×10⁷ stimulated PDACs (e.g., IL-1β-stimulated PDACs).

In a specific embodiment of the methods of treatment of DFU described herein, the stimulated PDACs (e.g., IL-1β-stimulated PDACs or a pharmaceutical composition comprising IL-1β-stimulated PDACs) are administered intramuscularly with one week between administrations, e.g., stimulated PDACs are administered on day 1 (the first day of administration) and a second dose of stimulated PDACs (e.g., IL-1β-stimulated PDACs or a pharmaceutical composition comprising IL-1β-stimulated PDACs) is administered one week later (i.e., on day 8). In another specific embodiment, the methods comprise administration of about 3×10⁶ stimulated PDACs (e.g., IL-1β-stimulated PDACs) on each day of administration (i.e., on days 1 and 8). In another specific embodiment, the methods comprise administration of about 1×10⁷ stimulated PDACs (e.g., IL-1β-stimulated PDACs) on each day of administration (i.e., on days 1 and 8). In another specific embodiment, the methods comprise administration of about 3×10⁷ stimulated PDACs (e.g., IL-1β-stimulated PDACs) on each day of administration (i.e., on days 1 and 8). In another specific embodiment, the subject to whom the stimulated PDACs (e.g., IL-1β-stimulated PDACs) are administered has PAD.

### 5.2 Isolated Placental Cells and Isolated Placental Cell Populations

The placental cells that serve as the basis for generation of stimulated PDACs, for example IL-1 β-stimulated PDACs, are cells obtainable from a placenta or part thereof, that adhere to a tissue culture substrate and have characteristics of multipotent cells or stem cells, but are not trophoblasts. This Section (5.2) describes the placental cells that represent the source of cells that can be used to generate stimulated PDACs, for example IL-1 β-stimulated PDACs.

The placental cells that serve as the basis for generation of stimulated PDACs can be either fetal or maternal in origin (that is, can have the genotype of either the fetus or mother, respectively). Preferably, the placental cells and populations thereof are fetal in origin. As used herein, the phrase “fetal in origin” or “non-maternal in origin” indicates that the isolated placental cells or populations of isolated placental cells are obtained from the umbilical cord or placental structures associated with the fetus, i.e., that have the fetal genotype. As used herein, the phrase “maternal in origin” indicates that the cells or populations of cells are obtained from a placental structures associated with the mother, e.g., which have the maternal genotype. Isolated placental cells or populations of cells comprising the isolated placental cells, can comprise isolated placental cells that are solely fetal or maternal in origin, or can comprise a mixed population of isolated placental cells of both fetal and maternal origin. The isolated placental cells, and populations of cells comprising the stimulated isolated placental cells, can be identified and selected by the morphological, marker, and culture characteristics discussed below. In certain embodiments, any of the placental cells, e.g., placental stem cells or placental multipotent cells described herein, are autologous to a recipient, e.g., an individual who has a disease or disorder of the circulatory system. In certain other embodiments, any of the placental cells, e.g., placental stem cells or placental multipotent cells described herein, are heterologous to a recipient, e.g., an individual who has a disease or disorder of the circulatory system.

In certain embodiments, the placental stem cells described herein that serve as the basis for generation of stimulated PDACs (e.g., the PDACs described in Sections 5.2.1, 5.2.2, and 5.2.3, Infra) are stimulated with one or more pro-inflammatory cytokines. In certain embodiments, the placental stem cells described herein that serve as the basis for generation of stimulated PDACs (e.g., the PDACs described in Sections 5.2.1, 5.2.2, and 5.2.3, Infra) have been stimulated with one or more of IL-1 α, IL-1 β, IL-6, IL-8, IL-18, TNF-α, or INF-γ. In a specific embodiment, the placental stem cells described herein that serve as the basis for generation of stimulated PDACs (e.g., the PDACs described in Sections 5.2.1, 5.2.2, and 5.2.3, Infra) have been stimulated with IL-1 β.

### 5.2.1 Physical and Morphological Characteristics

The isolated placental cells used to generate the stimulated PDACs described herein, when cultured in primary cultures or in cell culture, adhere to the tissue culture substrate, e.g., tissue culture container surface (e.g., tissue culture plastic), or to a tissue culture surface coated with extracellular matrix or ligands such as laminin, collagen (e.g., native or denatured), gelatin, fibronectin, ornithine, vitronectin, and extracellular membrane protein (e.g., MATRIGEL® (BD Discovery Labware, Bedford, Mass.)). The isolated placental cells used to generate the stimulated PDACs described herein assume a generally fibroblastoid, stellate appearance in culture, with a number of cytoplasmic processes extending from the central cell body. The cells are, however, morphologically distinguishable from fibroblasts cultured under the same conditions, as the isolated placental cells exhibit a greater number of such processes than do fibroblasts. Morphologically, the isolated placental cells used to generate the stimulated PDACs described herein are also distinguishable from hematopoietic stem cells, which generally assume a more rounded, or cobblestone, morphology in culture.

In certain embodiments, the isolated placental cells used to generate the stimulated PDACs described herein, when cultured in a growth medium, develop embryoid-like bodies. Embryoid-like bodies are noncontiguous clumps of cells that can grow on top of an adherent layer of proliferating isolated placental cells. The term “embryoid-like” is used because the clumps of cells resemble embryoid bodies, clumps of cells that grow from cultures of embryonic stem cells. Growth medium in which embryoid-like bodies can develop in a proliferating culture of isolated placental cells includes medium comprising, e.g., DMEM-LG (e.g., from Gibco); 2% fetal calf serum (e.g., from Hyclone Labs.); lx insulin-transferrin-selenium (ITS); lx linoleic acid-bovine serum albumin (LA-BSA); 10⁻⁹ M dexamethasone (e.g., from Sigma); 10⁻⁴ M ascorbic acid 2-phosphate (e.g., from Sigma); epidermal growth factor 10 ng/mL (e.g., from R&D Systems); and platelet-derived growth factor (PDGF-BB) 10 ng/mL (e.g., from R&D Systems).

### 5.2.2 Cell Surface, Molecular and Genetic Markers

The isolated placental cells used to generate the stimulated PDACs described herein are tissue culture plastic-adherent human placental cells that have characteristics of multipotent cells or stem cells, and express a plurality of markers that can be used to identify and/or isolate the cells, or populations of cells that comprise the stem cells. In certain embodiments, the placental cells used to generate the stimulated PDACs described herein are angiogenic, e.g., in vitro or in vivo. The isolated placental cells, and placental cell populations used to generate the stimulated PDACs described herein include placental cells and placental cell-containing cell populations obtained directly from the placenta, or any part thereof (e.g., chorion, placental cotyledons, or the like). The placental cell populations used to generate the stimulated PDACs described herein also include populations of (that is, two or more) isolated placental cells in culture, and a population in a container, e.g., a bag. The placental cells used to generate the stimulated PDACs described herein are not bone marrow-derived mesenchymal cells, adipose-derived mesenchymal stem cells, or mesenchymal cells obtained from umbilical cord blood, placental blood, or peripheral blood. The placental cells, e.g., placental multipotent cells and placental cells, used to generate the stimulated PDACs described herein, which are useful in the methods and compositions described herein are described herein and, e.g., in U.S. Pat. Nos. 7,311,904; 7,311,905; and 7,468,276; and in U.S. Patent Application Publication No. 2007/0275362, the disclosures of which are hereby incorporated by reference in their entireties.

In certain other embodiments, the placental cells used to generate the stimulated PDACs described herein are isolated placental multipotent cells. In one embodiment, said cells, e.g., the cells used to generate the stimulated PDACs described herein, are CD34⁻, CD10⁺ and CD105⁺ as detected by flow cytometry. In another specific embodiment, the isolated CD34⁻, CD10⁺, CD105⁺ placental cells used to generate the stimulated PDACs described herein have the potential to differentiate into cells of a neural phenotype, cells of an osteogenic phenotype, and/or cells of a chondrogenic phenotype. In another specific embodiment, the isolated CD34⁻, CD10⁺, CD105⁺ placental cells used to generate the stimulated PDACs described herein are additionally CD200⁺. In another specific embodiment, the isolated CD34⁻, CD10⁺, CD105 placental cells used to generate the stimulated PDACs described herein are additionally CD45⁻ or CD90⁺. In another specific embodiment, the isolated CD34⁻, CD10⁺, CD105⁺ placental cells used to generate the stimulated PDACs described herein are additionally CD45− and CD90⁺, as detected by flow cytometry. In another specific embodiment, the isolated CD34⁻, CD10⁺, CD105⁺, CD200⁺ placental cells used to generate the stimulated PDACs described herein are additionally CD90⁺ or CD45⁻, as detected by flow cytometry. In another specific embodiment, the isolated CD34⁻, CD10⁺, CD105⁺, CD200⁺ placental cells used to generate the stimulated PDACs described herein are additionally CD90⁺ and CD45⁻, as detected by flow cytometry, i.e., the cells are CD34⁻, CD10⁺, CD45⁻, CD90⁺, CD105⁺ and CD200⁺. In another specific embodiment, said CD34⁻, CD10⁺, CD45⁻, CD90⁺, CD105⁺, CD200⁺ cells are additionally CD80- and CD86⁻. In another specific embodiment, the isolated placental cells used to generate the stimulated PDACs described herein have been stimulated by one or more cytokines. In a specific embodiment, said cytokine is a pro-inflammatory cytokine. In another specific embodiment, said cytokine is IL-1β.

In certain embodiments, the isolated placental cells used to generate the stimulated PDACs described herein are CD34⁻, CD10⁺, CD105⁺ and CD200⁺, and one or more of CD38⁻, CD45⁻, CD80⁻, CD86⁻, CD133⁻, HLA-DR,DP,DQ⁻, SSEA3⁻, SSEA4⁻, CD29⁺, CD44⁺, CD73⁺, CD90⁺, CD105⁺, HLA-A,B,C⁺, PDL1⁺, ABC-p⁺, and/or OCT-4⁺, as detected by flow cytometry. In other embodiments, any of the CD34⁻, CD10⁺, CD105⁺ placental cells used to generate the stimulated PDACs described herein are additionally one or more of CD29⁺, CD38⁻, CD44⁺, CD54⁺, SH3⁺ or SH4⁺. In another specific embodiment, the cells are additionally CD44⁺. In another specific embodiment of any of the isolated CD34⁻, CD10⁺, CD105⁺ placental cells above, the cells are additionally one or more of CD11T, CD133⁻, KDR⁻ (VEGFR2⁻), HLA-A,B,C⁺, HLA-DP,DQ,DR⁻, or Programmed Death-1 Ligand (PDL1)⁺, or any combination thereof. In another specific embodiment, the isolated placental stem cells used to generate the stimulated PDACs described herein have been stimulated by one or more cytokines. In a specific embodiment, said cytokine is a pro-inflammatory cytokine. In another specific embodiment, said cytokine is IL-1β.

In another embodiment, the CD34⁻, CD10⁺, CD105⁺ placental cells used to generate the stimulated PDACs described herein are additionally one or more of CD13⁺, CD29⁺, CD33⁺, CD38⁻, CD44⁺, CD45⁻, CD54⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{low}, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, or Programmed Death-1 Ligand (PDL1)⁺, or any combination thereof. Ina other embodiment, the CD34⁻, CD10⁺, CD105⁺ placental cells used to generate the stimulated PDACs described herein are additionally CD13⁺, CD29⁺, CD33⁺, CD38⁻, CD44⁺, CD45⁻, CD54/ICAM⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁺, CD144/VE-cadherin^{low}, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, and Programmed Death-1 Ligand (PDL1)⁺.

In another specific embodiment, any of the placental cells used to generate the stimulated PDACs described herein are additionally ABC-p⁺, as detected by flow cytometry, or OCT-4+(POU5F1⁺), as determined by RT-PCR, wherein ABC-p⁺ is a placenta-specific ABC transporter protein (also known as breast cancer resistance protein (BCRP) and as mitoxantrone resistance protein (MXR)), and OCT-4 is the Octamer-4 protein (POU5F1). In another specific embodiment, any of the placental cells used to generate the stimulated PDACs described herein are additionally SSEA3⁻ or SSEA4⁻, as determined by flow cytometry, wherein SSEA3 is Stage Specific Embryonic Antigen 3, and SSEA4 is Stage Specific Embryonic Antigen 4. In another specific embodiment, any of the placental cells used to generate the stimulated PDACs described herein are additionally SSEA3⁻ and SSEA4⁻.

In another specific embodiment, any of the isolated placental cells used to generate the stimulated PDACs described herein are additionally one or more of MHC-I⁺ (e.g., HLA-A,B,C⁺), MHC-II⁻ (e.g., HLA-DP,DQ,DR⁻) or HLA-G⁻. In another specific embodiment, any of the isolated placental stem cells used to generate the stimulated PDACs described herein described herein are additionally one or more of MHC-I⁺ (e.g., HLA-A,B,C⁺), MHC-II⁻ (e.g., HLA-DP,DQ,DR⁻) and HLA-G⁻.

Also provided herein are populations of the isolated placental cells used to generate the stimulated PDACs described herein, or populations of cells, e.g., populations of placental cells, comprising, e.g., placental stem cells that are enriched for the isolated placental cells that are useful in the methods and compositions disclosed herein. Preferred populations of cells comprising the isolated placental cells used to generate the stimulated PDACs described herein, wherein the populations of cells comprise, e.g., at least 10%, 15%, 20%, 25%, 30%, 35%, 40%, 45%, 50%, 55%, 60%, 65%, 70%, 75%, 80%, 85%, 90%, 95% or 98% isolated CD10⁺, CD105⁺ and CD34− placental cells; that is, at least 10%, 15%, 20%, 25%, 30%, 35%, 40%, 45%, 50%, 55%, 60%, 65%, 70%, 75%, 80%, 85%, 90%, 95% or 98% of cells in said population are isolated CD10⁺, CD105⁺ and CD34⁻ placental cells. In a specific embodiment, the isolated CD34⁻, CD10⁺, CD105⁺ placental cells are additionally CD200⁺. In another specific embodiment, the isolated CD34⁻, CD10⁺, CD105⁺, CD200⁺ placental cells are additionally CD90⁺ or CD45⁻, as detected by flow cytometry. In another specific embodiment, the isolated CD34⁻, CD10⁺, CD105⁺, CD200⁺ placental cells are additionally CD90⁺ and CD45⁻, as detected by flow cytometry. In another specific embodiment, any of the isolated CD34⁻, CD10⁺, CD105⁺ placental cells described above are additionally one or more of CD29⁺, CD38⁻, CD44⁺, CD54⁺, SH3⁺ or SH4⁺. In another specific embodiment, the isolated CD34⁻, CD10⁺, CD105⁺ the isolated placental cells used to generate the stimulated PDACs described herein, or isolated CD34⁻, CD10⁺, CD105⁺, CD200⁺ the isolated placental cells used to generate the stimulated PDACs described herein, are additionally CD44⁺. In a specific embodiment of any of the populations of cells comprising isolated CD34⁻, CD10⁺, CD105⁺ placental cells above, the isolated placental cells are additionally one or more of CD13⁺, CD29⁺, CD33⁺, CD38⁻, CD44⁺, CD45⁻, CD54⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁻), CD106/VCAM⁺, CD117⁻, CD144/VE-cadherin^{low}, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, or Programmed Death-1 Ligand (PDL1)⁺, or any combination thereof. In another specific embodiment, the CD34⁻, CD10⁺, CD105⁺ cells used to generate the stimulated PDACs described herein are additionally CD13⁺, CD29⁺, CD33⁺, CD38⁻, CD44⁺, CD45⁻, CD54/ICAM⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁺, CD144/VE-cadherin^{low}, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, and Programmed Death-1 Ligand (PDL1)⁺.

In certain embodiments, the isolated placental cells used to generate the stimulated PDACs useful in the methods and compositions described herein are one or more, or all, of CD10⁺, CD29⁺, CD34⁻, CD38⁻, CD44⁺, CD45⁻, CD54⁺, CD90⁺, SH2⁺, SH3, SH4⁺, SSEA3⁻, SSEA4⁻, OCT-4⁺, and ABC-p, wherein said isolated placental cells are obtained by physical and/or enzymatic disruption of placental tissue. In a specific embodiment, the isolated placental cells are OCT-4⁺ and ABC-p⁺. In another specific embodiment, the isolated placental cells used to generate the stimulated PDACs described herein are OCT-4⁺ and CD34⁻, wherein said isolated placental cells have at least one of the following characteristics: CD10⁺, CD29⁺, CD44⁺, CD45⁻, CD54⁺, CD90⁺, SH3, SH4⁺, SSEA3⁻, and SSEA4⁻. In another specific embodiment, the isolated placental cells used to generate the stimulated PDACs described herein are OCT-4⁺, CD34⁻, CD10⁺, CD29⁺, CD44⁺, CD45⁻, CD54⁺, CD90⁺, SH3⁺, SH4⁺, SSEA3⁻, and SSEA4⁻. In another embodiment, the isolated placental cells used to generate the stimulated PDACs described herein are OCT-4⁺, CD34⁻, SSEA3⁻, and SSEA4⁻. In another specific embodiment, the isolated placental cells used to generate the stimulated PDACs described herein are OCT-4⁺ and CD34⁻, and is either SH2⁺ or SH3⁺. In another specific embodiment, the isolated placental cells used to generate the stimulated PDACs described herein are OCT-4⁺, CD34⁻, SH2⁺, and SH3⁺. In another specific embodiment, the isolated placental cells used to generate the stimulated PDACs described herein are OCT-4⁺, CD34⁻, SSEA3⁻, and SSEA4⁻, and are either SH2⁺ or SH3⁺. In another specific embodiment, the isolated placental cells used to generate the stimulated PDACs described herein are OCT-4⁺ and CD34⁻, and either SH2⁺ or SH3⁺, and is at least one of CD10⁺, CD29⁺, CD44⁺, CD45⁻, CD54⁺, CD90⁺, SSEA3⁻, or SSEA4⁻. In another specific embodiment, the isolated placental cells used to generate the stimulated PDACs described herein are OCT-4⁺, CD34⁻, CD10⁺, CD29⁺, CD44⁺, CD45⁻, CD54⁺, CD90⁺, SSEA3⁻, and SSEA4⁻, and either SH2⁺ or SH3⁺.

In another embodiment, the isolated placental stem used to generate the stimulated PDACs described herein, which are useful in the methods and compositions disclosed herein are SH2⁺, SH3⁺, SH4⁺ and OCT-4⁺. In another specific embodiment, the isolated placental cells are CD10⁺, CD29⁺, CD44⁺, CD54⁺, CD90⁺, CD34⁻, CD45⁻, SSEA3⁻, or SSEA4⁻. In another embodiment, the isolated placental cells used to generate the stimulated PDACs described herein are SH2⁺, SH3⁺, SH4⁺, SSEA3⁻ and SSEA4⁻. In another specific embodiment, the isolated placental cells are SH2⁺, SH3⁺, SH4⁺, SSEA3- and SSEA4⁻, CD10⁺, CD29⁺, CD44⁺, CD54⁺, CD90⁺, OCT-4⁺, CD34⁻ or CD45⁻.

In another embodiment, the isolated placental cells used to generate the stimulated PDACs described herein, which are useful in the methods and compositions disclosed herein are CD10⁺, CD29⁺, CD34⁻, CD44⁺, CD45⁻, CD54⁺, CD90⁺, SH2⁺, SH3⁺, and SH4⁺; wherein said isolated placental cells are additionally one or more of OCT-4⁺, SSEA3⁻ or SSEA4⁻.

In certain embodiments, isolated placental cells used to generate the stimulated PDACs described herein, which are useful in the methods and compositions disclosed herein are CD200⁺ or HLA-G⁻. In a specific embodiment, the isolated placental cells used to generate the stimulated PDACs described herein are CD200⁺ and HLA-G⁻. In another specific embodiment, the isolated placental cells used to generate the stimulated PDACs described herein are additionally CD73⁺ and CD105⁺. In another specific embodiment, the isolated placental cells used to generate the stimulated PDACs described herein are additionally CD34⁻, CD38⁻ or CD45⁻. In another specific embodiment, the isolated placental cells used to generate the stimulated PDACs described herein are additionally CD34⁻, CD38⁻ and CD45⁻. In another specific embodiment, said stem cells are CD34⁻, CD38⁻, CD45⁻, CD73⁺ and CD105⁺. In another specific embodiment, said isolated CD200⁺ or HLA-G⁻ placental cells facilitate the formation of embryoid-like bodies in a population of placental cells comprising the isolated placental cells, under conditions that allow the formation of embryoid-like bodies. In another specific embodiment, the isolated placental cells used to generate the stimulated PDACs described herein are isolated away from placental cells that are not stem or multipotent cells. In another specific embodiment, said isolated placental cells are isolated away from placental cells that do not display these markers.

In another embodiment, a cell population useful in the methods and compositions described herein is a population of cells comprising, e.g., that is enriched for, CD200⁺, HLA-G⁻ stem cells. In a specific embodiment, said population is a population of placental cells used to generate the stimulated PDACs described herein. In various embodiments, at least about 10%, at least about 20%, at least about 30%, at least about 40%, at least about 50%, or at least about 60% of cells in said cell population are isolated CD200⁺, HLA-G⁻ placental cells. Preferably, at least about 70% of cells in said cell population are isolated CD200⁺, HLA-G⁻ placental cells. More preferably, at least about 90%, 95%, or 99% of said cells are isolated CD200⁺, HLA-G⁻ placental cells. In a specific embodiment of the cell populations, said isolated CD200⁺, HLA-G⁻ placental cells are also CD73⁺ and CD105⁺. In another specific embodiment, said isolated CD200⁺, HLA-G⁻ placental cells are also CD34⁻, CD38⁻ or CD45⁻. In another specific embodiment, said isolated CD200⁺, HLA-G⁻ placental cells are also CD34⁻, CD38⁻, CD45⁻, CD73⁺ and CD105⁺.

In another embodiment, said cell population produces one or more embryoid-like bodies when cultured under conditions that allow the formation of embryoid-like bodies. In another specific embodiment, said cell population is isolated away from placental cells that are not stem cells. In another specific embodiment, said isolated CD200⁺, HLA-G⁻ placental cells are isolated away from placental cells that do not display these markers.

In another embodiment, the isolated placental cells useful in the methods and compositions described herein are CD73⁺, CD105⁺, and CD200⁺. In another specific embodiment, the isolated placental cells used to generate the stimulated PDACs described herein are HLA-G⁻. In another specific embodiment, the isolated placental cells used to generate the stimulated PDACs described herein are CD34⁻, CD38⁻ or CD45⁻. In another specific embodiment, the isolated placental cells used to generate the stimulated PDACs described herein are CD34⁻, CD38⁻ and CD45⁻. In another specific embodiment, the isolated placental cells used to generate the stimulated PDACs described herein are CD34⁻, CD38⁻, CD45⁻, and HLA-G⁻. In another specific embodiment, the isolated CD73⁺, CD105⁺, and CD200⁺ placental cells used to generate the stimulated PDACs described herein facilitate the formation of one or more embryoid-like bodies in a population of placental cells comprising the isolated placental cells, when the population is cultured under conditions that allow the formation of embryoid-like bodies. In another specific embodiment, the isolated placental cells used to generate the stimulated PDACs described herein are isolated away from placental cells that are not the isolated placental cells. In another specific embodiment, the isolated placental cells used to generate the stimulated PDACs described herein are isolated away from placental cells that do not display these markers.

In another embodiment, a cell population used to generate the stimulated PDACs described herein, which are useful in the methods and compositions described herein is a population of cells comprising, e.g., that is enriched for, isolated CD73⁺, CD105⁺, CD200⁺ placental cells. In various embodiments, at least about 10%, at least about 20%, at least about 30%, at least about 40%, at least about 50%, or at least about 60% of cells in said cell population are isolated CD73⁺, CD105⁺, CD200⁺ placental cells. In another embodiment, at least about 70% of said cells in said population of cells are isolated CD73⁺, CD105⁺, CD200⁺ placental cells. In another embodiment, at least about 90%, 95% or 99% of cells in said population of cells are isolated CD73⁺, CD105⁺, CD200⁺ placental cells. In a specific embodiment of said populations, the isolated placental cells used to generate the stimulated PDACs described herein are HLA-G⁻. In another specific embodiment, the isolated placental cells used to generate the stimulated PDACs described herein are additionally CD34⁻, CD38⁻ or CD45⁻. In another specific embodiment, the isolated placental cells used to generate the stimulated PDACs described herein are additionally CD34⁻, CD38⁻ and CD45⁻. In another specific embodiment, the isolated placental cells used to generate the stimulated PDACs described herein are additionally CD34⁻, CD38⁻, CD45⁻, and HLA-G⁻. In another specific embodiment, said population of cells produces one or more embryoid-like bodies when cultured under conditions that allow the formation of embryoid-like bodies. In another specific embodiment, said population of placental cells is isolated away from placental cells that are not stem cells. In another specific embodiment, said population of placental cells is isolated away from placental cells that do not display these characteristics.

In certain other embodiments, the isolated placental cells used to generate the stimulated PDACs described herein are one or more of CD10⁺, CD29⁺, CD34⁻, CD38⁻, CD44⁺, CD45⁻, CD54⁺, CD90⁺, SH2⁺, SH3, SH4⁺, SSEA3⁻, SSEA4⁻, OCT-4⁺, HLA-G⁻ or ABC-p⁺. In a specific embodiment, the isolated placental cells used to generate the stimulated PDACs described herein are CD10⁺, CD29⁺, CD34⁻, CD38⁻, CD44⁺, CD45⁻, CD54⁺, CD90⁺, SH2⁺, SH3⁺, SH4⁺, SSEA3⁻, SSEA4⁻, and OCT-4⁺. In another specific embodiment, the isolated placental cells used to generate the stimulated PDACs described herein are CD10⁺, CD29⁺, CD34⁻, CD38⁻, CD45⁻, CD54⁺, SH2⁺, SH3⁺, and SH4⁺. In another specific embodiment, the isolated placental cells used to generate the stimulated PDACs described herein are CD10⁺, CD29⁺, CD34⁻, CD38⁻, CD45⁻, CD54⁺, SH2⁺, SH3, SH4⁺ and OCT-4⁺. In another specific embodiment, the isolated placental cells used to generate the stimulated PDACs described herein are CD10⁺, CD29⁺, CD34⁻, CD38⁻, CD44⁺, CD45⁻, CD54⁺, CD90⁺, HLA-G⁻, SH2⁺, SH3⁺, SH4⁺. In another specific embodiment, the isolated placental cells used to generate the stimulated PDACs described herein are OCT-4⁺ and ABC-p⁺. In another specific embodiment, the isolated placental cells used to generate the stimulated PDACs described herein are SH2⁺, SH3Y, SH4⁺ and OCT-4⁺. In another embodiment, the isolated placental cells used to generate the stimulated PDACs described herein are OCT-4⁺, CD34⁻, SSEA3⁻, and SSEA4⁻. In a specific embodiment, said isolated OCT-4⁺, CD34⁻, SSEA3⁻, and SSEA4⁻ placental cells used to generate the stimulated PDACs described herein are additionally CD10⁺, CD29⁺, CD34⁻, CD44⁺, CD45⁻, CD54⁺, CD90⁺, SH2⁺, SH3, and SH4⁺. In another embodiment, the isolated placental cells used to generate the stimulated PDACs described herein are OCT-4⁺ and CD34⁻, and either SH3Y or SH4⁺. In another embodiment, the isolated placental cells used to generate the stimulated PDACs described herein are CD34⁻ and either CD10⁺, CD29⁺, CD44⁺, CD54⁺, CD90⁺, or OCT-4⁺.

In another embodiment, the isolated placental cells used to generate the stimulated PDACs described herein, which are useful in the methods and compositions described herein are CD200⁺ and OCT-4⁺. In a specific embodiment, the isolated placental cells used to generate the stimulated PDACs described herein are CD73⁺ and CD105⁺. In another specific embodiment, said isolated placental cells used to generate the stimulated PDACs described herein are HLA-G⁻. In another specific embodiment, said isolated CD200⁺, OCT-4⁺ placental are CD34⁻, CD38⁻ or CD45⁻. In another specific embodiment, said isolated CD200⁺, OCT-4⁺ placental cells are CD34⁻, CD38⁻ and CD45⁻. In another specific embodiment, said isolated CD200⁺, OCT-4⁺ placental are CD34⁻, CD38⁻, CD45⁻, CD73⁺, CD105⁺ and HLA-G⁻. In another specific embodiment, the isolated CD200⁺, OCT-4⁺ placental cells facilitate the production of one or more embryoid-like bodies by a population of placental cells that comprises the isolated cells, when the population is cultured under conditions that allow the formation of embryoid-like bodies. In another specific embodiment, said isolated CD200⁺, OCT-4⁺ placental cells are isolated away from placental cells that are not stem cells. In another specific embodiment, said isolated CD200⁺, OCT-4⁺ placental cells are isolated away from placental cells that do not display these characteristics.

In another embodiment, a cell population useful in the methods and compositions described herein is a population of cells used to generate the stimulated PDACs described herein, comprising, e.g., that is enriched for, CD200⁺, OCT-4⁺ placental cells. In various embodiments, at least about 10%, at least about 20%, at least about 30%, at least about 40%, at least about 50%, or at least about 60% of cells in said cell population are isolated CD200⁺, OCT-4⁺ placental cells. In another embodiment, at least about 70% of said cells are said isolated CD200⁺, OCT-4⁺ placental cells. In another embodiment, at least about 80%, 90%, 95%, or 99% of cells in said cell population are said isolated CD200⁺, OCT-4⁺ placental cells. In a specific embodiment of the isolated populations, said isolated CD200⁺, OCT-4⁺ placental cells are additionally CD73⁺ and CD105⁺. In another specific embodiment, said isolated CD200⁺, OCT-4⁺ placental cells are additionally HLA-G⁻. In another specific embodiment, said isolated CD200⁺, OCT-4⁺ placental cells are additionally CD34⁻, CD38⁻ and CD45⁻. In another specific embodiment, said isolated CD200⁺, OCT-4⁺ placental cells are additionally CD34⁻, CD38⁻, CD45⁻, CD73⁺, CD105⁺ and HLA-G⁻. In another specific embodiment, the cell population produces one or more embryoid-like bodies when cultured under conditions that allow the formation of embryoid-like bodies. In another specific embodiment, said cell population is isolated away from placental cells that are not isolated CD200⁺, OCT-4⁺ placental cells. In another specific embodiment, said cell population is isolated away from placental cells that do not display these markers.

In another embodiment, the isolated placental cells used to generate the stimulated PDACs described herein, which are useful in the methods and compositions described herein are CD73⁺, CD105⁺ and HLA-G⁻. In another specific embodiment, the isolated CD73⁺, CD105⁺ and HLA-G⁻ placental cells are additionally CD34⁻, CD38⁻ or CD45⁻. In another specific embodiment, the isolated CD73⁺, CD105⁺, HLA-G⁻ placental cells are additionally CD34⁻, CD38⁻ and CD45⁻. In another specific embodiment, the isolated CD73⁺, CD105⁺, HLA-G⁻ placental cells are additionally OCT-4⁺. In another specific embodiment, the isolated CD73⁺, CD105⁺, HLA-G⁻ placental cells are additionally CD200⁺. In another specific embodiment, the isolated CD73⁺, CD105⁺, HLA-G⁻ placental cells are additionally CD34⁻, CD38⁻, CD45⁻, OCT-4⁺ and CD200⁺. In another specific embodiment, the isolated CD73⁺, CD105⁺, HLA-G⁻ placental cells facilitate the formation of embryoid-like bodies in a population of placental cells comprising said cells, when the population is cultured under conditions that allow the formation of embryoid-like bodies. In another specific embodiment, said the isolated CD73⁺, CD105⁺, HLA-G⁻ placental cells are isolated away from placental cells that are not the isolated CD73⁺, CD105⁺, HLA-G⁻ placental cells. In another specific embodiment, said the isolated CD73⁺, CD105⁺, HLA-G⁻ placental cells are isolated away from placental cells that do not display these markers.

In another embodiment, a cell population used to generate the stimulated PDACs described herein, which are useful in the methods and compositions described herein is a population of cells comprising, e.g., a population that is enriched for, isolated CD73⁺, CD105⁺ and HLA-G⁻ placental cells. In various embodiments, at least about 10%, at least about 20%, at least about 30%, at least about 40%, at least about 50%, or at least about 60% of cells in said population of cells are isolated CD73⁺, CD105⁺, HLA-G⁻ placental cells. In another embodiment, at least about 70% of cells in said population of cells are isolated CD73⁺, CD105⁺, HLA-G⁻ placental cells. In another embodiment, at least about 90%, 95% or 99% of cells in said population of cells are isolated CD73⁺, CD105⁺, HLA-G⁻ placental cells. In a specific embodiment of the above populations, said isolated CD73⁺, CD105⁺, HLA-G⁻ placental cells are additionally CD34⁻, CD38⁻ or CD45⁻. In another specific embodiment, said isolated CD73⁺, CD105⁺, HLA-G⁻ placental cells are additionally CD34⁻, CD38⁻ and CD45⁻. In another specific embodiment, said isolated CD73⁺, CD105⁺, HLA-G⁻ placental cells are additionally OCT-4⁺. In another specific embodiment, said isolated CD73⁺, CD105⁺, HLA-G⁻ placental cells are additionally CD200⁺. In another specific embodiment, said isolated CD73⁺, CD105⁺, HLA-G⁻ placental cells are additionally CD34⁻, CD38⁻, CD45⁻, OCT-4⁺ and CD200⁺. In another specific embodiment, said cell population used to generate the stimulated PDACs described herein is isolated away from placental cells that are not CD73⁺, CD105⁺, HLA-G⁻ placental cells. In another specific embodiment, said cell population used to generate the stimulated PDACs described herein is isolated away from placental cells that do not display these markers.

In another embodiment, the isolated placental cells used to generate the stimulated PDACs described herein, which are useful in the methods and compositions described herein are CD73⁺ and CD105⁺ and facilitate the formation of one or more embryoid-like bodies in a population of isolated placental cells comprising said CD73⁺, CD105⁺ cells when said population is cultured under conditions that allow formation of embryoid-like bodies. In another specific embodiment, said isolated CD73⁺, CD105⁺ placental cells are additionally CD34⁻, CD38⁻ or CD45⁻. In another specific embodiment, said isolated CD73⁺, CD105⁺ placental cells are additionally CD34⁻, CD38⁻ and CD45⁻. In another specific embodiment, said isolated CD73⁺, CD105⁺ placental cells are additionally OCT-4⁺. In another specific embodiment, said isolated CD73⁺, CD105⁺ placental cells are additionally OCT-4⁺, CD34⁻, CD38⁻ and CD45⁻. In another specific embodiment, said isolated CD73⁺, CD105⁺ placental cells are isolated away from placental cells that are not said cells. In another specific embodiment, said isolated CD73⁺, CD105⁺ placental cells are isolated away from placental cells that do not display these characteristics.

In another embodiment, a cell population used to generate the stimulated PDACs described herein, which are useful in the methods and compositions described herein is a population of cells comprising, e.g., a population that is enriched for isolated placental cells that are CD73⁺, CD105⁺ and facilitate the formation of one or more embryoid-like bodies in a population of isolated placental cells comprising said cells when said population is cultured under conditions that allow formation of embryoid-like bodies. In various embodiments, at least about 10%, at least about 20%, at least about 30%, at least about 40%, at least about 50%, or at least about 60% of cells in said population of cells are said isolated CD73⁺, CD105⁺ placental cells. In another embodiment, at least about 70% of cells in said population of cells are said isolated CD73⁺, CD105⁺ placental cells. In another embodiment, at least about 90%, 95% or 99% of cells in said population of cells are said isolated CD73⁺, CD105⁺ placental cells. In a specific embodiment of the above populations, said isolated CD73⁺, CD105⁺ placental cells are additionally CD34⁻, CD38⁻ or CD45⁻. In another specific embodiment, said isolated CD73⁺, CD105⁺ placental cells are additionally CD34⁻, CD38⁻ and CD45⁻. In another specific embodiment, said isolated CD73⁺, CD105⁺ placental cells are additionally OCT-4⁺. In another specific embodiment, said isolated CD73⁺, CD105⁺ placental cells are additionally CD200⁺. In another specific embodiment, said isolated CD73⁺, CD105⁺ placental cells are additionally CD34⁻, CD38⁻, CD45⁻, OCT-4⁺ and CD200⁺. In another specific embodiment, said cell population is isolated away from placental cells that are not said isolated CD73⁺, CD105⁺ placental cells. In another specific embodiment, said cell population is isolated away from placental cells that do not display these markers.

In another embodiment, the isolated placental cells used to generate the stimulated PDACs described herein, which are useful in the methods and compositions described herein are OCT-4⁺ and facilitate formation of one or more embryoid-like bodies in a population of isolated placental cells comprising said cells when cultured under conditions that allow formation of embryoid-like bodies. In a specific embodiment, said isolated OCT-4⁺ placental cells are additionally CD73⁺ and CD105⁺. In another specific embodiment, said isolated OCT-4⁺ placental cells are additionally CD34⁻, CD38⁻, or CD45⁻. In another specific embodiment, said isolated OCT-4⁺ placental cells are additionally CD200⁺. In another specific embodiment, said isolated OCT-4⁺ placental cells are additionally CD73⁺, CD105⁺, CD200⁺, CD34⁻, CD38⁻, and CD45⁻. In another specific embodiment, said isolated OCT-4⁺ placental cells are isolated away from placental cells that are not OCT-4⁺ placental cells. In another specific embodiment, said isolated OCT-4⁺ placental cells are isolated away from placental cells that do not display these characteristics.

In another embodiment, a cell population used to generate the stimulated PDACs described herein, which are useful in the methods and compositions described herein is a population of cells comprising, e.g., a population that is enriched for isolated placental cells that are OCT-4⁺ and facilitate the formation of one or more embryoid-like bodies in a population of isolated placental cells comprising said cells when said population is cultured under conditions that allow formation of embryoid-like bodies. In various embodiments, at least about 10%, at least about 20%, at least about 30%, at least about 40%, at least about 50%, or at least about 60% of cells in said population of cells are said isolated OCT-4⁺ placental cells. In another embodiment, at least about 70% of cells in said population of cells are said isolated OCT-4⁺ placental cells. In another embodiment, at least about 80%, 90%, 95% or 99% of cells in said population of cells are said isolated OCT-4⁺ placental cells. In a specific embodiment of the above populations, said isolated OCT-4⁺ placental cells are additionally CD34⁻, CD38⁻ or CD45−. In another specific embodiment, said isolated OCT-4⁺ placental cells are additionally CD34⁻, CD38⁻ and CD45⁻. In another specific embodiment, said isolated OCT-4⁺ placental cells are additionally CD73⁺ and CD105⁺. In another specific embodiment, said isolated OCT-4⁺ placental cells are additionally CD200⁺. In another specific embodiment, said isolated OCT-4⁺ placental cells are additionally CD73⁺, CD105⁺, CD200⁺, CD34⁻, CD38⁻, and CD45⁻. In another specific embodiment, said cell population is isolated away from placental cells that are not said cells. In another specific embodiment, said cell population is isolated away from placental cells that do not display these markers.

In another embodiment, the isolated placental cells used to generate the stimulated PDACs described herein, which are useful in the methods and compositions described herein are isolated HLA-A,B,C⁺, CD45⁻, CD133⁻ and CD34⁻ placental cells. In another embodiment, a cell population used to generate the stimulated PDACs described herein, which are useful in the methods and compositions described herein is a population of cells comprising isolated placental cells, wherein at least about 70%, at least about 80%, at least about 90%, at least about 95% or at least about 99% of cells in said isolated population of cells are isolated HLA-A,B,C⁺, CD45⁻, CD133⁻ and CD34⁻ placental cells. In a specific embodiment, said isolated placental cell or population of isolated placental cells is isolated away from placental cells that are not HLA-A,B,C⁺, CD45⁻, CD133⁻ and CD34⁻ placental cells. In another specific embodiment, said isolated placental cells are non-maternal in origin. In another specific embodiment, said isolated population of placental cells are substantially free of maternal components; e.g., at least about 40%, 45%, 5-0%, 55%, 60%, 65%, 70%, 75%, 90%, 85%, 90%, 95%, 98% or 99% of said cells in said isolated population of placental cells are non-maternal in origin.

In another embodiment, the isolated placental cells used to generate the stimulated PDACs described herein, which are useful in the methods and compositions described herein are isolated CD10⁺, CD13⁺, CD33⁺, CD45⁻, CD11T and CD133⁻ placental cells. In another embodiment, a cell population useful in the methods and compositions described herein is a population of cells comprising isolated placental cells, wherein at least about 70%, at least about 80%, at least about 90%, at least about 95% or at least about 99% of cells in said population of cells are isolated CD10⁺, CD13⁺, CD33⁺, CD45⁻, CD11T and CD133⁻ placental cells. In a specific embodiment, said isolated placental cells or population of isolated placental cells is isolated away from placental cells that are not said isolated placental cells. In another specific embodiment, said isolated CD10⁺, CD13⁺, CD33⁺, CD45⁻, CD11T and CD133⁻ placental cells are non-maternal in origin, i.e., have the fetal genotype. In another specific embodiment, at least about 40%, 45%, 50%, 55%, 60%, 65%, 70%, 75%, 90%, 85%, 90%, 95%, 98% or 99% of said cells in said isolated population of placental cells, are non-maternal in origin. In another specific embodiment, said isolated placental cells or population of isolated placental cells are isolated away from placental cells that do not display these characteristics.

In another embodiment, the isolated placental cells used to generate the stimulated PDACs described herein, which are useful in the methods and compositions described herein are isolated CD10⁻, CD33⁻, CD44⁺, CD45⁻, and CD11T placental cells. In another embodiment, a cell population useful for the in the methods and compositions described herein is a population of cells comprising, e.g., a population enriched for isolated placental cells, wherein at least about 70%, at least about 80%, at least about 90%, at least about 95% or at least about 99% of cells in said population of cells are isolated CD10⁻, CD33⁻, CD44⁺, CD45⁻, and CD11T placental cells. In a specific embodiment, said isolated placental cell or population of isolated placental cells is isolated away from placental cells that are not said cells. In another specific embodiment, said isolated placental cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental cell or population of isolated placental cells is isolated away from placental cells that do not display these markers.

In another embodiment, the isolated placental cells used to generate the stimulated PDACs described herein, which are useful in the methods and compositions described herein are isolated CD10⁻, CD13⁻, CD33⁻, CD45⁻, and CD117-placental cells. In another embodiment, a cell population useful in the methods and compositions described herein is a population of cells comprising, e.g., a population enriched for isolated CD10⁻, CD13⁻, CD33⁻, CD45⁻, and CD11T placental cells, wherein at least about 70%, at least about 80%, at least about 90%, at least about 95% or at least about 99% of cells in said population are CD10⁻, CD13⁻, CD33⁻, CD45⁻, and CD117⁺ placental cells. In a specific embodiment, said isolated placental cells or population of isolated placental cells are isolated away from placental cells that are not said cells. In another specific embodiment, said isolated placental cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental cells or population of isolated placental cells is isolated away from placental cells that do not display these characteristics.

In another embodiment, the isolated placental cells used to generate the stimulated PDACs described herein, which are useful in the methods and compositions described herein are HLA A,B,C⁺, CD45⁻, CD34⁻, and CD133⁻, and are additionally CD10⁺, CD13⁺, CD38⁺, CD44⁺, CD90⁺, CD105⁺, CD200⁺ and/or HLA-G⁻, and/or negative for CD117. In another embodiment, a cell population useful in the methods described herein is a population of cells comprising isolated placental cells, wherein at least about 20%, 25%, 30%, 35%, 40%, 45%, 50%, 55%, 60%, 65%, 70%, 75%, 80%, 85%, 90%, 95%, 98% or about 99% of the cells in said population are isolated placental cells that are HLA A,B,C⁻, CD45⁻, CD34⁻, CD133⁻, and that are additionally positive for CD10, CD13, CD38, CD44, CD90, CD105, CD200, and/or negative for CD117 and/or HLA-G. In a specific embodiment, said isolated placental cells or population of isolated placental cells are isolated away from placental cells that are not said cells. In another specific embodiment, said isolated placental cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental cells or population of isolated placental cells are isolated away from placental cells that do not display these markers.

In another embodiment, the isolated placental cells used to generate the stimulated PDACs described herein, which are useful in the methods and compositions described herein are isolated placental cells that are CD200⁺ and CD10⁺, as determined by antibody binding, and CD117⁻, as determined by both antibody binding and RT-PCR. In another embodiment, the isolated placental cells useful in the methods and compositions described herein are isolated placental cells, e.g., placental stem cells or placental multipotent cells, that are CD10⁺, CD29⁻, CD54⁺, CD200⁺, HLA-G⁻, MHC class I⁺ and β-2-microglobulin⁺. In another embodiment, isolated placental cells useful in the methods and compositions described herein are placental cells wherein the expression of at least one cellular marker is at least two-fold higher than for a mesenchymal stem cell (e.g., a bone marrow-derived mesenchymal stem cell). In another specific embodiment, said isolated placental cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin.

In another embodiment, the isolated placental cells used to generate the stimulated PDACs described herein, which are useful in the methods and compositions described herein are isolated placental cells, e.g., placental stem cells or placental multipotent cells, that are one or more of CD10⁺, CD29⁺, CD44⁺, CD45⁻, CD54/ICAM⁺, CD62E⁻, CD62L⁻, CD62P⁻, CD80⁻, CD86⁻, CD103⁻, CD104⁻, CD105⁺, CD106/VCAM⁺, CD144/VE-cadherin^{low}, CD184/CXCR4⁻, β2-microglobulin^{low}, MHC-I^{low}, MCH-II⁻, HLA-G^{low}, and/or PDL1^{low}. In a specific embodiment, the isolated placental cells are at least CD29⁺ and CD54⁺. In another specific embodiment, the isolated placental cells are at least CD44⁺ and CD106⁺. In another specific embodiment, the isolated placental cells are at least CD29⁺.

In another embodiment, a cell population used to generate the stimulated PDACs described herein, which are useful in the methods and compositions described herein comprises isolated placental cells, and at least 50%, 60%, 70%, 80%, 90%, 95%, 98% or 99% of the cells in said cell population are isolated placental cells that are one or more of CD10⁺, CD29⁺, CD44⁺, CD45⁻, CD54/ICAM⁺, CD62-E⁻, CD62-L⁻, CD62-P⁻, CD80⁻, CD86⁻, CD103⁻, CD104⁻, CD105⁺, CD106/VCAM⁺, CD144/VE-cadherin_{dim}, CD184/CXCR4⁻, 2-microglobulin_{dim}, HLA-I^{dim}, HLA-II⁻, HLA-G^{dim}, and/or PDL1^{dim}. In another specific embodiment, at least 50%, 60%, 70%, 80%, 90%, 95%, 98% or 99% of cells in said cell population are CD10⁺, CD29⁺, CD44⁺, CD45⁻, CD54/ICAM⁺, CD62-E⁻, CD62-L⁻, CD62-P⁻, CD80⁻, CD86⁻, CD103⁻, CD104⁻, CD105⁺, CD106/VCAM⁺, CD144/VE-cadherin^{dim}, CD184/CXCR4⁻, β2-microglobulin^{dim}, MHC-I^{dim}, MHC-II⁻, HLA-G^{dim}, and PDL1^{dim}.

In another embodiment, the isolated placental cells used to generate the stimulated PDACs described herein, which are useful in the methods and compositions described herein are isolated placental cells that are one or more, or all, of CD10⁺, CD29⁺, CD34⁻, CD38⁻, CD44⁺, CD45⁻, CD54⁺, CD90⁺, SH2⁺, SH3⁺, SH4⁺, SSEA3⁻, SSEA4⁻, OCT-4⁺, and ABC-p⁺, where ABC-p is a placenta-specific ABC transporter protein (also known as breast cancer resistance protein (BCRP) and as mitoxantrone resistance protein (MXR)), wherein said isolated placental cells are obtained by perfusion of a mammalian, e.g., human, placenta that has been drained of cord blood and perfused to remove residual blood.

In another specific embodiment of any of the above characteristics, expression of the cellular marker (e.g., cluster of differentiation or immunogenic marker) is determined by flow cytometry; in another specific embodiment, expression of the marker is determined by RT-PCR.

Gene profiling confirms that isolated placental cells, and populations of isolated placental cells (e.g., the isolated placental cells and populations of isolated placental cells used to generate the stimulated PDACs described herein), are distinguishable from other cells, e.g., mesenchymal stem cells, e.g., bone marrow-derived mesenchymal stem cells. The isolated placental cells described herein can be distinguished from, e.g., mesenchymal stem cells on the basis of the expression of one or more genes, the expression of which is significantly higher in the isolated placental cells, or in certain isolated umbilical cord stem cells, in comparison to bone marrow-derived mesenchymal stem cells. In particular, the isolated placental cells used to generate the stimulated PDACs described herein, which are useful in the methods of treatment provided herein, can be distinguished from mesenchymal stem cells on the basis of the expression of one or more genes, the expression of which is significantly higher (that is, at least twofold higher) in the isolated placental cells than in an equivalent number of bone marrow-derived mesenchymal stem cells, wherein the one or more genes are ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRC5B, HLA-G, ICAM1, IER3, IGFBP7, IL1A, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, ZC3H12A, or a combination of any of the foregoing, when the cells are grown under equivalent conditions. See, e.g., U.S. Patent Application Publication No. 2007/0275362, the disclosure of which is incorporated herein by reference in its entirety. In certain specific embodiments, said expression of said one or more genes is determined, e.g., by RT-PCR or microarray analysis, e.g, using a U133-A microarray (Affymetrix). In another specific embodiment, said isolated placental cells express said one or more genes when cultured for a number of population doublings, e.g., anywhere from about 3 to about 35 population doublings, in a medium comprising DMEM-LG (e.g., from Gibco); 2% fetal calf serum (e.g., from Hyclone Labs.); lx insulin-transferrin-selenium (ITS); lx linoleic acid-bovine serum albumin (LA-BSA); 10⁻⁹ M dexamethasone (e.g., from Sigma); 10⁻⁴ M ascorbic acid 2-phosphate (e.g., from Sigma); epidermal growth factor 10 ng/mL (e.g., from R&D Systems); and platelet-derived growth factor (PDGF-BB) 10 ng/mL (e.g., from R&D Systems). In another specific embodiment, the isolated placental cell-specific or isolated umbilical cord cell-specific gene is CD200.

Specific sequences for these genes can be found in GenBank at accession nos. NM_001615 (ACTG2), BC065545 (ADARB1), (NM_181847 (AMIGO2), AY358590 (ARTS-1), BC074884 (B4GALT6), BC008396 (BCHE), BC020196 (C11orf9), BC031103 (CD200), NM_001845 (COL4A1), NM_001846 (COL4A2), BC052289 (CPA4), BC094758 (DMD), AF293359 (DSC3), NM_001943 (DSG2), AF338241 (ELOVL2), AY336105 (F2RL1), NM_018215 (FLJ10781), AY416799 (GATA6), BC075798 (GPR126), NM_016235 (GPRC5B), AF340038 (ICAM1), BC000844 (IER3), BC066339 (IGFBP7), BC013142 (IL1A), BT019749 (IL6), BC007461 (IL18), (BC072017) KRT18, BC075839 (KRT8), BC060825 (LIPG), BC065240 (LRAP), BC010444 (MATN2), BC011908 (MEST), BC068455 (NFE2L3), NM_014840 (NUAK1), AB006755 (PCDH7), NM_014476 (PDLIM3), BC126199 (PKP-2), BC090862 (RTN1), BC002538 (SERPINB9), BC023312 (ST3GAL6), BC001201 (ST6GALNAC5), BC126160 or BC065328 (SLC12A8), BC025697 (TCF21), BC096235 (TGFB2), BC005046 (VTN), and BC005001 (ZC3H12A) as of March 2008.

In certain specific embodiments, said isolated placental cells used to generate the stimulated PDACs described herein express each of ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRC5B, HLA-G, ICAM1, IER3, IGFBP7, IL1A, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, and ZC3H12A at a detectably higher level than an equivalent number of bone marrow-derived mesenchymal stem cells, when the cells are grown under equivalent conditions.

In specific embodiments, the placental cells used to generate the stimulated PDACs described herein express CD200 and ARTS1 (aminopeptidase regulator of type 1 tumor necrosis factor); ARTS-1 and LRAP (leukocyte-derived arginine aminopeptidase); IL6 (interleukin-6) and TGFB2 (transforming growth factor, beta 2); IL6 and KRT18 (keratin 18); IER3 (immediate early response 3), MEST (mesoderm specific transcript homolog) and TGFB2; CD200 and IER3; CD200 and IL6; CD200 and KRT18; CD200 and LRAP; CD200 and MEST; CD200 and NFE2L3 (nuclear factor (erythroid-derived 2)-like 3); or CD200 and TGFB2 at a detectably higher level than an equivalent number of bone marrow-derived mesenchymal stem cells (BM-MSCs) wherein said bone marrow-derived mesenchymal stem cells have undergone a number of passages in culture equivalent to the number of passages said isolated placental cells have undergone. In other specific embodiments, the placental cells used to generate the stimulated PDACs described herein express ARTS-1, CD200, IL6 and LRAP; ARTS-1, IL6, TGFB2, IER3, KRT18 and MEST; CD200, IER3, IL6, KRT18, LRAP, MEST, NFE2L3, and TGFB2; ARTS-1, CD200, IER3, IL6, KRT18, LRAP, MEST, NFE2L3, and TGFB2; or IER3, MEST and TGFB2 at a detectably higher level than an equivalent number of bone marrow-derived mesenchymal stem cells BM-MSCs, wherein said bone marrow-derived mesenchymal stem cells have undergone a number of passages in culture equivalent to the number of passages said isolated placental cells have undergone.

Expression of the above-referenced genes can be assessed by standard techniques. For example, probes based on the sequence of the gene(s) can be individually selected and constructed by conventional techniques. Expression of the genes can be assessed, e.g., on a microarray comprising probes to one or more of the genes, e.g., an Affymetrix GENECHIP® Human Genome U133A 2.0 array, or an Affymetrix GENECHIP® Human Genome U133 Plus 2.0 (Santa Clara, Calif.). Expression of these genes can be assessed even if the sequence for a particular GenBank accession number is amended because probes specific for the amended sequence can readily be generated using well-known standard techniques.

The level of expression of these genes can be used to confirm the identity of a population of isolated placental cells, to identify a population of cells as comprising at least a plurality of isolated placental cells, or the like. Populations of isolated placental cells, the identity of which is confirmed, can be clonal, e.g., populations of isolated placental cells expanded from a single isolated placental cell, or a mixed population of stem cells, e.g., a population of cells comprising solely isolated placental cells that are expanded from multiple isolated placental cells, or a population of cells comprising isolated placental cells, as described herein, and at least one other type of cell.

The level of expression of these genes can be used to select populations of isolated placental cells. For example, a population of cells, e.g., clonally-expanded cells, may be selected if the expression of one or more of the genes listed above is significantly higher in a sample from the population of cells than in an equivalent population of mesenchymal stem cells. Such selecting can be of a population from a plurality of isolated placental cell populations, from a plurality of cell populations, the identity of which is not known, etc.

Isolated placental cells used to generate the stimulated PDACs described herein can be selected on the basis of the level of expression of one or more such genes as compared to the level of expression in said one or more genes in, e.g., a mesenchymal stem cell control, for example, the level of expression in said one or more genes in an equivalent number of bone marrow-derived mesenchymal stem cells. In one embodiment, the level of expression of said one or more genes in a sample comprising an equivalent number of mesenchymal stem cells is used as a control. In another embodiment, the control, for isolated placental cells used to generate the stimulated PDACs described herein tested under certain conditions, is a numeric value representing the level of expression of said one or more genes in mesenchymal stem cells under said conditions.

In certain embodiments, the placental cells (e.g., PDACs) useful in the methods provided herein, do not express CD34, as detected by immunolocalization, after exposure to 1 to 100 ng/mL VEGF for 4 to 21 days. In a specific embodiment, said placental adherent cells are adherent to tissue culture plastic. In another specific embodiment, said population of cells induce endothelial cells to form sprouts or tube-like structures when cultured in the presence of an angiogenic factor such as vascular endothelial growth factor (VEGF), epithelial growth factor (EGF), platelet derived growth factor (PDGF) or basic fibroblast growth factor (bFGF), e.g., on a substrate such as MATRIGEL™.

In another aspect, the placental cells used to generate the stimulated PDACs described herein, a population of cells, e.g., a population of PDACs, or a population of cells wherein at least about 50%, 60%, 70%, 80%, 90%, 95% or 98% of cells in said isolated population of cells are PDACs, secrete one or more, or all, of VEGF, HGF, IL-8, MCP-3, FGF2, follistatin, G-CSF, EGF, ENA-78, GRO, IL-6, MCP-1, PDGF-BB, TMP-2, uPAR, or galectin-1, e.g., into culture medium in which the cell, or cells, are grown. In another embodiment, the placental cells used to generate the stimulated PDACs described herein, express increased levels of CD202b, IL-8 and/or VEGF under hypoxic conditions (e.g., less than about 5% 02) compared to normoxic conditions (e.g., about 20% or about 21% O₂).

In another embodiment, any of the placental cells or populations of cells comprising the placental cells used to generate the stimulated PDACs described herein can cause the formation of sprouts or tube-like structures in a population of endothelial cells in contact with said placental derived adherent cells. In a specific embodiment, the placental cells used to generate the stimulated PDACs described herein, are co-cultured with human endothelial cells, which form sprouts or tube-like structures, or support the formation of endothelial cell sprouts, e.g., when cultured in the presence of extracellular matrix proteins such as collagen type I and IV, and/or angiogenic factors such as vascular endothelial growth factor (VEGF), epithelial growth factor (EGF), platelet derived growth factor (PDGF) or basic fibroblast growth factor (bFGF), e.g., in or on a substrate such as placental collagen or MATRIGEL™ fora t least 4 days. In another embodiment, any of the populations of cells comprising placental derived adherent cells, described herein, secrete angiogenic factors such as vascular endothelial growth factor (VEGF), hepatocyte growth factor (HGF), platelet derived growth factor (PDGF), basic fibroblast growth factor (bFGF), or Interleukin-8 (IL-8) and thereby can induce human endothelial cells to form sprouts or tube-like structures when cultured in the presence of extracellular matrix proteins such as collagen type I and IV e.g., in or on a substrate such as placental collagen or MATRIGEL™.

In another embodiment, any of the above populations of cells comprising placental derived adherent cells (PDACs) secretes angiogenic factors. In specific embodiments, the population of cells secretes vascular endothelial growth factor (VEGF), hepatocyte growth factor (HGF), platelet derived growth factor (PDGF), basic fibroblast growth factor (bFGF), and/or interleukin-8 (IL-8). In other specific embodiments, the population of cells comprising PDACs secretes one or more angiogenic factors and thereby induces human endothelial cells to migrate in an in vitro wound healing assay. In other specific embodiments, the population of cells comprising placental derived adherent cells induces maturation, differentiation or proliferation of human endothelial cells, endothelial progenitors, myocytes or myoblasts.

The isolated placental cells described herein display the above characteristics (e.g., combinations of cell surface markers and/or gene expression profiles) in primary culture, or during proliferation in medium comprising, e.g., DMEM-LG (Gibco), 2% fetal calf serum (FCS) (Hyclone Laboratories), lx insulin-transferrin-selenium (ITS), 1× lenolenic-acid-bovine-serum-albumin (LA-BSA), 10⁻⁹M dexamethasone (Sigma), 10⁻⁴M ascorbic acid 2-phosphate (Sigma), epidermal growth factor (EGF) 10 ng/ml (R&D Systems), platelet derived-growth factor (PDGF-BB) 10 ng/ml (R&D Systems), and 100 U penicillin/1000 U streptomycin.

In certain embodiments of any of the placental cells used to generate the stimulated PDACs described herein, the cells are human. In certain embodiments of any of the placental cells used to generate the stimulated PDACs described herein, the cellular marker characteristics or gene expression characteristics are human markers or human genes.

In another specific embodiment of said isolated placental cells or populations of cells used to generate the stimulated PDACs described herein, said cells or population have been expanded, for example, passaged at least, about, or no more than, 1, 2, 3, 4, 5, 6, 7, 8, 9, 10, 11, 12, 13, 14, 15, 16, 17, 18, 19, or 20 times, or more, or proliferated for at least, about, or no more than, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 population doublings. In another specific embodiment of said isolated placental cells or populations of cells used to generate the stimulated PDACs described herein, said cells or population are primary isolates. In another specific embodiment of the isolated placental cells, or populations of cells comprising isolated placental cells, that are disclosed herein, said isolated placental cells are fetal in origin (that is, have the fetal genotype).

In certain embodiments, said isolated placental cells used to generate the stimulated PDACs described herein, do not differentiate during culturing in growth medium, i.e., medium formulated to promote proliferation, e.g., during proliferation in growth medium. In another specific embodiment, said isolated placental cells do not require a feeder layer in order to proliferate. In another specific embodiment, said isolated placental cells do not differentiate in culture in the absence of a feeder layer, solely because of the lack of a feeder cell layer.

In another embodiment, cells used to generate the stimulated PDACs described herein, which are useful in the methods and compositions described herein are isolated placental cells, wherein a plurality of said isolated placental cells are positive for aldehyde dehydrogenase (ALDH), as assessed by an aldehyde dehydrogenase activity assay. Such assays are known in the art (see, e.g., Bostian and Betts, Biochem. J., 173, 787, (1978)). In a specific embodiment, said ALDH assay uses ALDEFLUOR® (Aldagen, Inc., Ashland, Oreg.) as a marker of aldehyde dehydrogenase activity. In a specific embodiment, said plurality is between about 3% and about 25% of cells in said population of cells. In another embodiment, provided herein is a population of isolated umbilical cord cells, e.g., multipotent isolated umbilical cord cells, wherein a plurality of said isolated umbilical cord cells are positive for aldehyde dehydrogenase, as assessed by an aldehyde dehydrogenase activity assay that uses ALDEFLUOR® as an indicator of aldehyde dehydrogenase activity. In a specific embodiment, said plurality is between about 3% and about 25% of cells in said population of cells. In another embodiment, said population of isolated placental cells or isolated umbilical cord cells shows at least three-fold, or at least five-fold, higher ALDH activity than a population of bone marrow-derived mesenchymal stem cells having about the same number of cells and cultured under the same conditions.

In certain embodiments of any of the populations of cells comprising the isolated placental cells used to generate the stimulated PDACs described herein, the placental cells in said populations of cells are substantially free of cells having a maternal genotype; e.g., at least 40%, 45%, 50%, 55%, 60%, 65%, 70%, 75%, 80%, 85%, 90%, 95%, 98% or 99% of the placental cells in said population have a fetal genotype. In certain other embodiments of any of the populations of cells comprising the isolated placental cells described herein, the populations of cells comprising said placental cells are substantially free of cells having a maternal genotype; e.g., at least 40%, 45%, 50%, 55%, 60%, 65%, 70%, 75%, 80%, 85%, 90%, 95%, 98% or 99% of the cells in said population have a fetal genotype.

In a specific embodiment of any of the above isolated placental cells or cell populations used to generate the stimulated PDACs described herein, the karyotype of the cells, or at least about 95% or about 99% of the cells in said population, is normal. In another specific embodiment of any of the above placental cells or cell populations, the cells, or cells in the population of cells, are non-maternal in origin.

Isolated placental cells, or populations of isolated placental cells used to generate the stimulated PDACs described herein, bearing any of the above combinations of markers, can be combined in any ratio. Any two or more of the above isolated placental cell populations can be combined to form an isolated placental cell population. For example, an population of isolated placental cells can comprise a first population of isolated placental cells defined by one of the marker combinations described above, and a second population of isolated placental cells defined by another of the marker combinations described above, wherein said first and second populations are combined in a ratio of about 1:99, 2:98, 3:97, 4:96, 5:95, 10:90, 20:80, 30:70, 40:60, 50:50, 60:40, 70:30, 80:20, 90:10, 95:5, 96:4, 97:3, 98:2, or about 99:1. In like fashion, any three, four, five or more of the above-described isolated placental cells or isolated placental cells populations can be combined.

Isolated placental cells used to generate the stimulated PDACs described herein, which are useful in the methods and compositions described herein can be obtained, e.g., by disruption of placental tissue, with or without enzymatic digestion (see Section 5.3.3) or perfusion (see Section 5.3.4). For example, populations of isolated placental cells can be produced according to a method comprising perfusing a mammalian placenta that has been drained of cord blood and perfused to remove residual blood; perfusing said placenta with a perfusion solution; and collecting said perfusion solution, wherein said perfusion solution after perfusion comprises a population of placental cells that comprises isolated placental cells; and isolating a plurality of said isolated placental cells from said population of cells. In a specific embodiment, the perfusion solution is passed through both the umbilical vein and umbilical arteries and collected after it exudes from the placenta. In another specific embodiment, the perfusion solution is passed through the umbilical vein and collected from the umbilical arteries, or passed through the umbilical arteries and collected from the umbilical vein.

In various embodiments, the isolated placental cells used to generate the stimulated PDACs described herein, contained within a population of cells obtained from perfusion of a placenta, are at least 50%, 60%, 70%, 80%, 90%, 95%, 99% or at least 99.5% of said population of placental cells. In another specific embodiment, the isolated placental cells collected by perfusion comprise fetal and maternal cells. In another specific embodiment, the isolated placental cells collected by perfusion are at least 50%, 60%, 70%, 80%, 90%, 95%, 99% or at least 99.5% fetal cells.

In another specific embodiment, provided herein is a composition comprising a population of the isolated placental cells used to generate the stimulated PDACs described herein, collected by perfusion, wherein said composition comprises at least a portion of the perfusion solution used to collect the isolated placental cells.

Isolated populations of the isolated placental cells used to generate the stimulated PDACs described herein can be produced by digesting placental tissue with a tissue-disrupting enzyme to obtain a population of placental cells comprising the cells, and isolating, or substantially isolating, a plurality of the placental cells from the remainder of said placental cells. The whole, or any part of, the placenta can be digested to obtain the isolated placental cells described herein. In specific embodiments, for example, said placental tissue can be a whole placenta, an amniotic membrane, chorion, a combination of amnion and chorion, or a combination of any of the foregoing. In other specific embodiment, the tissue-disrupting enzyme is trypsin or collagenase. In various embodiments, the isolated placental cells, contained within a population of cells obtained from digesting a placenta, are at least 50%, 60%, 70%, 80%, 90%, 95%, 99% or at least 99.5% of said population of placental cells.

The isolated populations of placental cells described above, and populations of isolated placental cells used to generate the stimulated PDACs described herein, generally can comprise about, at least, or no more than, 1×10², 5×10² 1×10³, 5×10³, 1×10⁴, 5×10⁴′ 1×10⁵, 5×10⁵, 1×10⁶, 5×10⁶, 1×10⁷, 5×10⁷, 1×10⁸, 5×10⁸, 1×10⁹, 5×10⁹, 1×10¹°, 5×10¹°, 1×10¹¹ or more of the isolated placental cells. Populations of isolated placental cells useful in the methods of treatment described herein comprise at least 50%, 55%, 60%, 65%, 70%, 75%, 80%, 85%, 90%, 95%, 98%, or 99% viable isolated placental cells, e.g., as determined by, e.g., trypan blue exclusion.

### 5.2.3 Growth in Culture

The growth of the isolated placental cells described herein in Section 5.2, e.g., the placental cells used to generate the stimulated PDACs described herein, as for any mammalian cell, depends in part upon the particular medium selected for growth. Under optimum conditions, the isolated placental cells typically double in number in about 1 day. During culture, the isolated placental cells described herein adhere to a substrate in culture, e.g. the surface of a tissue culture container (e.g., tissue culture dish plastic, fibronectin-coated plastic, and the like) and form a monolayer.

Populations of placental cells that comprise the isolated placental cells used to generate the stimulated PDACs described herein, when cultured under appropriate conditions, can form embryoid-like bodies, that is, three-dimensional clusters of cells grow atop the adherent cell layer. Cells within the embryoid-like bodies can express markers associated with very early stem cells, e.g., OCT-4, Nanog, SSEA3 and SSEA4. Cells within the embryoid-like bodies are typically not adherent to the culture substrate, as are the isolated placental cells described herein, but tend to remain attached to the adherent cells during culture. Embryoid-like body cells are dependent upon the adherent isolated placental cells for viability, as embryoid-like bodies do not form in the absence of the adherent isolated placental cells. The adherent isolated placental cells thus facilitate the growth of one or more embryoid-like bodies in a population of placental cells that comprise the adherent isolated placental cells. Without wishing to be bound by theory, the cells of the embryoid-like bodies are thought to grow on the adherent isolated placental cells much as embryonic stem cells grow on a feeder layer of cells.

### 5.3 Methods of Obtaining Isolated Placental Cells

### 5.3.1 Stem Cell Collection Composition

Further provided herein are methods of collecting and isolating placental cells, e.g., the isolated placental cells described in Section 5.2.2, above, for example the isolated placental cells used to generate the stimulated PDACs described herein. Generally, such cells are obtained from a mammalian placenta using a physiologically-acceptable solution, e.g., a cell collection composition. An exemplary cell collection composition is described in detail in related U.S. Patent Application Publication No. 2007/0190042, entitled “Improved Medium for Collecting Placental Stem Cells and Preserving Organs,” the disclosure of which is incorporated herein by reference in its entirety

The cell collection composition can comprise any physiologically-acceptable solution suitable for the collection and/or culture of cells, e.g., the isolated placental cells described herein, for example, a saline solution (e.g., phosphate-buffered saline, Kreb's solution, modified Kreb's solution, Eagle's solution, 0.9% NaCl. etc.), a culture medium (e.g., DMEM, H.DMEM, etc.), and the like.

The cell collection composition can comprise one or more components that tend to preserve isolated placental cells, that is, prevent the isolated placental cells from dying, or delay the death of the isolated placental cells, reduce the number of isolated placental cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

The cell collection composition can comprise one or more tissue-degrading enzymes, e.g., a metalloprotease, a serine protease, a neutral protease, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE, hyaluronidase, and the like.

The cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like. In one embodiment, the antibiotic is gentamycin, e.g., about 0.005% to about 0.01% (w/v) in culture medium

The cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/1, or about 40 g/l to about 60 g/l); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/l to about 100,000 units/l); or an amiloride containing compound (e.g., amiloride, ethyl isopropyl amiloride, hexamethylene amiloride, dimethyl amiloride or isobutyl amiloride present at about 1.0 μM to about 5 μM).

### 5.3.2 Collection and Handling of Placenta

Generally, a human placenta is recovered shortly after its expulsion after birth. In a preferred embodiment, the placenta is recovered from a patient after informed consent and after a complete medical history of the patient is taken and is associated with the placenta. Preferably, the medical history continues after delivery. Such a medical history can be used to coordinate subsequent use of the placenta or the isolated placental cells harvested therefrom. For example, isolated human placental cells can be used, in light of the medical history, for personalized medicine for the infant associated with the placenta, or for parents, siblings or other relatives of the infant.

Prior to recovery of isolated placental cells, the umbilical cord blood and placental blood are preferably removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank USA, Cedar Knolls, N.J. Preferably, the placenta is gravity drained without further manipulation so as to minimize tissue disruption during cord blood recovery.

Typically, a placenta is transported from the delivery or birthing room to another location, e.g., a laboratory, for recovery of cord blood and collection of stem cells by, e.g., perfusion or tissue dissociation. The placenta is preferably transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in pending U.S. Pat. No. 7,147,626, the disclosure of which is incorporated by reference herein. Preferably, the placenta is delivered to the laboratory four to twenty-four hours following delivery. In certain embodiments, the proximal umbilical cord is clamped, preferably within 4-5 cm (centimeter) of the insertion into the placental disc prior to cord blood recovery. In other embodiments, the proximal umbilical cord is clamped after cord blood recovery but prior to further processing of the placenta.

The placenta, prior to cell collection, can be stored under sterile conditions and at either room temperature or at a temperature of 5° C. to 25° C. The placenta may be stored for a period of for a period of four to twenty-four hours, up to forty-eight hours, or longer than forty eight hours, prior to perfusing the placenta to remove any residual cord blood. In one embodiment, the placenta is harvested from between about zero hours to about two hours post-expulsion. The placenta is preferably stored in an anticoagulant solution at a temperature of 5° C. to 25° C. Suitable anticoagulant solutions are well known in the art. For example, a solution of heparin or warfarin sodium can be used. In a preferred embodiment, the anticoagulant solution comprises a solution of heparin (e.g., 1% w/w in 1:1000 solution). The exsanguinated placenta is preferably stored for no more than 36 hours before placental cells are collected.

The mammalian placenta or a part thereof, once collected and prepared generally as above, can be treated in any art-known manner, e.g., can be perfused or disrupted, e.g., digested with one or more tissue-disrupting enzymes, to obtain isolated placental cells.

### 5.3.3 Physical Disruption and Enzymatic Digestion of Placental Tissue

In one embodiment, stem cells are collected from a mammalian placenta by physical disruption of part of all of the organ. For example, the placenta, or a portion thereof, may be, e.g., crushed, sheared, minced, diced, chopped, macerated or the like. The tissue can then be cultured to obtain a population of isolated placental cells. Typically, the placental tissue is disrupted using, e.g., culture medium, a saline solution, or a stem cell collection composition (see Section 5.5.1 and below).

The placenta can be dissected into components prior to physical disruption and/or enzymatic digestion and stem cell recovery. Isolated placental cells can be obtained from all or a portion of the amniotic membrane, chorion, umbilical cord, placental cotyledons, or any combination thereof, including from a whole placenta. Preferably, isolated placental cells are obtained from placental tissue comprising amnion and chorion. Typically, isolated placental cells can be obtained by disruption of a small block of placental tissue, e.g., a block of placental tissue that is about 1, 2, 3, 4, 5, 6, 7, 8, 9, 10, 20, 30, 40, 50, 60, 70, 80, 90, 100, 200, 300, 400, 500, 600, 700, 800, 900 or about 1000 cubic millimeters in volume. Any method of physical disruption can be used, provided that the method of disruption leaves a plurality, more preferably a majority, and more preferably at least 60%, 70%, 80%, 90%, 95%, 98%, or 99% of the cells in said organ viable, as determined by, e.g., trypan blue exclusion.

The isolated adherent placental cells can generally be collected from a placenta, or portion thereof, at any time within about the first three days post-expulsion, but preferably between about 8 hours and about 18 hours post-expulsion.

In a specific embodiment, the disrupted tissue is cultured in tissue culture medium suitable for the proliferation of isolated placental cells (see, e.g., Section 5.6, below, describing the culture of placental cells, e.g., PDACs).

In another specific embodiment, isolated placental cells are collected by physical disruption of placental tissue, wherein the physical disruption includes enzymatic digestion, which can be accomplished by use of one or more tissue-digesting enzymes. The placenta, or a portion thereof, may also be physically disrupted and digested with one or more enzymes, and the resulting material then immersed in, or mixed into, a cell collection composition.

A preferred cell collection composition comprises one or more tissue-disruptive enzyme(s). Enzymes that can be used to disrupt placenta tissue include papain, deoxyribonucleases, serine proteases, such as trypsin, chymotrypsin, collagenase, dispase or elastase. Serine proteases may be inhibited by alpha 2 microglobulin in serum and therefore the medium used for digestion is usually serum-free. EDTA and DNase are commonly used in enzyme digestion procedures to increase the efficiency of cell recovery. The digestate is preferably diluted so as to avoid trapping cells within the viscous digest.

Any combination of tissue digestion enzymes can be used. Typical concentrations for digestion using trypsin include, 0.1% to about 2% trypsin, e.g., about 0.25% trypsin. Proteases can be used in combination, that is, two or more proteases in the same digestion reaction, or can be used sequentially in order to liberate placental cells, e.g., placental stem cells and placental multipotent cells. For example, in one embodiment, a placenta, or part thereof, is digested first with an appropriate amount of collagenase I at about 1 to about 2 mg/ml for, e.g., 30 minutes, followed by digestion with trypsin, at a concentration of about 0.25%, for, e.g., 10 minutes, at 37° C. Serine proteases are preferably used consecutively following use of other enzymes.

In another embodiment, the tissue can further be disrupted by the addition of a chelator, e.g., ethylene glycol bis(2-aminoethyl ether)-N,N,N′N′-tetraacetic acid (EGTA) or ethylenediaminetetraacetic acid (EDTA) to the stem cell collection composition comprising the stem cells, or to a solution in which the tissue is disrupted and/or digested prior to isolation of the stem cells with the stem cell collection composition.

Following digestion, the digestate is washed, for example, three times with culture medium, and the washed cells are seeded into culture flasks. The cells are then isolated by differential adherence, and characterized for, e.g., viability, cell surface markers, differentiation, and the like.

It will be appreciated that where an entire placenta, or portion of a placenta comprising both fetal and maternal cells (for example, where the portion of the placenta comprises the chorion or cotyledons), the placental cells isolated can comprise a mix of placental cells derived from both fetal and maternal sources. Where a portion of the placenta that comprises no, or a negligible number of, maternal cells (for example, amnion), the placental cells isolated therefrom will comprise almost exclusively fetal placental cells (that is, placental cells having the genotype of the fetus).

Placental cells, e.g., the placental cells described in Section 5.2.2, above, can be isolated from disrupted placental tissue by differential trypsinization (see Section 5.3.5, below) followed by culture in one or more new culture containers in fresh proliferation medium, optionally followed by a second differential trypsinization step.

### 5.3.4 Placental Perfusion

Placental cells, e.g., the placental cells described in Section 5.2.2, above, for example the placental cells used to generate the stimulated PDACs described herein can also be obtained by perfusion of the mammalian placenta. Methods of perfusing mammalian placenta to obtain placental cells are disclosed, e.g., in Hariri, U.S. Pat. Nos. 7,045,148 and 7,255,729, in U.S. Patent Application Publication Nos. 2007/0275362 and 2007/0190042, the disclosures of each of which are incorporated herein by reference in their entireties.

Placental cells can be collected by perfusion, e.g., through the placental vasculature, using, e.g., a cell collection composition as a perfusion solution. In one embodiment, a mammalian placenta is perfused by passage of perfusion solution through either or both of the umbilical artery and umbilical vein. The flow of perfusion solution through the placenta may be accomplished using, e.g., gravity flow into the placenta. Preferably, the perfusion solution is forced through the placenta using a pump, e.g., a peristaltic pump. The umbilical vein can be, e.g., cannulated with a cannula, e.g., a TEFLON® or plastic cannula, that is connected to a sterile connection apparatus, such as sterile tubing. The sterile connection apparatus is connected to a perfusion manifold.

In preparation for perfusion, the placenta is preferably oriented (e.g., suspended) in such a manner that the umbilical artery and umbilical vein are located at the highest point of the placenta. The placenta can be perfused by passage of a perfusion fluid through the placental vasculature and surrounding tissue. The placenta can also be perfused by passage of a perfusion fluid into the umbilical vein and collection from the umbilical arteries, or passage of a perfusion fluid into the umbilical arteries and collection from the umbilical vein.

In one embodiment, for example, the umbilical artery and the umbilical vein are connected simultaneously, e.g., to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. Placental cells that are collected by this method, which can be referred to as a “pan” method, are typically a mixture of fetal and maternal cells.

In another embodiment, the perfusion solution is passed through the umbilical veins and collected from the umbilical artery, or is passed through the umbilical artery and collected from the umbilical veins. Placental cells collected by this method, which can be referred to as a “closed circuit” method, are typically almost exclusively fetal.

It will be appreciated that perfusion using the pan method, that is, whereby perfusate is collected after it has exuded from the maternal side of the placenta, results in a mix of fetal and maternal cells. As a result, the cells collected by this method can comprise a mixed population of placental cells, e.g., placental stem cells or placental multipotent cells, of both fetal and maternal origin. In contrast, perfusion solely through the placental vasculature in the closed circuit method, whereby perfusion fluid is passed through one or two placental vessels and is collected solely through the remaining vessel(s), results in the collection of a population of placental cells almost exclusively of fetal origin.

The closed circuit perfusion method can, in one embodiment, be performed as follows. A post-partum placenta is obtained within about 48 hours after birth. The umbilical cord is clamped and cut above the clamp. The umbilical cord can be discarded, or can processed to recover, e.g., umbilical cord stem cells, and/or to process the umbilical cord membrane for the production of a biomaterial. The amniotic membrane can be retained during perfusion, or can be separated from the chorion, e.g., using blunt dissection with the fingers. If the amniotic membrane is separated from the chorion prior to perfusion, it can be, e.g., discarded, or processed, e.g., to obtain stem cells by enzymatic digestion, or to produce, e.g., an amniotic membrane biomaterial, e.g., the biomaterial described in U.S. Application Publication No. 2004/0048796, the disclosure of which is incorporated by reference herein in its entirety. After cleaning the placenta of all visible blood clots and residual blood, e.g., using sterile gauze, the umbilical cord vessels are exposed, e.g., by partially cutting the umbilical cord membrane to expose a cross-section of the cord. The vessels are identified, and opened, e.g., by advancing a closed alligator clamp through the cut end of each vessel. The apparatus, e.g., plastic tubing connected to a perfusion device or peristaltic pump, is then inserted into each of the placental arteries. The pump can be any pump suitable for the purpose, e.g., a peristaltic pump. Plastic tubing, connected to a sterile collection reservoir, e.g., a blood bag such as a 250 mL collection bag, is then inserted into the placental vein. Alternatively, the tubing connected to the pump is inserted into the placental vein, and tubes to a collection reservoir(s) are inserted into one or both of the placental arteries. The placenta is then perfused with a volume of perfusion solution, e.g., about 750 ml of perfusion solution. Cells in the perfusate are then collected, e.g., by centrifugation. In certain embodiments, the placenta is perfused with perfusion solution, e.g., 100-300 mL perfusion solution, to remove residual blood prior to perfusion to collect placental cells, e.g., placental stem cells and/or placental multipotent cells. In another embodiment, the placenta is not perfused with perfusion solution to remove residual blood prior to perfusion to collect placental cells.

In one embodiment, the proximal umbilical cord is clamped during perfusion, and more preferably, is clamped within 4-5 cm (centimeter) of the cord's insertion into the placental disc.

The first collection of perfusion fluid from a mammalian placenta during the exsanguination process is generally colored with residual red blood cells of the cord blood and/or placental blood. The perfusion fluid becomes more colorless as perfusion proceeds and the residual cord blood cells are washed out of the placenta. Generally from 30 to 100 ml (milliliter) of perfusion fluid is adequate to initially exsanguinate the placenta, but more or less perfusion fluid may be used depending on the observed results.

The volume of perfusion liquid used to isolate placental cells may vary depending upon the number of cells to be collected, the size of the placenta, the number of collections to be made from a single placenta, etc. In various embodiments, the volume of perfusion liquid may be from 50 mL to 5000 mL, 50 mL to 4000 mL, 50 mL to 3000 mL, 100 mL to 2000 mL, 250 mL to 2000 mL, 500 mL to 2000 mL, or 750 mL to 2000 mL. Typically, the placenta is perfused with 700-800 mL of perfusion liquid following exsanguination.

The placenta can be perfused a plurality of times over the course of several hours or several days. Where the placenta is to be perfused a plurality of times, it may be maintained or cultured under aseptic conditions in a container or other suitable vessel, and perfused with the cell collection composition, or a standard perfusion solution (e.g., a normal saline solution such as phosphate buffered saline (“PBS”)) with or without an anticoagulant (e.g., heparin, warfarin sodium, coumarin, bishydroxycoumarin), and/or with or without an antimicrobial agent (e.g., 0-mercaptoethanol (0.1 mM); antibiotics such as streptomycin (e.g., at 40-100 μg/ml), penicillin (e.g., at 40 U/ml), amphotericin B (e.g., at 0.5 μg/ml). In one embodiment, an isolated placenta is maintained or cultured for a period of time without collecting the perfusate, such that the placenta is maintained or cultured for 1, 2, 3, 4, 5, 6, 7, 8, 9, 10, 11, 12, 13, 14, 15, 16, 17, 18, 19, 20, 21, 22, 23, or 24 hours, or 2 or 3 or more days before perfusion and collection of perfusate. The perfused placenta can be maintained for one or more additional time(s), e.g., 1, 2, 3, 4, 5, 6, 7, 8, 9, 10, 11, 12, 13, 14, 15, 16, 17, 18, 19, 20, 21, 22, 23, 24 or more hours, and perfused a second time with, e.g., 700-800 mL perfusion fluid. The placenta can be perfused 1, 2, 3, 4, 5 or more times, for example, once every 1, 2, 3, 4, 5 or 6 hours. In a preferred embodiment, perfusion of the placenta and collection of perfusion solution, e.g., cell collection composition, is repeated until the number of recovered nucleated cells falls below 100 cells/ml. The perfusates at different time points can be further processed individually to recover time-dependent populations of cells, e.g., stem cells. Perfusates from different time points can also be pooled. In a preferred embodiment, placental cells are collected at a time or times between about 8 hours and about 18 hours post-expulsion.

Perfusion preferably results in the collection of significantly more placental cells than the number obtainable from a mammalian placenta not perfused with said solution, and not otherwise treated to obtain placental cells (e.g., by tissue disruption, e.g., enzymatic digestion). In this context, “significantly more” means at least 10% more. Perfusion yields significantly more placental cells than, e.g., the number of placental cells isolatable from culture medium in which a placenta, or portion thereof, has been cultured.

Placental cells can be isolated from placenta by perfusion with a solution comprising one or more proteases or other tissue-disruptive enzymes. In a specific embodiment, a placenta or portion thereof (e.g., amniotic membrane, amnion and chorion, placental lobule or cotyledon, umbilical cord, or combination of any of the foregoing) is brought to 25-37° C., and is incubated with one or more tissue-disruptive enzymes in 200 mL of a culture medium for 30 minutes. Cells from the perfusate are collected, brought to 4° C., and washed with a cold inhibitor mix comprising 5 mM EDTA, 2 mM dithiothreitol and 2 mM beta-mercaptoethanol. The placental cells are washed after several minutes with a cold (e.g., 4° C.) stem cell collection composition.

### 5.3.5 Isolation, Sorting, and Characterization of Placental Cells

The isolated placental cells, e.g., the cells described in Section 5.2.2, above, for example the isolated placental cells used to generate the stimulated PDACs described herein whether obtained by perfusion or physical disruption, e.g., by enzymatic digestion, can initially be purified from (i.e., be isolated from) other cells by Ficoll gradient centrifugation. Such centrifugation can follow any standard protocol for centrifugation speed, etc. In one embodiment, for example, cells collected from the placenta are recovered from perfusate by centrifugation at 5000×g for 15 minutes at room temperature, which separates cells from, e.g., contaminating debris and platelets. In another embodiment, placental perfusate is concentrated to about 200 ml, gently layered over Ficoll, and centrifuged at about 1100×g for 20 minutes at 22° C., and the low-density interface layer of cells is collected for further processing.

Cell pellets can be resuspended in fresh stem cell collection composition, or a medium suitable for cell maintenance, e.g., stem cell maintenance, for example, IMDM serum-free medium containing 2U/ml heparin and 2 mM EDTA (GibcoBRL, NY). The total mononuclear cell fraction can be isolated, e.g., using Lymphoprep (Nycomed Pharma, Oslo, Norway) according to the manufacturer's recommended procedure.

Placental cells obtained by perfusion or digestion can, for example, be further, or initially, isolated by differential trypsinization using, e.g., a solution of 0.05% trypsin with 0.2% EDTA (Sigma, St. Louis Mo.). Differential trypsinization is possible because the isolated placental cells, which are tissue culture plastic-adherent, typically detach from the plastic surfaces within about five minutes whereas other adherent populations typically require more than 20-30 minutes incubation. The detached placental cells can be harvested following trypsinization and trypsin neutralization, using, e.g., Trypsin Neutralizing Solution (TNS, Cambrex). In one embodiment of isolation of adherent cells, aliquots of, for example, about 5-10×10⁶ cells are placed in each of several T-75 flasks, preferably fibronectin-coated T75 flasks. In such an embodiment, the cells can be cultured with commercially available Mesenchymal Stem Cell Growth Medium (MSCGM) (Cambrex), and placed in a tissue culture incubator (37° C., 5% CO₂). After 10 to 15 days, non-adherent cells are removed from the flasks by washing with PBS. The PBS is then replaced by MSCGM. Flasks are preferably examined daily for the presence of various adherent cell types and in particular, for identification and expansion of clusters of fibroblastoid cells.

The number and type of cells collected from a mammalian placenta can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. For example, using antibodies to CD34, one can determine, using the techniques above, whether a cell comprises a detectable amount of CD34; if so, the cell is CD34⁺. Likewise, if a cell produces enough OCT-4 RNA to be detectable by RT-PCR, or significantly more OCT-4 RNA than an adult cell, the cell is OCT-4⁺. Antibodies to cell surface markers (e.g., CD markers such as CD34) and the sequence of stem cell-specific genes, such as OCT-4, are well-known in the art.

Placental cells, particularly cells that have been isolated by Ficoll separation, differential adherence, or a combination of both, may be sorted using a fluorescence activated cell sorter (FACS). Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one sorting scheme, cells from placenta, e.g., PDACs are sorted on the basis of expression of one or more of the markers CD34, CD38, CD44, CD45, CD73, CD105, OCT-4 and/or HLA-G. This can be accomplished in connection with procedures to select such cells on the basis of their adherence properties in culture. For example, tissue culture plastic adherence selection can be accomplished before or after sorting on the basis of marker expression. In one embodiment, for example, cells are sorted first on the basis of their expression of CD34; CD34⁻ cells are retained, and CD34⁻ cells that are additionally CD200⁺ and HLA-G⁻ are separated from all other CD34⁻ cells. In another embodiment, cells from placenta are sorted based on their expression of markers CD200 and/or HLA-G; for example, cells displaying CD200 and lacking HLA-G are isolated for further use. Cells that express, e.g., CD200 and/or lack, e.g., HLA-G can, in a specific embodiment, be further sorted based on their expression of CD73 and/or CD105, or epitopes recognized by antibodies SH2, SH3 or SH4, or lack of expression of CD34, CD38 or CD45. For example, in another embodiment, placental cells are sorted by expression, or lack thereof, of CD200, HLA-G, CD73, CD105, CD34, CD38 and CD45, and placental cells that are CD200⁺, HLA-G⁻, CD73⁺, CD105⁺, CD34⁻, CD38⁻ and CD45− are isolated from other placental cells for further use.

In specific embodiments of any of the above embodiments of sorted placental cells, at least 50%, 60%, 70%, 80%, 90% or 95% of the cells in a cell population remaining after sorting are said isolated placental cells. Placental cells can be sorted by one or more of any of the markers described in Section 5.2.2, above.

In a specific embodiment, for example, placental cells that are (1) adherent to tissue culture plastic, and (2) CD10⁺, CD34⁻ and CD105⁺ are sorted from (i.e., isolated from) other placental cells. In another specific embodiment, placental cells that are (1) adherent to tissue culture plastic, and (2) CD10⁺, CD34⁻, CD105⁺ and CD200⁺ are sorted from (i.e., isolated from) other placental cells. In another specific embodiment, placental cells that are (1) adherent to tissue culture plastic, and (2) CD10⁺, CD34⁻, CD45⁻, CD90⁺, CD105⁺ and CD200⁺ are sorted from (i.e., isolated from) other placental cells.

With respect to nucleotide sequence-based detection of placental cells, sequences for the markers listed herein are readily available in publicly-available databases such as GenBank or EMBL.

With respect to antibody-mediated detection and sorting of placental cells, e.g., placental stem cells or placental multipotent cells, any antibody, specific for a particular marker, can be used, in combination with any fluorophore or other label suitable for the detection and sorting of cells (e.g., fluorescence-activated cell sorting). Antibody/fluorophore combinations to specific markers include, but are not limited to, fluorescein isothiocyanate (FITC) conjugated monoclonal antibodies against HLA-G (available from Serotec, Raleigh, N.C.), CD10 (available from BD Immunocytometry Systems, San Jose, Calif.), CD44 (available from BD Biosciences Pharmingen, San Jose, Calif.), and CD105 (available from R&D Systems Inc., Minneapolis, Minn.); phycoerythrin (PE) conjugated monoclonal antibodies against CD44, CD200, CD117, and CD13 (BD Biosciences Pharmingen); phycoerythrin-Cy7 (PE Cy7) conjugated monoclonal antibodies against CD33 and CD10 (BD Biosciences Pharmingen); allophycocyanin (APC) conjugated streptavidin and monoclonal antibodies against CD38 (BD Biosciences Pharmingen); and Biotinylated CD90 (BD Biosciences Pharmingen). Other antibodies that can be used include, but are not limited to, CD133-APC (Miltenyi), KDR-Biotin (CD309, Abcam), CytokeratinK-Fitc (Sigma or Dako), HLA ABC-Fitc (BD), HLA DR,DQ,DP-PE (BD), β-2-microglobulin-PE (BD), CD80-PE (BD) and CD86-APC (BD). Other antibody/label combinations that can be used include, but are not limited to, CD45− PerCP (peridin chlorophyll protein); CD44-PE; CD19-PE; CD10-F (fluorescein); HLA-G-F and 7-amino-actinomycin-D (7-AAD); HLA-ABC-F; and the like. This list is not exhaustive, and other antibodies from other suppliers are also commercially available.

The isolated placental cells provided herein can be assayed for CD117 or CD133 using, for example, phycoerythrin-Cy5 (PE Cy5) conjugated streptavidin and biotin conjugated monoclonal antibodies against CD117 or CD133; however, using this system, the cells can appear to be positive for CD117 or CD133, respectively, because of a relatively high background.

The isolated placental cells can be labeled with an antibody to a single marker and detected and/sorted. Placental cells can also be simultaneously labeled with multiple antibodies to different markers.

In another embodiment, magnetic beads can be used to separate cells. The cells may be sorted using a magnetic activated cell sorting (MACS) technique, a method for separating particles based on their ability to bind magnetic beads (0.5-100 m diameter). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

Isolated placental cells can also be characterized and/or sorted based on cell morphology and growth characteristics. For example, isolated placental cells can be characterized as having, and/or selected on the basis of, e.g., a fibroblastoid appearance in culture. The isolated placental cells can also be characterized as having, and/or be selected, on the basis of their ability to form embryoid-like bodies. In one embodiment, for example, placental cells that are fibroblastoid in shape, express CD73 and CD105, and produce one or more embryoid-like bodies in culture are isolated from other placental cells. In another embodiment, OCT-4⁺ placental cells that produce one or more embryoid-like bodies in culture are isolated from other placental cells.

In another embodiment, isolated placental cells can be identified and characterized by a colony forming unit assay. Colony forming unit assays are commonly known in the art, such as MESENCULT™ medium (Stem Cell Technologies, Inc., Vancouver British Columbia).

The isolated placental cells can be assessed for viability, proliferation potential, and longevity using standard techniques known in the art, such as trypan blue exclusion assay, fluorescein diacetate uptake assay, propidium iodide uptake assay (to assess viability); and thymidine uptake assay, MTT (3-(4,5-Dimethylthiazol-2-yl)-2,5-diphenyltetrazolium bromide) cell proliferation assay (to assess proliferation). Longevity may be determined by methods well known in the art, such as by determining the maximum number of population doubling in an extended culture.

Isolated placental cells, e.g., the isolated placental cells described in Section 5.2.2, above, can also be separated from other placental cells using other techniques known in the art, e.g., selective growth of desired cells (positive selection), selective destruction of unwanted cells (negative selection); separation based upon differential cell agglutinability in the mixed population as, for example, with soybean agglutinin; freeze-thaw procedures; filtration; conventional and zonal centrifugation; centrifugal elutriation (counter-streaming centrifugation); unit gravity separation; countercurrent distribution; electrophoresis; and the like.

### 5.3.6 Stimulated Placental Cells

Also provided herein are populations of isolated placental cells, e.g., the isolated placental cells described in Section 5.2.2, above, that have been stimulated by contacting said cells with one or more stimulatory molecules. In a specific embodiment, said cell or population of cells (e.g., PDACs) is stimulated with one or more cytokines. In another specific embodiment, said cell or population of cells (e.g., PDACs) is stimulated with one or more pro-inflammatory cytokines. In a specific embodiment, said one or more pro-inflammatory cytokine is selected from the group consisting of IL-1 α, IL-1 β, IL-6, IL-8, IL-18, TNF-α, and INF-γ. In another specific embodiment, the cell or population of cells described herein (e.g., PDACs) is stimulated with IL-1 α. In another specific embodiment, the cell or population of cells described herein (e.g., PDACs) is stimulated with IL-1 β. In another specific embodiment, the cell or population of cells described herein (e.g., PDACs) is stimulated with IL-6. In another specific embodiment, the cell or population of cells described herein (e.g., PDACs) is stimulated with IL-8. In another specific embodiment, the cell or population of cells described herein (e.g., PDACs) is stimulated with IL-18. In another specific embodiment, the cell or population of cells described herein (e.g., PDACs) is stimulated with TNF-α. In another specific embodiment, the cell or population of cells described herein (e.g., PDACs) is stimulated with INF-γ.

In one embodiment, the population of cells described herein (e.g., PDACs) are stimulated by one or more cytokines, wherein said cytokine is at a concentration of 1 pg/mL, 10 pg/mL, 100 pg/mL, 1,000 pg/mL, 10,000 pg/mL, or 100,000 pg/mL. In a specific embodiment, said culture medium is supplemented with said one or more cytokines at a concentration of 1 pg/mL to 10 pg/mL, 10 pg/mL to 100 pg/mL, 100 pg/mL to 1,000 pg/mL, 1,000 pg/mL to 10,000 pg/mL, or 10,000 pg/mL to 100,000 pg/mL.

In another specific embodiment, the population of cells described herein (e.g., PDACs) are stimulated with one or more cytokines, wherein said stimulation occurs for 1 minute, 5 minutes, 15 minutes, 30 minutes, 60 minutes, 2 hours, 5 hours, 10 hours, 15 hours, 20 hours, 25 hours, 30 hours, 35 hours, 40 hours, 45 hours, 50 hours, 55 hours, or 60 hours. In another specific embodiment, the population of cells described herein (e.g., PDACs) are stimulated with one or more cytokines, wherein said stimulation occurs for between 1 minute and 5 minutes, 5 minutes and 10 minutes, 10 minutes and 15 minutes, 15 minutes and 30 minutes, 30 minutes and 60 minutes, 60 minutes and 2 hours, 2 hours and 5 hours, 5 hours and 10 hours, 10 hours and 15 hours, 15 hours and 20 hours, 20 hours and 25 hours, 25 hours and 30 hours, 30 hours and 35 hours, 35 hours and 40 hours, 40 hours and 45 hours, 45 hours and 50 hours, 50 hours and 55 hours, or 55 hours to 60 hours.

In some embodiments, the stimulated PDACs described herein (e.g., IL-1β-stimulated PDACs) produce secreted factors at a higher level than non-stimulated PDACs. In a specific embodiment, said secreted factors comprise GM-CSF, G-CSF, IL-6, GRO, MCP-1, Follistatin, and/or IL-8. In specific embodiments, said stimulated PDACs described herein (e.g., IL-1β-stimulated PDACs) have pro-angiogenic properties.

In a specific embodiment, the population of cells described herein (e.g., PDACs) are stimulated in vitro.

### 5.4 Culture of Isolated Placental Cells

### 5.4.1 Culture Media

Isolated placental cells, or populations of isolated placental cells, or cells or placental tissue from which placental cells grow out, can be used to initiate, or seed, cell cultures. Cells are generally transferred to sterile tissue culture vessels either uncoated or coated with extracellular matrix or ligands such as laminin, collagen (e.g., native or denatured), gelatin, fibronectin, ornithine, vitronectin, polylysine, CELLSTART™, and/or extracellular membrane protein (e.g., MATRIGEL® (BD Discovery Labware, Bedford, Mass.)), or other suitable biomolecule or synthetic mimetic agent.

Isolated placental cells can be cultured in any medium, and under any conditions, recognized in the art as acceptable for the culture of cells, e.g., stem cells. Preferably, the culture medium comprises serum. The isolated placental cells can be cultured in, for example, DMEM-LG (Dulbecco's Modified Essential Medium, low glucose)/MCDB 201 (chick fibroblast basal medium) containing ITS (insulin-transferrin-selenium), LA+BSA (linoleic acid-bovine serum albumin), dexamethasone L-ascorbic acid, PDGF, EGF, IGF-1, and penicillin/streptomycin; DMEM-HG (high glucose) comprising 10% fetal bovine serum (FBS); DMEM-HG comprising 15% FBS; IMDM (Iscove's modified Dulbecco's medium) comprising 10% FBS, 10% horse serum, and hydrocortisone; M199 comprising 1% to 20% FBS, EGF, and heparin; α-MEM (minimal essential medium) comprising 10% FBS, GLUTAMAX™ and gentamicin; DMEM comprising 10% FBS, GLUTAMAX™ and gentamicin, etc.

Other media in that can be used to culture placental cells include DMEM (high or low glucose), Eagle's basal medium, Ham's F10 medium (F10), Ham's F-12 medium (F12), Iscove's modified Dulbecco's medium, Mesenchymal Stem Cell Growth Medium (MSCGM), Liebovitz's L-15 medium, MCDB, DMEM/F12, RPMI 1640, advanced DMEM (Gibco), DMEM/MCDB201 (Sigma), and CELL-GRO FREE.

The culture medium can be supplemented with one or more components including, for example, serum (e.g., fetal bovine serum (FBS), preferably about 2-15% (v/v); equine (horse) serum (ES); human serum (HS)); beta-mercaptoethanol (BME), preferably about 0.001% (v/v); one or more growth factors, for example, platelet-derived growth factor (PDGF), epidermal growth factor (EGF), basic fibroblast growth factor (bFGF), insulin-like growth factor-1 (IGF-1), leukemia inhibitory factor (LIF), vascular endothelial growth factor (VEGF), and erythropoietin (EPO); amino acids, including L-valine; and one or more antibiotic and/or antimycotic agents to control microbial contamination, such as, for example, penicillin G, streptomycin sulfate, amphotericin B, gentamicin, and nystatin, either alone or in combination.

The isolated placental cells can be cultured in standard tissue culture conditions, e.g., in tissue culture dishes or multiwell plates. The isolated placental cells can also be cultured using a hanging drop method. In this method, isolated placental cells are suspended at about 1×10⁴ cells per mL in about 5 mL of medium, and one or more drops of the medium are placed on the inside of the lid of a tissue culture container, e.g., a 100 mL Petri dish. The drops can be, e.g., single drops, or multiple drops from, e.g., a multichannel pipetter. The lid is carefully inverted and placed on top of the bottom of the dish, which contains a volume of liquid, e.g., sterile PBS sufficient to maintain the moisture content in the dish atmosphere, and the stem cells are cultured.

In specific embodiments, isolated placental stem cells are cultured in the presence of pro-inflammatory cytokines. Culture medium containing pro-inflammatory cytokines can be any medium, and under any conditions, recognized in the art as acceptable for the culture of cells, e.g., stem cells, additionally comprising one or more pro-inflammatory cytokines. In a specific embodiment, said pro-inflammatory cytokines comprise one or more of IL-1 α, IL-1 β, IL-6, IL-8, IL-18, TNF-α, and/or INF-γ. In another specific embodiment, said pro-inflammatory cytokine is IL-1β.

Pro-inflammatory cytokines can be supplemented to culture medium at any concentration recognized in the art as acceptable. In a specific embodiment, said culture medium is supplemented with said one or more cytokines at a concentration of 1 pg/mL, 10 pg/mL, 100 pg/mL, 1,000 pg/mL, 10,000 pg/mL, or 100,000 pg/mL. In a specific embodiment, said culture medium is supplemented with said one or more cytokines at a concentration of 1 pg/mL to 10 pg/mL, 10 pg/mL to 100 pg/mL, 100 pg/mL to 1,000 pg/mL, 1,000 pg/mL to 10,000 pg/mL, or 10,000 pg/mL to 100,000 pg/mL.

In one embodiment, isolated placental cells are cultured in the presence of a compound that acts to maintain an undifferentiated phenotype in the isolated placental cells. In a specific embodiment, the compound is a substituted 3,4-dihydropyridimol[4,5-d]pyrimidine. In another specific embodiment, the compound is a compound having the following chemical structure:

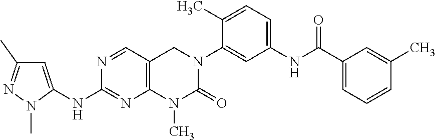

The compound can be contacted with isolated placental cells, or a population of isolated placental cells, at a concentration of, for example, between about 1 M to about 10 M.

### 5.4.2 Expansion and Proliferation of Placental Cells

Once an isolated placental cell, or population of isolated placental cells (e.g., a placental cell or population of placental cells separated from at least 50% of the placental cells with which the stem cell or population of stem cells is normally associated in vivo), the cell or population of cells can be proliferated and expanded in vitro. For example, a population of the isolated placental cells can be cultured in tissue culture containers, e.g., dishes, flasks, multiwell plates, or the like, for a sufficient time for the cells to proliferate to 70-90% confluence, that is, until the cells and their progeny occupy 70-90% of the culturing surface area of the tissue culture container.

The isolated placental cells can be seeded in culture vessels at a density that allows cell growth. For example, the cells may be seeded at low density (e.g., about 1,000 to about 5,000 cells/cm²) to high density (e.g., about 50,000 or more cells/cm²). In a preferred embodiment, the cells are cultured in the presence of about 0 to about 5 percent by volume CO₂ in air. In some preferred embodiments, the cells are cultured at about 2 to about 25 percent O₂ in air, preferably about 5 to about 20 percent O₂ in air. The cells preferably are cultured at about 25° C. to about 40° C., preferably 37° C. The cells are preferably cultured in an incubator. The culture medium can be static or agitated, for example, using a bioreactor. Placental cells, e.g., placental stem cells or placental multipotent cells, preferably are grown under low oxidative stress (e.g., with addition of glutathione, ascorbic acid, catalase, tocopherol, N-acetylcysteine, or the like).

Once confluence of less than 100%, for example, 70% to 90% is obtained, the cells may be passaged. For example, the cells can be enzymatically treated, e.g., trypsinized, using techniques well-known in the art, to separate them from the tissue culture surface. After removing the cells by pipetting and counting the cells, about 10,000-100,000 cells/cm² are passaged to a new culture container containing fresh culture medium. Typically, the new medium is the same type of medium from which the isolated placental cells were removed. The isolated placental cells can be passaged about, at least, or no more than 1, 2, 3, 4, 5, 6, 7, 8, 9, 10, 12, 14, 16, 18, or 20 times, or more.

### 5.4.3 Populations of Isolated Placental Cells

Also provided herein are populations of isolated placental cells, e.g., the isolated placental cells described in Section 5.2.2, above, for example the isolated placental cells used to generate the stimulated PDACs described herein, which are useful in the methods and compositions described herein. Populations of isolated placental cells used to generate the stimulated PDACs described herein can be isolated directly from one or more placentas; that is, the cell population can be a population of placental cells comprising the isolated placental cells, wherein the isolated placental cells are obtained from, or contained within, perfusate, or obtained from, or contained within, disrupted placental tissue, e.g., placental tissue digestate (that is, the collection of cells obtained by enzymatic digestion of a placenta or part thereof). The isolated placental cells used to generate the stimulated PDACs described herein can also be cultured and expanded to produce populations of the isolated placental cells. Populations of placental cells comprising the isolated placental cells used to generate the stimulated PDACs described herein can also be cultured and expanded to produce placental cell populations. In one embodiment, the isolated placental cells described herein are stimulated with one or more pro-inflammatory cytokine. In a specific embodiment, said pro-inflammatory cytokines comprise one or more of IL-1 α, IL-1 β, IL-6, IL-8, IL-18, TNF-α, and INF-γ. In a specific embodiment, said pro-inflammatory cytokine is IL-1β.

Placental cell populations useful in the methods of treatment provided herein comprise the isolated placental cells, for example, the isolated placental cells as described in Section 5.4.2 herein. In a specific embodiment, said placental cell populations comprise PDACs, e.g., IL-1β-stimulated PDACs. In various embodiments, at least 10%, 20%, 30%, 40%, 50%, 60%, 70%, 80%, 90%, 95%, or 99% of the cells in a placental cell population are the isolated placental cells. That is, a population of the isolated placental cells can comprise, e.g., as much as 1%, 5%, 10%, 20%, 30%, 40%, 50%, 60%, 70%, 80%, 90% cells that are not the isolated placental cells.

Isolated placental cell populations useful in the methods and compositions described herein can be produced by, e.g., selecting isolated placental cells, whether derived from enzymatic digestion or perfusion, that express particular markers and/or particular culture or morphological characteristics. In one embodiment, for example, provided herein is a method of producing a cell population by selecting placental cells that (a) adhere to a substrate, and (b) express CD200 and lack expression of HLA-G; and isolating said cells from other cells to form a cell population. In another embodiment, a cell population is produced by selecting placental cells that express CD200 and lack expression of HLA-G, and isolating said cells from other cells to form a cell population. In another embodiment, a cell population is produced by selecting placental cells that (a) adhere to a substrate, and (b) express CD73, CD105, and CD200; and isolating said cells from other cells to form a cell population. In another embodiment, a cell population is produced by identifying placental cells that express CD73, CD105, and CD200, and isolating said cells from other cells to form a cell population. In another embodiment, a cell population is produced by selecting placental cells that (a) adhere to a substrate and (b) express CD200 and OCT-4; and isolating said cells from other cells to form a cell population. In another embodiment, a cell population is produced by selecting placental cells that express CD200 and OCT-4, and isolating said cells from other cells to form a cell population. In another embodiment, a cell population is produced by selecting placental cells that (a) adhere to a substrate, (b) express CD73 and CD105, and (c) facilitate the formation of one or more embryoid-like bodies in a population of placental cells comprising said stem cell when said population is cultured under conditions that allow for the formation of an embryoid-like body; and isolating said cells from other cells to form a cell population. In another embodiment, a cell population is produced by selecting placental cells that express CD73 and CD105, and facilitate the formation of one or more embryoid-like bodies in a population of placental cells comprising said stem cell when said population is cultured under conditions that allow for the formation of an embryoid-like body, and isolating said cells from other cells to form a cell population. In another embodiment, a cell population is produced by selecting placental cells that (a) adhere to a substrate, and (b) express CD73 and CD105, and lack expression of HLA-G; and isolating said cells from other cells to form a cell population. In another embodiment, a cell population is produced by selecting placental cells that express CD73 and CD105 and lack expression of HLA-G, and isolating said cells from other cells to form a cell population. In another embodiment, the method of producing a cell population comprises selecting placental cells that (a) adhere to a substrate, (b) express OCT-4, and (c) facilitate the formation of one or more embryoid-like bodies in a population of placental cells comprising said stem cell when said population is cultured under conditions that allow for the formation of an embryoid-like body; and isolating said cells from other cells to form a cell population. In another embodiment, a cell population is produced by selecting placental cells that express OCT-4, and facilitate the formation of one or more embryoid-like bodies in a population of placental cells comprising said stem cell when said population is cultured under conditions that allow for the formation of an embryoid-like body, and isolating said cells from other cells to form a cell population.

In another embodiment, a cell population, e.g., a cell population used to generate the stimulated PDACs described herein, is produced by selecting placental cells that (a) adhere to a substrate, and (b) express CD10 and CD105, and do not express CD34; and isolating said cells from other cells to form a cell population. In another embodiment, a cell population is produced by selecting placental cells that express CD10 and CD105, and do not express CD34, and isolating said cells from other cells to form a cell population. In another embodiment, a cell population, e.g., a cell population used to generate the stimulated PDACs described herein, is produced by selecting placental cells that (a) adhere to a substrate, and (b) express CD10, CD105, and CD200, and do not express CD34; and isolating said cells from other cells to form a cell population. In another embodiment, a cell population is produced by selecting placental cells that express CD10, CD105, and CD200, and do not express CD34, and isolating said cells from other cells to form a cell population. In another specific embodiment, a cell population, e.g., a cell population used to generate the stimulated PDACs described herein, is produced by selecting placental cells that (a) adhere to a substrate, and (b) express CD10, CD90, CD105 and CD200, and do not express CD34 and CD45; and isolating said cells from other cells to form a cell population. In another specific embodiment, a cell population, e.g., a cell population used to generate the stimulated PDACs described herein, is produced by selecting placental cells that express CD10, CD90, CD105 and CD200, and do not express CD34 and CD45, and isolating said cells from other cells to form a cell population.

Selection of cell populations comprising placental cells having any of the marker combinations described in Section 5.2.2, above, can be isolated or obtained in similar fashion.

In any of the above embodiments, selection of the isolated cell populations can additionally comprise selecting placental cells that express ABC-p (a placenta-specific ABC transporter protein; see, e.g., Allikmets et al., Cancer Res. 58(23):5337-9 (1998)). The method can also comprise selecting cells exhibiting at least one characteristic specific to, e.g., a mesenchymal stem cell, for example, expression of CD44, expression of CD90, or expression of a combination of the foregoing.

In the above embodiments, the substrate can be any surface on which culture and/or selection of cells, e.g., isolated placental cells, can be accomplished. Typically, the substrate is plastic, e.g., tissue culture dish or multiwell plate plastic. Tissue culture plastic can be coated with a biomolecule, e.g., laminin or fibronectin.

Cells, e.g., the isolated placental cells used to generate the stimulated PDACs described herein, can be selected for a placental cell population by any means known in the art of cell selection. For example, cells can be selected using an antibody or antibodies to one or more cell surface markers, for example, in flow cytometry or FACS. Selection can be accomplished using antibodies in conjunction with magnetic beads. Antibodies that are specific for certain stem cell-related markers are known in the art. For example, antibodies to OCT-4 (Abcam, Cambridge, Mass.), CD200 (Abcam), HLA-G (Abcam), CD73 (BD Biosciences Pharmingen, San Diego, Calif.), CD105 (Abcam; BioDesign International, Saco, Me.), etc. Antibodies to other markers are also available commercially, e.g., CD34, CD38 and CD45 are available from, e.g., StemCell Technologies or BioDesign International.

The isolated placental cell populations used to generate the stimulated PDACs described herein can comprise placental cells that are not stem cells, or cells that are not placental cells.

The isolated cell populations comprising placental derived adherent cells used to generate the stimulated PDACs described herein can comprise a second cell type, e.g., placental cells that are not placental derived adherent cells, or, e.g., cells that are not placental cells. For example, an isolated population of placental derived adherent cells can comprise, e.g., can be combined with, a population of a second type of cells, wherein said second type of cell are, e.g., embryonic stem cells, blood cells (e.g., placental blood, placental blood cells, umbilical cord blood, umbilical cord blood cells, peripheral blood, peripheral blood cells, nucleated cells from placental blood, umbilical cord blood, or peripheral blood, and the like), stem cells isolated from blood (e.g., stem cells isolated from placental blood, umbilical cord blood or peripheral blood), nucleated cells from placental perfusate, e.g., total nucleated cells from placental perfusate; umbilical cord stem cells, populations of blood-derived nucleated cells, bone marrow-derived mesenchymal stromal cells, bone marrow-derived mesenchymal stem cells, bone marrow-derived hematopoietic stem cells, crude bone marrow, adult (somatic) stem cells, populations of stem cells contained within tissue, cultured cells, e.g., cultured stem cells, populations of fully-differentiated cells (e.g., chondrocytes, fibroblasts, amniotic cells, osteoblasts, muscle cells, cardiac cells, etc.), pericytes, and the like. In a specific embodiment, a population of cells comprising placental derived adherent cells comprises placental stem cells or stem cells from umbilical cord. In certain embodiments in which the second type of cell is blood or blood cells, erythrocytes have been removed from the population of cells.

In a specific embodiment, the second type of cell is a hematopoietic stem cell. Such hematopoietic stem cells can be, for example, contained within unprocessed placental, umbilical cord blood or peripheral blood; in total nucleated cells from placental blood, umbilical cord blood or peripheral blood; in an isolated population of CD34⁺ cells from placental blood, umbilical cord blood or peripheral blood; in unprocessed bone marrow; in total nucleated cells from bone marrow; in an isolated population of CD34⁺ cells from bone marrow, or the like.

In another embodiment, an isolated population of placental derived adherent cells used to generate the stimulated PDACs described herein is combined with a plurality of adult or progenitor cells from the vascular system. In various embodiments, the cells are endothelial cells, endothelial progenitor cells, myocytes, cardiomyocytes, pericytes, angioblasts, myoblasts or cardiomyoblasts.

In a another embodiment, the second cell type is a non-embryonic cell type manipulated in culture in order to express markers of pluripotency and functions associated with embryonic stem cells

In specific embodiments of the above isolated populations of placental derived adherent cells used to generate the stimulated PDACs described herein, either or both of the placental derived adherent cells and cells of a second type are autologous, or are allogeneic, to an intended recipient of the cells.

In another specific embodiment, the composition comprises placental derived adherent cells, and embryonic stem cells. In another specific embodiment, the composition comprises placental derived adherent cells and mesenchymal stromal or stem cells, e.g., bone marrow-derived mesenchymal stromal or stem cells. In another specific embodiment, the composition comprises bone marrow-derived hematopoietic stem cells. In another specific embodiment, the composition comprises placental derived adherent cells and hematopoietic progenitor cells, e.g., hematopoietic progenitor cells from bone marrow, fetal blood, umbilical cord blood, placental blood, and/or peripheral blood. In another specific embodiment, the composition comprises placental derived adherent cells and somatic stem cells. In a more specific embodiment, said somatic stem cell is a neural stem cell, a hepatic stem cell, a pancreatic stem cell, an endothelial stem cell, a cardiac stem cell, or a muscle stem cell.

In other specific embodiments, the second type of cells comprise about, at least, or no more than, 10%, 15%, 20%, 25%, 30%, 35%, 40%, 45%, or 50% of cells in said population. In other specific embodiments, the PDAC in said composition comprise at least 50%, 55%, 60%, 65%, 70%, 75%, 80%, 85% or 90% of cells in said composition. In other specific embodiments, the placental derived adherent cells comprise about, at least, or no more than, 10%, 15%, 20%, 25%, 30%, 35%, 40%, or 45% of cells in said population.

Cells in an isolated population of placental derived adherent cells can be combined with a plurality of cells of another type, e.g., with a population of stem cells, in a ratio of about 100,000,000:1, 50,000,000:1, 20,000,000:1, 10,000,000:1, 5,000,000:1, 2,000,000:1, 1,000,000:1, 500,000:1, 200,000:1, 100,000:1, 50,000:1, 20,000:1, 10,000:1, 5,000:1, 2,000:1, 1,000:1, 500:1, 200:1, 100:1, 50:1, 20:1, 10:1, 5:1, 2:1, 1:1; 1:2; 1:5; 1:10; 1:100; 1:200; 1:500; 1:1,000; 1:2,000; 1:5,000; 1:10,000; 1:20,000; 1:50,000; 1:100,000; 1:500,000; 1:1,000,000; 1:2,000,000; 1:5,000,000; 1:10,000,000; 1:20,000,000; 1:50,000,000; or about 1:100,000,000, comparing numbers of total nucleated cells in each population. Cells in an isolated population of placental derived adherent cells can be combined with a plurality of cells of a plurality of cell types, as well.

In other embodiments, a population of the placental cells described herein, e.g., the PDACs described above, are combined with osteogenic placental adherent cells (OPACs), e.g., the OPACs described in patent application Ser. No. 12/546,556, filed Aug. 24, 2009, entitled “Methods and Compositions for Treatment of Bone Defects With Placental Stem Cells,” or combined with amnion-derived angiogenic cells (AMDACs), e.g., the AMDACs described in U.S. patent application Ser. No. 12/622,352, entitled “Amnion Derived Angiogenic Cells”, the disclosure of which is hereby incorporated by reference in its entirety.

In one embodiment, the isolated placental cells described herein (e.g., PDACs) are stimulated with one or more pro-inflammatory cytokines. In a specific embodiment, said pro-inflammatory cytokines comprise one or more of IL-1 α, IL-1 β, IL-6, IL-8, IL-18, TNF-α, and INF-γ. In a specific embodiment, said pro-inflammatory cytokine is IL-10.

### 5.5 Production of a Placental Cell Bank

Isolated cells from postpartum placentas, e.g., the isolated placental cells described in Section 5.2.2, above, can be cultured in a number of different ways to produce a set of lots, e.g., wherein a lot is a set of individually-administrable doses, of isolated placental cells. Such lots can, for example, be obtained from cells from placental perfusate or from cells from enzyme-digested placental tissue. Sets of lots of placental cells, obtained from a plurality of placentas, can be arranged in a bank of isolated placental cells for, e.g., long-term storage. Generally, tissue culture plastic-adherent placental cells are obtained from an initial culture of placental material to form a seed culture, which is expanded under controlled conditions to form populations of cells from approximately equivalent numbers of doublings. Lots are preferably derived from the tissue of a single placenta, but can be derived from the tissue of a plurality of placentas.

In one embodiment, placental cell lots are obtained as follows. Placental tissue is first disrupted, e.g., by mincing, digested with a suitable enzyme, e.g., trypsin or collagenase (see Section 5.3.3, above). The placental tissue preferably comprises, e.g., the entire amnion, entire chorion, or both, from a single placenta, but can comprise only a part of either the amnion or chorion. The digested tissue is cultured, e.g., for about 1-3 weeks, preferably about 2 weeks. After removal of non-adherent cells, high-density colonies that form are collected, e.g., by trypsinization. These cells are collected and resuspended in a convenient volume of culture medium, and are then used to seed expansion cultures. Expansion cultures can be any arrangement of separate cell culture apparatuses, e.g., a Cell Factory by NUNC™. Cells can be subdivided to any degree so as to seed expansion cultures with, e.g., 1×10³, 2×10³, 3×10³, 4×10³ 5×10³ 6×10³ 7×10³ 8×10³ 9×10³ 1×10⁴ 1×10⁴ 2×10⁴ 3×10⁴ 4×10⁴ 5×10⁴ 6×10⁴, 7×10⁴, 8×10⁴, 9×10⁴, or 10×10⁴ cells/cm². Preferably, from about 1×10³ to about 1×10⁴ cells/cm² are used to seed each expansion culture. The number of expansion cultures may be greater or fewer in number depending upon the particular placenta(s) from which the cells are obtained.

Expansion cultures are grown until the density of cells in culture reaches a certain value, e.g., about 1×10⁵ cells/cm². Cells can either be collected and cryopreserved at this point, or passaged into new expansion cultures as described above. Cells can be passaged, e.g., 2, 3, 4, 5, 6, 7, 8, 9, 10, 11, 12, 13, 14, 15, 16, 17, 18, 19 or 20 times prior to use. A record of the cumulative number of population doublings is preferably maintained during expansion culture(s). The cells from a culture can be expanded for 2, 3, 4, 5, 6, 7, 8, 9, 10, 12, 14, 16, 18, 20, 22, 24, 26, 28, 30, 32, 34, 36, 38 or 40 doublings, or up to 60 doublings. Preferably, however, the number of population doublings, prior to dividing the population of cells into individual doses, is from about 15 to about 30. The cells can be culture continuously throughout the expansion process, or can be frozen at one or more points during expansion.

Cells to be used for individual doses can be frozen, e.g., cryopreserved for later use. Individual doses can comprise, e.g., about 1 million to about 50 million cells per ml, and can comprise between about 10⁶ and about 10¹⁰ cells in total.

In one embodiment, therefore, a placental cell bank can be made by a method comprising: expanding primary culture placental cells from a human post-partum placenta for a first plurality of population doublings; cryopreserving said placental cells to form a Master Cell Bank; expanding a plurality of placental cells from the Master Cell Bank for a second plurality of population doublings; cryopreserving said placental cells to form a Working Cell Bank; expanding a plurality of placental cells from the Working Cell Bank for a third plurality of population doublings; and cryopreserving said placental cells in individual doses, wherein said individual doses collectively compose a placental cell bank. Optionally, a plurality of placental cells from said third plurality of population doublings can be expanded for a fourth plurality of population doublings and cryopreserved in individual doses, wherein said individual doses collectively compose a placental cell bank.

In another specific embodiment, said primary culture placental cells comprise placental cells from placental perfusate. In another specific embodiment, said primary culture placental cells comprise placental cells from digested placental tissue. In another specific embodiment, said primary culture placental cells comprise placental cells from placental perfusate and from digested placental tissue. In another specific embodiment, all of said placental cells in said placental cell primary culture are from the same placenta. In another specific embodiment, the method further comprises the step of selecting CD200⁺ or HLA-G⁻ placental cells from said plurality of said placental cells from said Working Cell Bank to form individual doses. In another specific embodiment, said individual doses comprise from about 10⁴ to about 10⁵ placental cells. In another specific embodiment, said individual doses comprise from about 10⁵ to about 10⁶ placental cells. In another specific embodiment, said individual doses comprise from about 10⁶ to about 10⁷ placental cells. In another specific embodiment, said individual doses comprise from about 10⁷ to about 10⁸ placental cells. In another specific embodiment, said individual doses comprise from about 10⁸ to about 10⁹ placental cells. In another specific embodiment, said individual doses comprise from about 10⁹ to about 10¹⁰ placental cells.

In a preferred embodiment, the donor from which the placenta is obtained (e.g., the mother) is tested for at least one pathogen. If the mother tests positive for a tested pathogen, the entire lot from the placenta is discarded. Such testing can be performed at any time during production of placental cell lots, e.g., during expansion culture. Pathogens for which the presence is tested can include, without limitation, hepatitis A, hepatitis B, hepatitis C, hepatitis D, hepatitis E, human immunodeficiency virus (types I and II), cytomegalovirus, herpesvirus, and the like.

In one embodiment, the placental stem cells banked according to the methods described herein are stimulated with one or more pro-inflammatory cytokines prior to banking. In a specific embodiment, said pro-inflammatory cytokines comprise one or more of IL-1 α, IL-1 β, IL-6, IL-8, IL-18, TNF-α, and INF-γ. In a specific embodiment, said pro-inflammatory cytokine is IL-1β.

### 5.6 Preservation of Placental Cells

Isolated placental cells, e.g., the isolated placental cells described in Section 5.3.2, above, can be preserved, that is, placed under conditions that allow for long-term storage, or conditions that inhibit cell death by, e.g., apoptosis or necrosis.

Placental cells can be preserved using, e.g., a composition comprising an apoptosis inhibitor, necrosis inhibitor and/or an oxygen-carrying perfluorocarbon, as described in related U.S. Application Publication No. 2007/0190042, the disclosure of which is incorporated herein by reference in its entirety. In one embodiment, a method of preserving a population of cells, useful in the methods and compositions described herein, comprises contacting said population of cells with a cell collection composition comprising an inhibitor of apoptosis and an oxygen-carrying perfluorocarbon, wherein said inhibitor of apoptosis is present in an amount and for a time sufficient to reduce or prevent apoptosis in the population of cells, as compared to a population of cells not contacted with the inhibitor of apoptosis. In a specific embodiment, said inhibitor of apoptosis is a caspase inhibitor. In another specific embodiment, said inhibitor of apoptosis is a INK inhibitor. In another specific embodiment, said INK inhibitor does not modulate differentiation or proliferation of said cells. In another embodiment, said cell collection composition comprises said inhibitor of apoptosis and said oxygen-carrying perfluorocarbon in separate phases. In another embodiment, said cell collection composition comprises said inhibitor of apoptosis and said oxygen-carrying perfluorocarbon in an emulsion. In another embodiment, the cell collection composition additionally comprises an emulsifier, e.g., lecithin. In another embodiment, said apoptosis inhibitor and said perfluorocarbon are between about 0° C. and about 25° C. at the time of contacting the cells. In another specific embodiment, said apoptosis inhibitor and said perfluorocarbon are between about 2° C. and 10° C., or between about 2° C. and about 5° C., at the time of contacting the cells. In another specific embodiment, said contacting is performed during transport of said population of cells. In another specific embodiment, said contacting is performed during freezing and thawing of said population of cells.

Populations of placental cells can be preserved, e.g., by a method comprising contacting said population of cells with an inhibitor of apoptosis and an organ-preserving compound, wherein said inhibitor of apoptosis is present in an amount and for a time sufficient to reduce or prevent apoptosis in the population of cells, as compared to a population of cells not contacted with the inhibitor of apoptosis. In a specific embodiment, the organ-preserving compound is UW solution (described in U.S. Pat. No. 4,798,824; also known as ViaSpan; see also Southard et al., Transplantation 49(2):251-257 (1990)) or a solution described in Stern et al., U.S. Pat. No. 5,552,267, the disclosures of which are hereby incorporated by reference in their entireties. In another embodiment, said organ-preserving compound is hydroxyethyl starch, lactobionic acid, raffinose, or a combination thereof. In another embodiment, the cell collection composition additionally comprises an oxygen-carrying perfluorocarbon, either in two phases or as an emulsion.

In another embodiment of the method, placental cells are contacted with a cell collection composition comprising an apoptosis inhibitor and oxygen-carrying perfluorocarbon, organ-preserving compound, or combination thereof, during perfusion. In another embodiment, said cells are contacted during a process of tissue disruption, e.g., enzymatic digestion. In another embodiment, placental cells are contacted with said cell collection compound after collection by perfusion, or after collection by tissue disruption, e.g., enzymatic digestion.

Typically, during placental cell collection, enrichment and isolation, it is preferable to minimize or eliminate cell stress due to hypoxia and mechanical stress. In another embodiment of the method, therefore, a cell, or population of cells, is exposed to a hypoxic condition during collection, enrichment or isolation for less than six hours during said preservation, wherein a hypoxic condition is a concentration of oxygen that is less than normal blood oxygen concentration. In another specific embodiment, said population of cells is exposed to said hypoxic condition for less than two hours during said preservation. In another specific embodiment, said population of cells is exposed to said hypoxic condition for less than one hour, or less than thirty minutes, or is not exposed to a hypoxic condition, during collection, enrichment or isolation. In another specific embodiment, said population of cells is not exposed to shear stress during collection, enrichment or isolation.

Placental cells can be cryopreserved, e.g., in cryopreservation medium in small containers, e.g., ampoules. Suitable cryopreservation medium includes, but is not limited to, culture medium including, e.g., growth medium, or cell freezing medium, for example commercially available cell freezing medium, e.g., C2695, C2639 or C6039 (Sigma). Cryopreservation medium preferably comprises DMSO (dimethylsulfoxide), at a concentration of about 2% to about 15% (v/v), e.g., about 10% (v/v). Cryopreservation medium may comprise additional agents, for example, methylcellulose and/or glycerol. Placental cells are preferably cooled at about 1° C./min during cryopreservation. A preferred cryopreservation temperature is about −80° C. to about −180° C., preferably about −125° C. to about −140° C. Cryopreserved cells can be transferred to liquid nitrogen prior to thawing for use. In some embodiments, for example, once the ampoules have reached about −90° C., they are transferred to a liquid nitrogen storage area. Cryopreservation can also be done using a controlled-rate freezer. Cryopreserved cells preferably are thawed at a temperature of about 25° C. to about 40° C., preferably to a temperature of about 37° C.

In one embodiment, the placental stem cells cryopreserved according to the methods described herein are stimulated with one or more pro-inflammatory cytokines prior to cryopreservation. In specific embodiments, the pro-inflammatory cytokines comprise one or more of IL-1 α, IL-1 β, IL-6, IL-8, IL-18, TNF-α, and INF-γ. In a specific embodiment, said pro-inflammatory cytokine is IL-1β.

### 5.7 Compositions Comprising Isolated Placental Cells

The stimulated placental cells described herein, e.g., at Section 5.3.6, can be combined with any physiologically-acceptable or medically-acceptable compound, composition or device for use in the methods and compositions described herein. Compositions useful in the methods of treatment provided herein can comprise any one or more of the stimulated placental cells described herein (see Section 5.3.6, above). In certain embodiments, the composition is a pharmaceutically-acceptable composition, e.g., a composition comprising stimulated placental cells in a pharmaceutically-acceptable carrier.

In certain embodiments, a composition comprising the stimulated isolated placental cells additionally comprises a matrix, e.g., a decellularized matrix or a synthetic matrix. In another specific embodiment, said matrix is a three-dimensional scaffold. In another specific embodiment, said matrix comprises collagen, gelatin, laminin, fibronectin, pectin, ornithine, or vitronectin. In another ore specific embodiment, the matrix is an amniotic membrane or an amniotic membrane-derived biomaterial. In another specific embodiment, said matrix comprises an extracellular membrane protein. In another specific embodiment, said matrix comprises a synthetic compound. In another specific embodiment, said matrix comprises a bioactive compound. In another specific embodiment, said bioactive compound is a growth factor, cytokine, antibody, or organic molecule of less than 5,000 daltons.

In another embodiment, a composition useful in the methods of treatment provided herein comprises medium conditioned by any of the foregoing placental cells, or any of the foregoing placental cell populations.

### 5.7.1 Cryopreserved Isolated Placental Cells

The isolated placental cell populations useful in the methods and compositions described herein can be preserved, for example, cryopreserved for later use. Methods for cryopreservation of cells, such as stem cells, are well known in the art. Isolated placental cell populations can be prepared in a form that is easily administrable to an individual, e.g., an isolated placental cell population that is contained within a container that is suitable for medical use. Such a container can be, for example, a syringe, sterile plastic bag, flask, jar, or other container from which the isolated placental cell population can be easily dispensed. For example, the container can be a blood bag or other plastic, medically-acceptable bag suitable for the intravenous administration of a liquid to a recipient. The container, in certain embodiments, is one that allows for cryopreservation of the combined cell population.

In one embodiment, the placental stem cells cryopreserved according to the methods described herein are stimulated with one or more pro-inflammatory cytokines prior to cryopreservation. In another embodiment, said placental stem cells are stimulated with one or more pro-inflammatory cytokines after thawing said cryopreserved placental stem cells. In specific embodiments, the pro-inflammatory cytokines comprise one or more of IL-1 α, IL-1 β, IL-6, IL-8, IL-18, TNF-α, and INF-γ. In a specific embodiment, said pro-inflammatory cytokine is IL-1β.

The cryopreserved isolated placental cell population can comprise isolated placental cell derived from a single donor, or from multiple donors. The isolated placental cell population can be completely HLA-matched to an intended recipient, or partially or completely HLA-mismatched.

Thus, in one embodiment, isolated placental cells can be used in the methods and described herein in the form of a composition comprising a tissue culture plastic-adherent placental cell population in a container. In a specific embodiment, the isolated placental cells are cryopreserved. In another specific embodiment, the container is a bag, flask, or jar. In another specific embodiment, said bag is a sterile plastic bag. In another specific embodiment, said bag is suitable for, allows or facilitates intravenous administration of said isolated placental cell population, e.g., by intravenous infusion. The bag can comprise multiple lumens or compartments that are interconnected to allow mixing of the isolated placental cells and one or more other solutions, e.g., a drug, prior to, or during, administration. In another specific embodiment, the composition comprises one or more compounds that facilitate cryopreservation of the combined cell population. In another specific embodiment, said isolated placental cell population is contained within a physiologically-acceptable aqueous solution. In another specific embodiment, said physiologically-acceptable aqueous solution is a 0.9% NaCl solution. In another specific embodiment, said isolated placental cell population comprises placental cells that are HLA-matched to a recipient of said cell population. In another specific embodiment, said combined cell population comprises placental cells that are at least partially HLA-mismatched to a recipient of said cell population. In another specific embodiment, said isolated placental cells are derived from a plurality of donors.

In certain embodiments, the isolated placental cells in the container are isolated CD10⁺, CD34⁻, CD105⁺ placental cells, wherein said cells have been cryopreserved, and are contained within a container. In a specific embodiment, said CD10⁺, CD34⁻, CD105⁺ placental cells are also CD200⁺. In another specific embodiment, said CD10⁺, CD34⁻, CD105⁺, CD200⁺ placental cells are also CD45⁻ or CD90⁺. In another specific embodiment, said CD10⁺, CD34⁻, CD105⁺, CD200⁺ placental cells are also CD45⁻ and CD90⁺. In another specific embodiment, the CD34⁻, CD10⁺, CD105⁺ placental cells are additionally one or more of CD13⁺, CD29⁺, CD33⁺, CD38⁻, CD44⁺, CD45⁻, CD54⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105), CD106/VCAM⁺, CD117⁻, CD144/VE-cadherin^{dim}, CD184/CXCR4⁻, CD200⁺, CD133⁻, OCT-4⁺, SSEA3⁻, SSEA4⁻, ABC-p⁺, KDR⁻ (VEGFR2−), HLA-A,B,C⁺, HLA-DP,DQ,DR⁻, HLA-G⁻, or Programmed Death-1 Ligand (PDL1)⁺, or any combination thereof. In another specific embodiment, the CD34⁻, CD10⁺, CD105⁺ placental cells are additionally CD13⁺, CD29⁺, CD33⁺, CD38⁻, CD44⁺, CD45⁻, CD54/ICAM⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105), CD106/VCAM⁺, CD117, CD144/VE-cadherin^{dim}, CD184/CXCR4⁻, CD200⁺, CD133⁻, OCT-4⁺, SSEA3⁻, SSEA4⁻, ABC-p⁺, KDR⁻ (VEGFR2−), HLA-A,B,C⁺, HLA-DP,DQ,DR⁻, HLA-G⁻, and Programmed Death-1 Ligand (PDL1)⁺.

In certain other embodiments, the above-referenced isolated placental cells are isolated CD200⁺, HLA-G⁻ placental cells, wherein said cells have been cryopreserved, and are contained within a container. In another embodiment, the isolated placental cells are CD73⁺, CD105⁺, CD200⁺ cells that have been cryopreserved, and are contained within a container. In another embodiment, the isolated placental cells are CD200⁺, OCT-4⁺ stem cells that have been cryopreserved, and are contained within a container. In another embodiment, the isolated placental cells are CD73⁺, CD105⁺ cells that have been cryopreserved, and are contained within a container, and wherein said isolated placental cells facilitate the formation of one or more embryoid-like bodies when cultured with a population of placental cells under conditions that allow for the formation of embryoid-like bodies. In another embodiment, the isolated placental cells are CD73⁺, CD105⁺, HLA-G⁻ cells that have been cryopreserved, and are contained within a container. In another embodiment, the isolated placental cells are OCT-4⁺ placental cells that have been cryopreserved, and are contained within a container, and wherein said cells facilitate the formation of one or more embryoid-like bodies when cultured with a population of placental cells under conditions that allow for the formation of embryoid-like bodies.

In another specific embodiment, the above-referenced isolated placental cells are placental stem cells or placental multipotent cells that are CD34⁻, CD10⁺ and CD105⁺ as detected by flow cytometry (e.g., PDACs). In another specific embodiment, the isolated CD34⁻, CD10⁺, CD105⁺ placental cells have the potential to differentiate into cells of a neural phenotype, cells of an osteogenic phenotype, or cells of a chondrogenic phenotype. In another specific embodiment, the isolated CD34⁻, CD10⁺, CD105⁺ placental cells are additionally CD200⁺. In another specific embodiment, the isolated CD34⁻, CD10⁺, CD105⁺ placental cells are additionally CD90⁺ or CD45⁻, as detected by flow cytometry. In another specific embodiment, the isolated CD34⁻, CD10⁺, CD105⁺ placental cells are additionally CD90⁺ or CD45⁻, as detected by flow cytometry. In another specific embodiment, the CD34⁻, CD10⁺, CD105⁺, CD200⁺ placental cells are additionally CD90⁺ or CD45⁻, as detected by flow cytometry. In another specific embodiment, the CD34⁻, CD10⁺, CD105⁺, CD200⁺ cells are additionally CD90⁺ and CD45⁻, as detected by flow cytometry. In another specific embodiment, the CD34⁻, CD10⁺, CD105⁺, CD200⁺, CD90⁺, CD45⁻ cells are additionally CD80- and CD86⁻, as detected by flow cytometry. In another specific embodiment, the CD34⁻, CD10⁺, CD105⁺ cells are additionally one or more of CD29⁺, CD38⁻, CD44⁺, CD54⁺, CD80⁻, CD86⁻, SH3⁺ or SH4⁺. In another specific embodiment, the cells are additionally CD44⁺. In a specific embodiment of any of the isolated CD34⁻, CD10⁺, CD105⁺ placental cells above, the cells are additionally one or more of CD117⁻, CD133⁻, KDR⁻ (VEGFR2⁻), HLA-A,B,C⁺, HLA-DP,DQ,DR⁻, and/or PDL1⁺.

In a specific embodiment of any of the foregoing cryopreserved isolated placental cells, said container is a bag. In various specific embodiments, said container comprises about, at least, or at most 1×10⁶ said isolated placental cells, 5×10⁶ said isolated placental cells, 1×10⁷ said isolated placental cells, 5×10⁷ said isolated placental cells, 1×10⁸ said isolated placental cells, 5×10⁸ said isolated placental cells, 1×10⁹ said isolated placental cells, 5×10⁹ said isolated placental cells, 1×10¹⁰ said isolated placental cells, or 1×10¹⁰ said isolated placental cells. In other specific embodiments of any of the foregoing cryopreserved populations, said isolated placental cells have been passaged about, at least, or no more than 5 times, no more than 10 times, no more than 15 times, or no more than 20 times. In another specific embodiment of any of the foregoing cryopreserved isolated placental cells, said isolated placental cells have been expanded within said container.

In certain embodiments, a single unit dose of placental derived adherent cells can comprise, in various embodiments, about, at least, or no more than 1×10⁵, 5×10⁵, 1×10⁶, 5×10⁶, 1×10⁷, 5×10⁷, 1×10⁸, 5×10⁸, 1×10⁹, 5×10⁹, 1×10¹°, 5×10¹°, 1×10¹¹ or more placental derived adherent cells. In certain embodiments, the pharmaceutical compositions provided herein comprises populations of placental derived adherent cells, that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

### 5.7.2 Genetically Engineered Placental Cells

Further provided herein are placental cells, e.g., any of the placental multipotent cells or placental cells described in Sections 5.2.2 and 5.3.6, above, or pharmaceutical compositions comprising such placental cells, wherein the placental cells have been genetically engineered to produce recombinant or exogenous cytokines associated with, or which promote, angiogenesis. In certain embodiments, said proteins that facilitate angiogenesis are one or more of hepatocyte growth factor (HGF), vascular endothelial growth factor (VEGF) (e.g., VEGFD), fibroblast growth factor (FGF) (e.g., FGF2), angiogenin (ANG), epidermal growth factor (EGF), epithelial-neutrophil-activating protein 78 (ENA-78), follistatin, granulocyte colony-stimulating factor (G-CSF), growth-regulated oncogene protein (GRO), interleukin-6 (IL-6), IL-8, leptin, monocyte chemotactic protein-1 (MCP-1), MCP-3, platelet-derived growth factor subunit B (PDGFB), rantes, transforming growth factor beta 1 (TGF-β1), thrombopoitein (Tpo), tissue inhibitor of metalloproteinases 1 (TIMP1), TIMP2, and/or urokinase plasminogen activator receptor (uPAR).

Methods for genetically engineering cells, for example with retroviral vectors, adenoviral vectors, adeno-associated viral vectors, polyethylene glycol, or other methods known to those skilled in the art, can be used. These include using expression vectors which transport and express nucleic acid molecules in the cells. (See Geoddel; Gene Expression Technology: Methods in Enzymology 185, Academic Press, San Diego, Calif. (1990)). Vector DNA can be introduced into prokaryotic or eukaryotic cells via conventional transformation or transfection techniques. Suitable methods for transforming or transfecting host cells can be found in Sambrook et al. Molecular Cloning: A Laboratory Manual, 2nd Edition, Cold Spring Harbor Laboratory press (1989), and other laboratory textbooks.

Placental cells, e.g., the PDACs described in Section 5.2, above, can be genetically modified by introducing DNA or RNA into the cell, e.g., DNA or RNA encoding a protein of interest, by methods including viral transfer, including the use of DNA or RNA viral vectors, such as retroviruses (including lentiviruses), Simian virus 40 (SV40), adenovirus, Sindbis virus, and bovine papillomavirus for example; chemical transfer, including calcium phosphate transfection and DEAE dextran transfection methods; membrane fusion transfer, using DNA-loaded membrane vesicles such as liposomes, red blood cell ghosts, and protoplasts, for example; or physical transfer techniques, such as microinjection, electroporation, or naked DNA transfer. The placental cells can be genetically altered by insertion of exogenous DNA, or by substitution of a segment of the cellular genome with exogenous DNA. Insertion of exogenous DNA sequence(s) can be accomplished, e.g., by homologous recombination or by viral integration into the host cell genome, or by incorporating the DNA into the cell, particularly into its nucleus, using a plasmid expression vector and a nuclear localization sequence. The DNA can comprise one or more promoters that allow positive or negative induction of expression of the protein of interest using certain chemicals/drugs, e.g., tetracycline; the promoters can, in other embodiments, be constitutive.

Calcium phosphate transfection can be used to introduce, e.g., plasmid DNA containing a polynucleotide sequence encoding the protein of interest, into a cell. In certain embodiments, DNA is combined with a solution of calcium chloride, then added to a phosphate-buffered solution. Once a precipitate has formed, the solution is added directly to cultured cells. Treatment with DMSO or glycerol can be used to improve transfection efficiency, and levels of stable transfectants can be improved using bis-hydroxyethylamino ethanesulfonate (BES). Calcium phosphate transfection systems are commercially available (e.g., PROFECTION®, Promega Corp., Madison, Wis.). DEAE-dextran transfection may also be used.

Isolated placental cells may also be genetically engineered by microinjection. In certain embodiments, a glass micropipette is guided into the nucleus of cells under a light microscope to inject DNA or RNA.

Placental cells can also be genetically modified using electroporation. In certain embodiments, DNA or RNA is added to a suspension of cultured cells, and the DNA/RNA-cell suspension is placed between two electrodes and subjected to an electrical pulse, causing a transient permeability in the cell's outer membrane that is manifested by the appearance of pores across the membrane.

Liposomal delivery of DNA or RNA to genetically modify the cells can be performed using cationic liposomes, optionally including dioleoyl phosphatidylethanolamine (DOPE) or dioleoyl phosphatidylcholine (DOPC), e.g., LIPOFECTIN® (Life Technologies, Inc.). Other commercially-available delivery systems include EFFECTENE™ (Qiagen), DOTAP (Roche Molecular Biochemicals), FUGENE 6™. (Roche Molecular Biochemicals), and TRANSFECTAM® (Promega).

Viral vectors can be used to genetically alter placental cells by delivery of, e.g., target genes, polynucleotides, antisense molecules, or ribozyme sequences into the cells. Retroviral vectors are effective for transducing rapidly-dividing cells, although a number of retroviral vectors have been developed to effectively transfer DNA into non-dividing cells as well. Packaging cell lines for retroviral vectors are known to those of skill in the art. In certain embodiments, a retroviral DNA vector contains two retroviral LTRs such that a first LTR is located 5′ to the SV40 promoter, which is operationally linked to the target gene sequence cloned into a multicloning site, followed by a 3′ second LTR. Once formed, the retroviral DNA vector is transferred into a packaging cell line using calcium phosphate-mediated transfection, as previously described. Following approximately 48 hours of virus production, the viral vector, now containing the target gene sequence, is harvested. Methods of transfecting cells using lentiviral vectors, recombinant herpes viruses, adenoviral vectors, or alphavirus vectors are known in the art.

Successful transfection or transduction of target cells can be demonstrated using genetic markers, in a technique that is known to those of skill in the art. The green fluorescent protein of Aequorea victoria, for example, has been shown to be an effective marker for identifying and tracking genetically modified hematopoietic cells. Alternative selectable markers include the j-Gal gene, truncated nerve growth factor receptor, or drug selectable markers (including but not limited to NEO, MTX, or hygromycin).

### 5.7.3 Pharmaceutical Compositions

Populations of stimulated isolated placental cells, e.g., PDACs, for example IL-1 β-stimulated PDACs, or populations of cells comprising the stimulated isolated placental cells, can be formulated into pharmaceutical compositions for use in vivo, e.g., in the methods of treatment provided herein. Such pharmaceutical compositions comprise a population of stimulated isolated placental cells, or a population of cells comprising stimulated isolated placental cells, in a pharmaceutically-acceptable carrier, e.g., a saline solution or other accepted physiologically-acceptable solution for in vivo administration. Pharmaceutical compositions comprising the stimulated isolated placental cells described herein can comprise any, or any combination, of the stimulated isolated placental cell populations, or stimulated isolated placental cells, described elsewhere herein. The pharmaceutical compositions can comprise fetal, maternal, or both fetal and maternal stimulated isolated placental cells. The pharmaceutical compositions provided herein can further comprise stimulated isolated placental cells obtained from a single individual or placenta, or from a plurality of individuals or placentae.

The pharmaceutical compositions provided herein can comprise any number of stimulated isolated placental cells. For example, a single unit dose of stimulated isolated placental cells can comprise, in various embodiments, about, at least, or no more than 1×10², 5×10² 1×10³, 5×10³, 1×10⁴, 5×10⁴′ 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more isolated placental cells. In other embodiments, a single unit dose of stimulated isolated placental cells can comprise about, at least, or no more than 1×10²-5×10², 5×10²-1×10³, 1×10³-5×10³, 5×10³-1×10⁴, 1×10⁴-5×10⁴, 5×10⁴-1×10⁵, 1×10⁵-5×10⁵, 5×10⁵-1×10⁶, 1×10⁶-5×10⁶, 5×10⁶-1×10⁷, 1×10⁷-5×10⁷, 5×10⁷-1×10⁸, 1×10⁸-5×10⁸, 5×10⁸-1×10⁹, 1×10⁹-5×10⁹, 5×10⁹-1×10¹⁰, 1×10¹⁰-5×10¹⁰, 5×10¹⁰-1×10¹¹, or 1×10¹¹-5×10¹¹ or more isolated placental cells.

The pharmaceutical compositions provided herein comprise populations of cells that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

The pharmaceutical compositions provided herein can comprise one or more compounds that, e.g., facilitate engraftment (e.g., anti-T-cell receptor antibodies, an immunosuppressant, or the like); stabilizers such as albumin, dextran 40, gelatin, hydroxyethyl starch, plasmalyte, and the like.

When formulated as an injectable solution, in one embodiment, the pharmaceutical composition comprises about 1% to 1.5% HSA and about 2.5% dextran. In a preferred embodiment, the pharmaceutical composition comprises from about 5×10⁶ cells per milliliter to about 2×10⁷ cells per milliliter in a solution comprising 5% HSA and 10% dextran, optionally comprising an immunosuppressant, e.g., cyclosporine A at, e.g., 10 mg/kg.

In other embodiments, the pharmaceutical composition, e.g., a solution, comprises a plurality of cells, e.g., stimulated isolated placental cells, for example, PDACs, e.g., IL-1 β-stimulated PDACs, wherein said pharmaceutical composition comprises between about 1.0±0.3×10⁶ cells per milliliter to about 5.0±1.5×10⁶ cells per milliliter. In other embodiments, the pharmaceutical composition comprises between about 1.5×10⁶ cells per milliliter to about 3.75×10⁶ cells per milliliter. In other embodiments, the pharmaceutical composition comprises between about 1×10⁶ cells/mL to about 50×10⁶ cells/mL, about 1×10⁶ cells/mL to about 40×10⁶ cells/mL, about 1×10⁶ cells/mL to about 30×10⁶ cells/mL, about 1×10⁶ cells/mL to about 20×10⁶ cells/mL, about 1×10⁶ cells/mL to about 15×10⁶ cells/mL, or about 1×10⁶ cells/mL to about 10×10⁶ cells/mL. In certain embodiments, the pharmaceutical composition comprises no visible cell clumps (i.e., no macro cell clumps), or substantially no such visible clumps. As used herein, “macro cell clumps” means an aggregation of cells visible without magnification, e.g., visible to the naked eye, and generally refers to a cell aggregation larger than about 150 microns In some embodiments, the pharmaceutical composition comprises about 2.5%, 3.0%, 3.5%, 4.0%, 4.5%, 5.0%, 5.5%, 6.0%, 6.5%, 7.0%, 7.5% 8.0%, 8.5%, 9.0%, 9.5% or 10% dextran, e.g., dextran-40. In a specific embodiment, said composition comprises about 7.5% to about 9% dextran-40. In a specific embodiment, said composition comprises about 5.5% dextran-40. In certain embodiments, the pharmaceutical composition comprises from about 1% to about 15% human serum albumin (HSA). In specific embodiments, the pharmaceutical composition comprises about 1%, 2%, 3%, 4%, 5%, 65, 75, 8%, 9% 10%, 11%, 12%, 13%, 14% or 15% HSA. In a specific embodiment, said cells have been cryopreserved and thawed. In another specific embodiment, said cells have been filtered through a 70 μM to 100 μM filter. In another specific embodiment, said composition comprises no visible cell clumps. In another specific embodiment, said composition comprises fewer than about 200 cell clumps per 10⁶ cells, wherein said cell clumps are visible only under a microscope, e.g., a light microscope. In another specific embodiment, said composition comprises fewer than about 150 cell clumps per 10⁶ cells, wherein said cell clumps are visible only under a microscope, e.g., a light microscope. In another specific embodiment, said composition comprises fewer than about 100 cell clumps per 10⁶ cells, wherein said cell clumps are visible only under a microscope, e.g., a light microscope.

In a specific embodiment, the pharmaceutical composition comprises about 1.0±0.3×10⁶ cells per milliliter, about 5.5% dextran-40 (w/v), about 10% HSA (w/v), and about 5% DMSO (v/v).

In other embodiments, the pharmaceutical composition comprises a plurality of stimulated cells, e.g., stimulated PDACs, for example IL-1 β-stimulated PDACs in a solution comprising 10% dextran-40, wherein the pharmaceutical composition comprises between about 1.0 0.3×10⁶ cells per milliliter to about 5.0±1.5×10⁶ cells per milliliter, and wherein said composition comprises no cell clumps visible with the unaided eye (i.e., comprises no macro cell clumps). In some embodiments, the pharmaceutical composition comprises between about 1.5×10⁶ cells per milliliter to about 3.75×10⁶ cells per milliliter. In a specific embodiment, said cells have been cryopreserved and thawed. In another specific embodiment, said cells have been filtered through a 7 μM to 10 μM filter. In another specific embodiment, said composition comprises fewer than about 200 micro cell clumps (that is, cell clumps visible only with magnification) per 10⁶ cells. In another specific embodiment, the pharmaceutical composition comprises fewer than about 150 micro cell clumps per 10⁶ cells. In another specific embodiment, the pharmaceutical composition comprises fewer than about 100 micro cell clumps per 10⁶ cells. In another specific embodiment, the pharmaceutical composition comprises less than 15%, 14%, 13%, 12%, 11%, 10%, 9%, 8%, 7%, 6%, 5%, 4% 3% or 2% DMSO, or less than 1%, 0.9%, 0.8%, 0.7%, 0.6%, 0.5%, 0.4%, 0.3%, 0.2%, or 0.1% DMSO.

Further provided herein are compositions comprising stimulated cells, e.g., stimulated PDACs, for example IL-1 β-stimulated PDACs wherein said compositions are produced by one of the methods disclosed herein. For example, in one embodiment, the pharmaceutical composition comprises cells, wherein the pharmaceutical composition is produced by a method comprising filtering a solution comprising placental cells, e.g., placental stem cells or placental multipotent cells, to form a filtered cell-containing solution; diluting the filtered cell-containing solution with a first solution to about 1 to 50×10⁶, 1 to 40×10⁶, 1 to 30×10⁶, 1 to 20×10⁶, 1 to 15×10⁶, or 1 to 10×10⁶ cells per milliliter, e.g., prior to cryopreservation; and diluting the resulting filtered cell-containing solution with a second solution comprising dextran, but not comprising human serum albumin (HSA) to produce said composition. In certain embodiments, said diluting is to no more than about 15×10⁶ cells per milliliter. In certain embodiments, said diluting is to no more than about 10 3×10⁶ cells per milliliter. In certain embodiments, said diluting is to no more than about 7.5×10⁶ cells per milliliter. In other certain embodiments, if the filtered cell-containing solution, prior to the dilution, comprises less than about 15×10⁶ cells per milliliter, filtration is optional. In other certain embodiments, if the filtered cell-containing solution, prior to the dilution, comprises less than about 10 3×10⁶ cells per milliliter, filtration is optional. In other certain embodiments, if the filtered cell-containing solution, prior to the dilution, comprises less than about 7.5×10⁶ cells per milliliter, filtration is optional.

In a specific embodiment, the stimulated cells, e.g., stimulated PDACs, for example IL-1 β-stimulated PDACs are cryopreserved between said diluting with a first dilution solution and said diluting with said second dilution solution. In another specific embodiment, the first dilution solution comprises dextran and HSA. The dextran in the first dilution solution or second dilution solution can be dextran of any molecular weight, e.g., dextran having a molecular weight of from about 10 kDa to about 150 kDa. In some embodiments, said dextran in said first dilution solution or said second solution is about 2.5%, 3.0%, 3.5%, 4.0%, 4.5%, 5.0%, 5.5%, 6.0%, 6.5%, 7.0%, 7.5% 8.0%, 8.5%, 9.0%, 9.5% or 10% dextran. In another specific embodiment, the dextran in said first dilution solution or said second dilution solution is dextran-40. In another specific embodiment, the dextran in said first dilution solution and said second dilution solution is dextran-40. In another specific embodiment, said dextran-40 in said first dilution solution is 5.0% dextran-40. In another specific embodiment, said dextran-40 in said first dilution solution is 5.5% dextran-40. In another specific embodiment, said dextran-40 in said second dilution solution is 10% dextran-40. In another specific embodiment, said HSA in said solution comprising HSA is 1 to 15% HSA. In another specific embodiment, said HSA in said solution comprising HSA is about 1%, 2%, 3%, 4%, 5%, 6%, 7%, 8%, 9%, 10%, 11%, 12%, 13%, 14% or 15% HSA. In another specific embodiment, said HSA in said solution comprising HSA is 10% HSA. In another specific embodiment, said first dilution solution comprises HSA. In another specific embodiment, said HSA in said first dilution solution is 10% HSA. In another specific embodiment, said first dilution solution comprises a cryoprotectant. In another specific embodiment, said cryoprotectant is DMSO. In another specific embodiment, said dextran-40 in said second dilution solution is about 10% dextran-40. In another specific embodiment, said composition comprising cells comprises about 7.5% to about 9% dextran. In another specific embodiment, the pharmaceutical composition comprises from about 1.0 0.3×10⁶ cells per milliliter to about 5.0±1.5×10⁶ cells per milliliter. In another specific embodiment, the pharmaceutical composition comprises from about 1.5×10⁶ cells per milliliter to about 3.75×10⁶ cells per milliliter.

In another embodiment, the pharmaceutical composition is made by a method comprising (a) filtering a cell-containing solution comprising stimulated placental cells, e.g., stimulated PDACs, for example IL-1 β-stimulated PDACs, prior to cryopreservation to produce a filtered cell-containing solution; (b) cryopreserving the cells in the filtered cell-containing solution at about 1 to 50×10⁶, 1 to 40×10⁶, 1 to 30×10⁶, 1 to 20×10⁶, 1 to 15×10⁶, or 1 to 10×10⁶ cells per milliliter; (c) thawing the cells; and (d) diluting the filtered cell-containing solution about 1:1 to about 1:11 (v/v) with a dextran-40 solution. In certain embodiments, if the number of cells is less than about 10 3×10⁶ cells per milliliter prior to step (a), filtration is optional. In another specific embodiment, the cells in step (b) are cryopreserved at about 10±3×10⁶ cells per milliliter. In another specific embodiment, the cells in step (b) are cryopreserved in a solution comprising about 5% to about 10% dextran-40 and HSA. In certain embodiments, said diluting in step (b) is to no more than about 15×10⁶ cells per milliliter.

In another embodiment, the pharmaceutical composition is made by a method comprising: (a) suspending stimulated placental cells, e.g., stimulated PDACs, for example IL-1 β-stimulated PDACs, in a 5.5% dextran-40 solution that comprises 10% HSA to form a cell-containing solution; (b) filtering the cell-containing solution through a 70 μM filter; (c) diluting the cell-containing solution with a solution comprising 5.5% dextran-40, 10% HSA, and 5% DMSO to about 1 to 50×10⁶, 1 to 40×10⁶, 1 to 30×10⁶, 1 to 20×10⁶, 1 to 15×10⁶, or 1 to 10×10⁶ cells per milliliter; (d) cryopreserving the cells; (e) thawing the cells; and (f) diluting the cell-containing solution 1:1 to 1:11 (v/v) with 10% dextran-40. In certain embodiments, said diluting in step (c) is to no more than about 15×10⁶ cells per milliliter. In certain embodiments, said diluting in step (c) is to no more than about 10 3×10⁶ cells/mL. In certain embodiments, said diluting in step (c) is to no more than about 7.5×10⁶ cells/mL.

In another embodiment, the composition comprising stimulated cells is made by a method comprising: (a) centrifuging a plurality of cells to collect the cells; (b) resuspending the cells in 5.5% dextran-40; (c) centrifuging the cells to collect the cells; (d) resuspending the cells in a 5.5% dextran-40 solution that comprises 10% HSA; (e) filtering the cells through a 70 μM filter; (f) diluting the cells in 5.5% dextran-40, 10% HSA, and 5% DMSO to about 1 to 50×10⁶, 1 to 40×10⁶, 1 to 30×10⁶, 1 to 20×10⁶, 1 to 15×10⁶, or 1 to 10×10⁶ cells per milliliter; (g) cryopreserving the cells; (h) thawing the cells; and (i) diluting the cells 1:1 to 1:11 (v/v) with 10% dextran-40. In certain embodiments, said diluting in step (f) is to no more than about 15×10⁶ cells per milliliter. In certain embodiments, said diluting in step (f) is to no more than about 3×10⁶ cells/mL. In certain embodiments, said diluting in step (f) is to no more than about 7.5×10⁶ cells/mL. In other certain embodiments, if the number of cells is less than about 10±3×10⁶ cells per milliliter, filtration is optional.

The compositions, e.g., pharmaceutical compositions comprising the stimulated isolated placental cells described herein can comprise any of the isolated placental cells described herein.

Other injectable formulations, suitable for the administration of cellular products, may be used.

In one embodiment, the pharmaceutical composition comprises stimulated isolated placental cells that are substantially, or completely, non-maternal in origin, that is, have the fetal genotype; e.g., at least about 90%, 95%, 98%, 99% or about 100% are non-maternal in origin. For example, in one embodiment a pharmaceutical composition comprises a population of stimulated isolated placental cells that are CD200⁺ and HLA-G⁻; CD73⁺, CD105⁺, and CD200⁺; CD200⁺ and OCT-4⁺; CD73⁺, CD105⁺ and HLA-G⁻; CD73⁺ and CD105⁺ and facilitate the formation of one or more embryoid-like bodies in a population of placental cells comprising said population of isolated placental cell when said population of placental cells is cultured under conditions that allow the formation of an embryoid-like body; or OCT-4⁺ and facilitate the formation of one or more embryoid-like bodies in a population of placental cells comprising said population of isolated placental cell when said population of placental cells is cultured under conditions that allow the formation of an embryoid-like body; or a combination of the foregoing, wherein at least 70%, 80%, 90%, 95% or 99% of said isolated placental cells are non-maternal in origin. In another embodiment, a pharmaceutical composition comprises a population of isolated placental cells that are CD10⁺, CD105⁺ and CD34⁻; CD10⁺, CD105⁺, CD200⁺ and CD34⁻; CD10⁺, CD105⁺, CD200⁺, CD34⁻ and at least one of CD90⁺ or CD45⁻; CD10⁺, CD90⁺, CD105⁺, CD200⁺, CD34⁻ and CD45⁻; CD10⁺, CD90⁺, CD105⁺, CD200⁺, CD34⁻ and CD45⁻; CD200⁺ and HLA-G⁻; CD73⁺, CD105⁺, and CD200⁺; CD200⁺ and OCT-4⁺; CD73⁺, CD105⁺ and HLA-G⁻; CD73⁺ and CD105⁺ and facilitate the formation of one or more embryoid-like bodies in a population of placental cells comprising said isolated placental cells when said population of placental cells is cultured under conditions that allow the formation of an embryoid-like body; OCT-4⁺ and facilitate the formation of one or more embryoid-like bodies in a population of placental cells comprising said isolated placental cells when said population of placental cells is cultured under conditions that allow the formation of an embryoid-like body; or one or more of CD117⁻, CD133⁻, KDR⁻, CD80⁻, CD86⁻, HLA-A,B,C⁺, HLA-DP,DQ,DR⁻ and/or PDL1⁺; or a combination of the foregoing, wherein at least 70%, 80%, 90%, 95% or 99% of said isolated placental cells are non-maternal in origin. In a specific embodiment, the pharmaceutical composition additionally comprises a stem cell that is not obtained from a placenta. In another embodiment, the isolated placental stem cells are stimulated with one or more pro-inflammatory cytokines. In specific embodiments, the pro-inflammatory cytokines comprise one or more of IL-1 α, IL-1 β, IL-6, IL-8, IL-18, TNF-α, and INF-γ. In other specific embodiments, the pro-inflammatory cytokine is IL-1β.

Isolated placental cells in the compositions, e.g., pharmaceutical compositions, provided herein, can comprise placental cells derived from a single donor, or from multiple donors. The isolated placental cells can be completely HLA-matched to an intended recipient, or partially or completely HLA-mismatched.

### 5.7.4 Matrices Comprising Isolated Placental Cells

Further provided herein are compositions comprising matrices, hydrogels, scaffolds, and the like that comprise a placental cell, or a population of isolated placental cells. Such compositions can be used in the place of, or in addition to, cells in liquid suspension.

The stimulated isolated placental cells described herein can be seeded onto a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H₂O) amniotic membrane, chorionic membrane, substantially dry chorionic membrane, substantially dry amniotic and chorionic membrane, and the like. Preferred placental biomaterials on which isolated placental cells can be seeded are described in Hariri, U.S. Application Publication No. 2004/0048796, the disclosure of which is incorporated herein by reference in its entirety.

The stimulated isolated placental cells described herein can be suspended in a hydrogel solution suitable for, e.g., injection. Suitable hydrogels for such compositions include self-assembling peptides, such as RAD16. In one embodiment, a hydrogel solution comprising the cells can be allowed to harden, for instance in a mold, to form a matrix having cells dispersed therein for implantation. Isolated placental cells in such a matrix can also be cultured so that the cells are mitotically expanded prior to implantation. The hydrogel is, e.g., an organic polymer (natural or synthetic) that is cross-linked via covalent, ionic, or hydrogen bonds to create a three-dimensional open-lattice structure that entraps water molecules to form a gel. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix is biodegradable.

In some embodiments, the formulation comprises an in situ polymerizable gel (see, e.g., U.S. Patent Application Publication 2002/0022676, the disclosure of which is incorporated herein by reference in its entirety; Anseth et al., J. Control Release, 78(1-3): 199-209 (2002); Wang et al., Biomaterials, 24(22):3969-80 (2003).

In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

In a specific embodiment, the matrix is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling. In another preferred embodiment the cells of the invention are seeded onto foam scaffolds that may be composite structures. In addition, the three-dimensional framework may be molded into a useful shape, such as a specific structure in the body to be repaired, replaced, or augmented. Other examples of scaffolds that can be used include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(F-caprolactone)/poly(glycolic acid) (PCL/PGA) copolymer, formed by processes such as freeze-drying, or lyophilization (see, e.g., U.S. Pat. No. 6,355,699), can also be used as scaffolds.

The stimulated isolated placental cells described herein or co-cultures thereof can be seeded onto a three-dimensional framework or scaffold and implanted in vivo. Such a framework can be implanted in combination with any one or more growth factors, cells, drugs or other components that, e.g., stimulate tissue formation.

Examples of scaffolds that can be used include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(F-caprolactone)/poly(glycolic acid) (PCL/PGA) copolymer, formed by processes such as freeze-drying, or lyophilization (see, e.g., U.S. Pat. No. 6,355,699), can also be used as scaffolds.

In another embodiment, stimulated isolated placental cells can be seeded onto, or contacted with, a felt, which can be, e.g., composed of a multifilament yarn made from a bioabsorbable material such as PGA, PLA, PCL copolymers or blends, or hyaluronic acid.

The stimulated isolated placental cells provided herein can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1M acetic acid followed by incubation in polylysine, PBS, and/or collagen, prior to inoculation of the cells in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the scaffold comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™ (The Polymer Technology Group, Inc., Berkeley, Calif.). The scaffold can also comprise anti-thrombotic agents such as heparin; the scaffolds can also be treated to alter the surface charge (e.g., coating with plasma) prior to seeding with stimulated isolated placental cells.

The stimulated placental cells (e.g., stimulated PDACs, for example IL-1 β-stimulated PDACs) provided herein can also be seeded onto, or contacted with, a physiologically-acceptable ceramic material including, but not limited to, mono-, di-, tri-, alpha-tri-, beta-tri-, and tetra-calcium phosphate, hydroxyapatite, fluoroapatites, calcium sulfates, calcium fluorides, calcium oxides, calcium carbonates, magnesium calcium phosphates, biologically active glasses such as BIOGLASS®, and mixtures thereof. Porous biocompatible ceramic materials currently commercially available include SURGIBONE® (CanMedica Corp., Canada), ENDOBON® (Merck Biomaterial France, France), CEROS® (Mathys, AG, Bettlach, Switzerland), and mineralized collagen bone grafting products such as HEALOS™ (DePuy, Inc., Raynham, Mass.) and VITOSS®, RHAKOSS™, and CORTOSS® (Orthovita, Malvern, Pa.). The framework can be a mixture, blend or composite of natural and/or synthetic materials.

In one embodiment, the stimulated isolated placental cells are seeded onto, or contacted with, a suitable scaffold at about 0.5×10⁶ to about 8×10⁶ cells/mL.

### 5.8 Kits

In another aspect, provided herein are kits, suitable for the treatment of an individual who has a disease or disorder of the circulatory system, comprising, in a container separate from remaining kit contents, stimulated PDACs, e.g., the cells described in Section 5.2, above (e.g., IL-1 β-stimulated PDACs), and instructions for use. Preferably, the stimulated placental cells are provided in a pharmaceutically-acceptable solution, e.g., a solution suitable for intralesional administration or a solution suitable for intravenous administration. In certain embodiments, the stimulated placental stem cells or stimulated placental multipotent cells are any of the CD10⁺, CD34⁻, CD105⁺ placental cells described herein, e.g., CD10⁺, CD34⁻, CD105⁺, CD200⁺ placental cells or CD10⁺, CD34⁻, CD45⁻, CD90⁺, CD105⁺, CD200⁺ placental cells.

In certain embodiments, the kits comprise one or more components that facilitate delivery of the stimulated placental cells to the individual. For example, in certain embodiments, the kit comprises components that facilitate intralesional delivery of the stimulated placental cells to the individual. In such embodiments, the kit can comprise, e.g., syringes and needles suitable for delivery of cells to the individual, and the like. In such embodiments, the stimulated placental cells may be contained in the kit in a bag, or in one or more vials. In certain other embodiments, the kit comprises components that facilitate intravenous or intra-arterial delivery of the stimulated placental cells to the individual. In such embodiments, the stimulated placental cells may be contained, e.g., within a bottle or bag (for example, a blood bag or similar bag able to contain up to about 1.5 L solution comprising the cells), and the kit additionally comprises tubing and needles suitable for the delivery of cells to the individual.

Additionally, the kit may comprise one or more compounds that reduce pain or inflammation in the individual (e.g., an analgesic, steroidal or non-steroidal anti-inflammatory compound, or the like. The kit may also comprise an antibacterial or antiviral compound (e.g., one or more antibiotics), a compound to reduce anxiety in the individual (e.g., alaprazolam), a compound that reduces an immune response in the individual (e.g., cyclosporine A), an antihistamine (diphenhydramine, loratadine, desloratadine, quetiapine, fexofenadine, cetirizine, promethazine, chlorepheniramine, levocetirizine, cimetidine, famotidine, ranitidine, nizatidine, roxatidine, lafutidine, or the like).

Additionally, the kit can comprise disposables, e.g., sterile wipes, disposable paper goods, gloves, or the like, which facilitate preparation of the individual for delivery, or which reduce the likelihood of infection in the individual as a result of the administration of the stimulated placental cells.

### 6. EXAMPLES

### 6.1 Example 1: The Effect of Secreted PDAC Factors on Vascular Cell Survival, Proliferation, and Morphology

### 6.1.1 PDAC Growth Factor and Cytokine Secretion

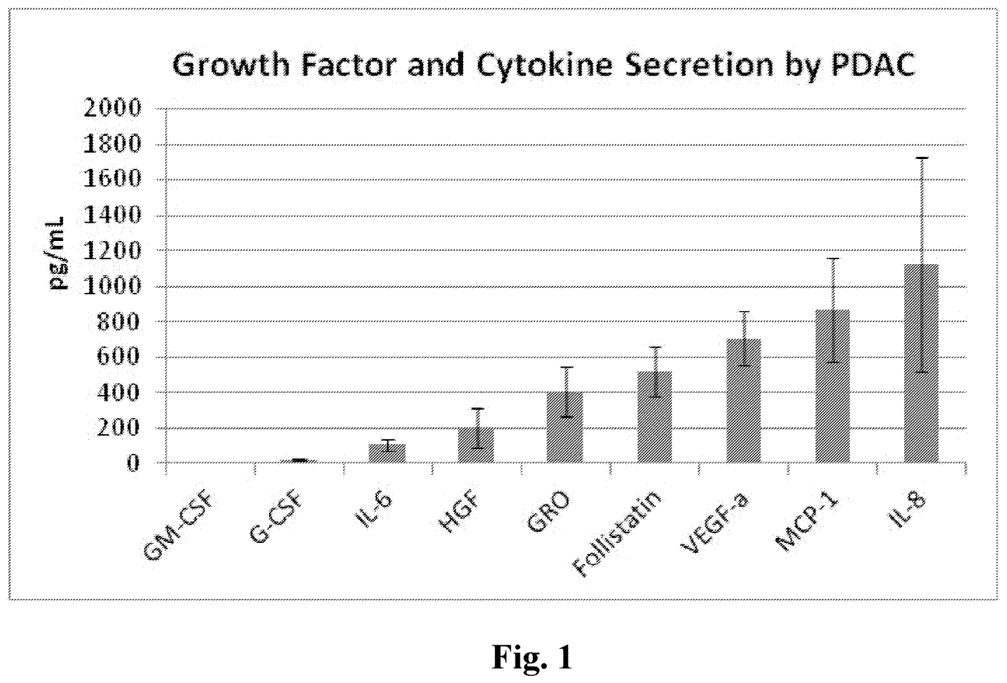

Frozen aliquots of placenta-derived adherent stem cells (PDACs) were thawed and expanded in complete growth medium (Dulbecco's Modified Eagle's Medium [DMEM] supplemented with 10% fetal calf serum). Cultured PDACs were then incubated in serum-free DMEM for 24 hours and the PDAC cell-conditioned media samples (P-CM) were collected. Concentrations of secreted trophic factors in P-CM were determined using two Milliplex® MAP Immunoassay Panels (Human Angiogenesis/Growth Factor Panel 1 and Human Cytokine/Chemokine Panel) (Millipore). The results of this experiment are shown in FIG. 1. P-CM was determined to contain detectable levels of various trophic angiogenic factors, including IL-8, MCP-1, VEGF-a, Follistatin, GRO, HGF, and IL-6, indicating that cultured PDACs are capable of secreting angiogenic factors.

### 6.1.2 Secreted PDAC Factors Alter HUVEC Survival and Tube Formation

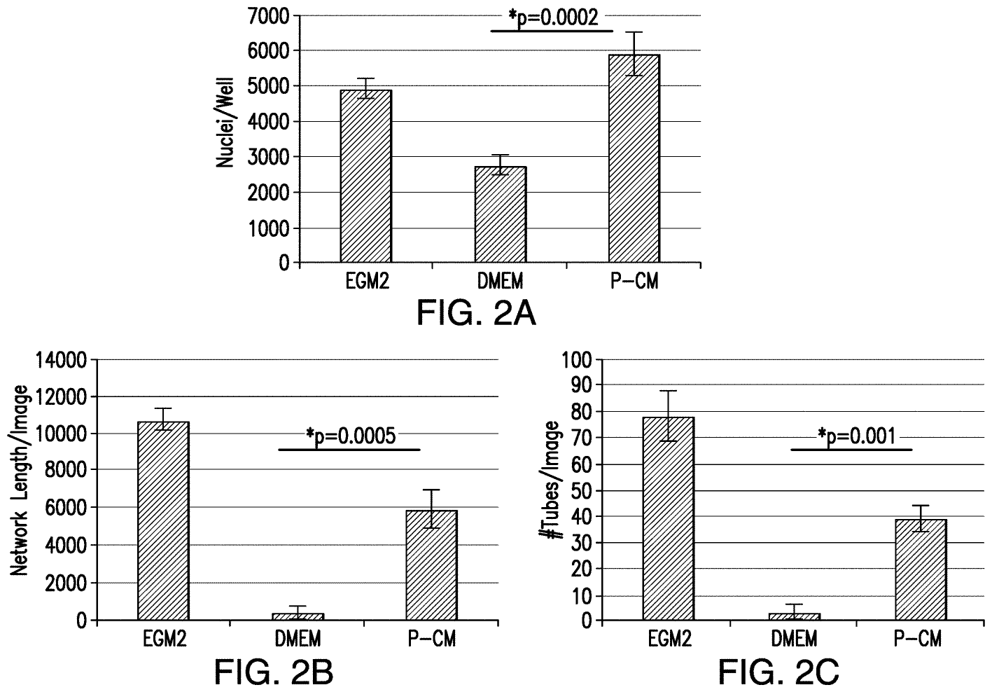

Human vascular endothelial cells (HUVECs) were thawed and plated on Fibronectin-coated plates in complete Endothelial Growth Medium (EGM™-2) (Lonza) overnight. HUVECs were cultured in serum-free Endothelial Basal Medium (EBM™-2) (Lonza) for 6 hours followed by incubation in PDAC cell-conditioned media samples (P-CM) for 16 hours at a P-CM concentration of 0.104 mL/cm² of the culture flask. Treated HUVEC cell viability was determined by Hoechst staining (Invitrogen) followed by quantitative image analysis using and InCell Analyzer 2000 (GE Healthcare). As shown in FIG. 2A, when compared to HUVECs treated with DMEM alone, HUVECs incubated with P-CM had a significantly greater proliferation/survival rate.

In a separate experiment, HUVECs were serum-starved in EBM™-2 for 3 hours and harvested. HUVECs were replated on pre-coated Growth Factor-Reduced Cultrex (Trevigen) plates either in the presence of P-CM or DMEM alone. Re-plated HUVECs were then analyzed on an IncuCyte ZOOM (Essen Bioscience) for 16 hours to determine the extent of HUVEC tube formation. Images were analyzed using ImageJ using the angiogenesis analyzer toolkit. P-CM treated HUVECs formed significantly longer networks (FIG. 2B) and significantly more tubes than the control HUVEcs (FIG. 2C). These data indicate that P-CM promotes tube formation and branching in vascular cell models of angiogenesis.

### 6.2 Example 2: Secreted PDAC Factors Affect HUVEC Cell Signaling and Expression

### 6.2.1 PDAC Conditioned Medium Alters HUVEC Signaling Pathways

HUVECs were thawed and cultured for two days as described in Section 6.1, Supra. HUVECs were then serum-starved for 6 hours in serum-free EBM™-2, followed by culturing in the presence or absence of P-CM for 5, 15, or 30 minutes. Effects of P-CM on HUVEC cell signaling were evaluated using Milliplex® MAP Multi-pathway Cell Signaling Multiplex Analysis (EMD Millipore).

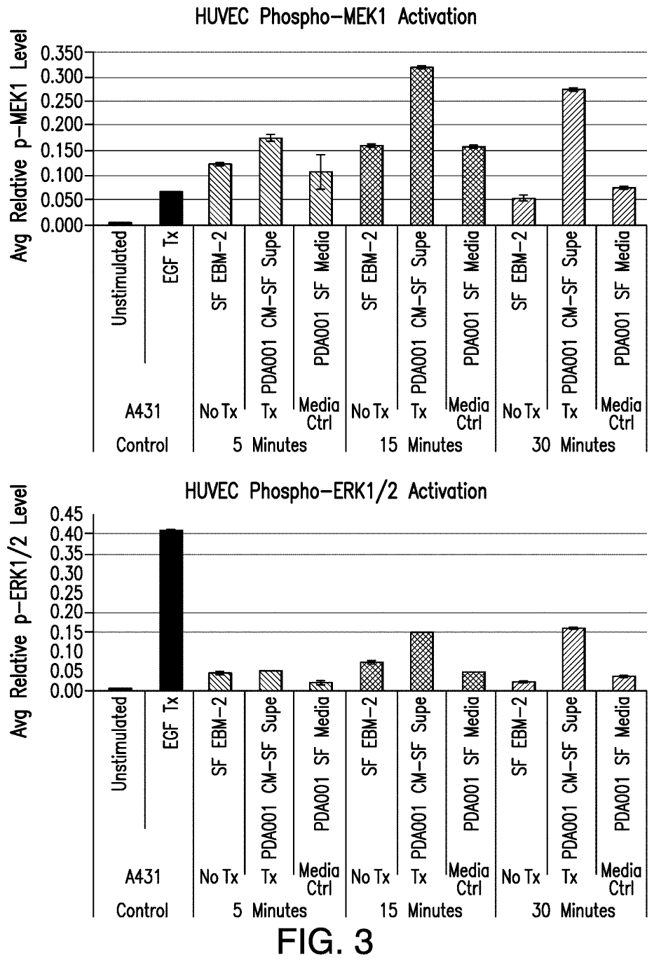

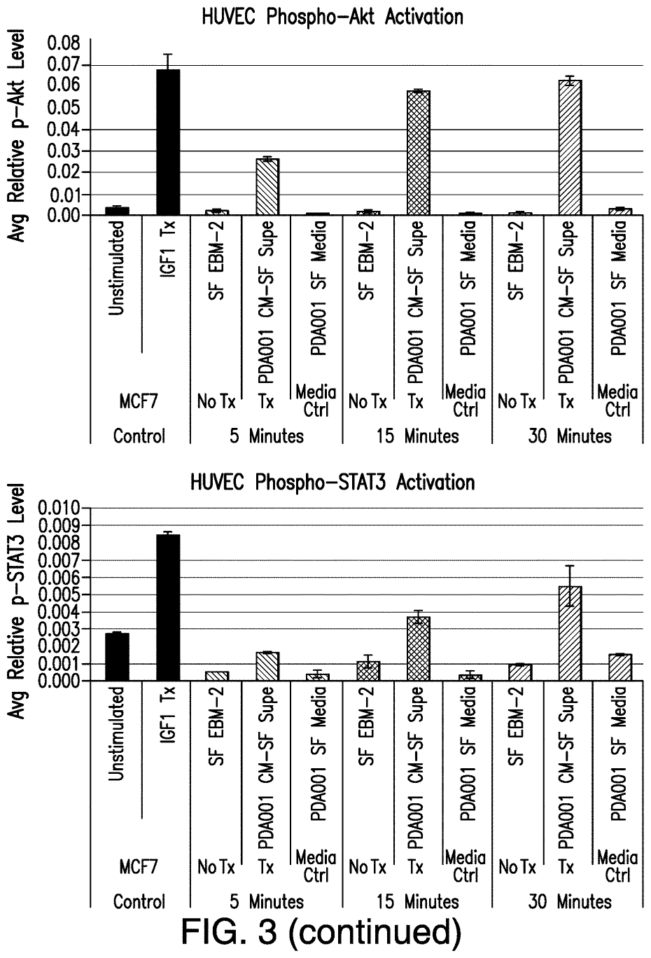

As shown in FIG. 3, HUVECs treated with P-CM (“Tx” lanes) showed dramatically higher levels of phosphorylated MEK, phosphorylated ERK1/2, phosphorylated Akt, and phosphorylated STAT3 levels at all time points tested as compared to culture medium controls (“Media Ctrl” lanes). These results indicate that vascular cells treated with PDAC conditioned medium can promote distinct cell signaling pathways known to be involved in cellular proliferation (MEK1, ERK1/2), survival (Akt), and branching (STAT3).

### 6.2.2 PDAC Conditioned Medium Alters HUVEC Gene Expression

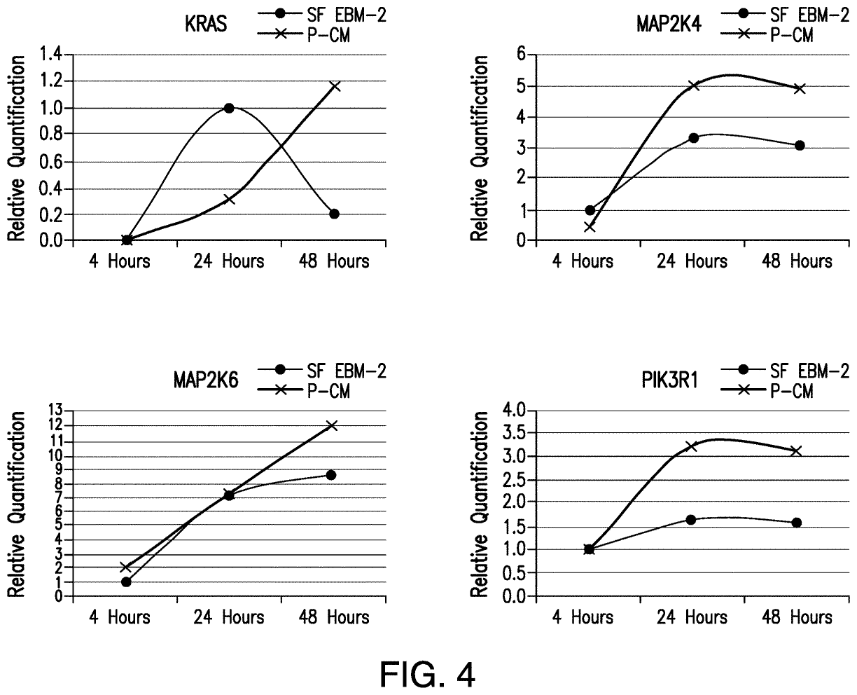

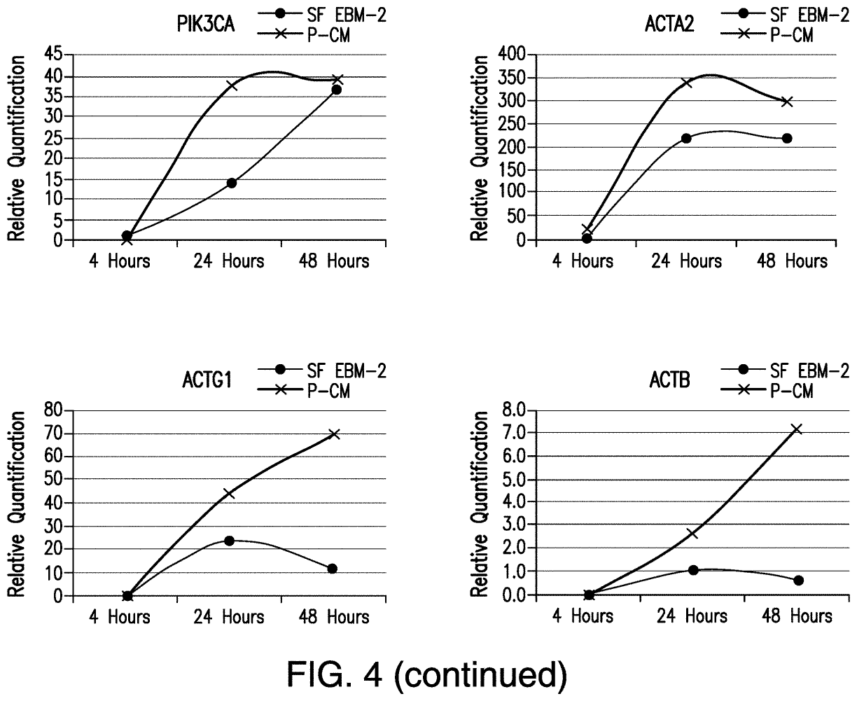

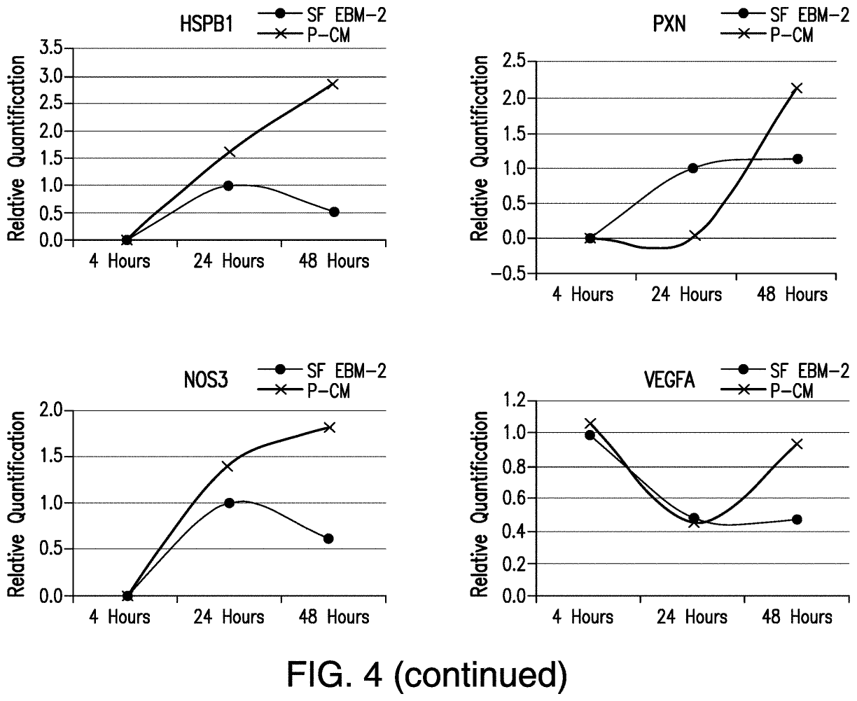

HUVECs were cultured and serum-starved as in Section 6.2.1, Supra. HUVECs were then cultured in the presence of P-CM for 4, 24, or 48 hours, and collected for analysis. VEGF gene expression was measured using Taqman Human VEGF Pathway Arrays and the QuantStudio 12K Real-Time PCR System (Life Technologies) according to the manufacturer's protocol. As shown in FIG. 4, culturing HUVECs in P-CM resulted in the induction of numerous genes over the 48-hour time course. Genes involved in signal transduction, proliferation, and survival (top row); cellular motility, structure, and integrity (middle row); and nitric oxide synthesis and angiogenesis (bottom row) were all increased in P-CM-treated HUVECs (“P-CM”) relative to medium-treated controls (“SF EBM-2”). The genes shown in FIG. 4 are listed below in Table 1 according to their function.

TABLE 1
HUVEC Genes Upregulated by PDAC
Conditioned Culture Medium

| Signal Transduction, | Motility, | | |
|---|---|---|---|
| Proliferation | Structure | | Nitric Oxide |
| and Survival | and Integrity | Angiogenesis | Synthesis |
| KRAS | ACTA2 | VEGFA | NOS3 |
| MAP2K4 | ACTG1 | | |
| MAP2K6 | ACTB | | |
| PIK3R1 | HSPB1 | | |
| PIK3CA | PXN | | |

### 6.3 Example 3: IL-1 Beta Effects on Secreted PDAC Trophic Factors

PDACs were cultured in growth medium for 24 hours. IL-10 was added to the growth medium at concentrations ranging from 10 pg/mL to 10,000 pg/mL, and secreted PDAC factors were measured using two Milliplex® MAP Immunoassay Panels (Human Angiogenesis/Growth Factor Panel 1 and Human Cytokine/Chemokine Panel) (Millipore).

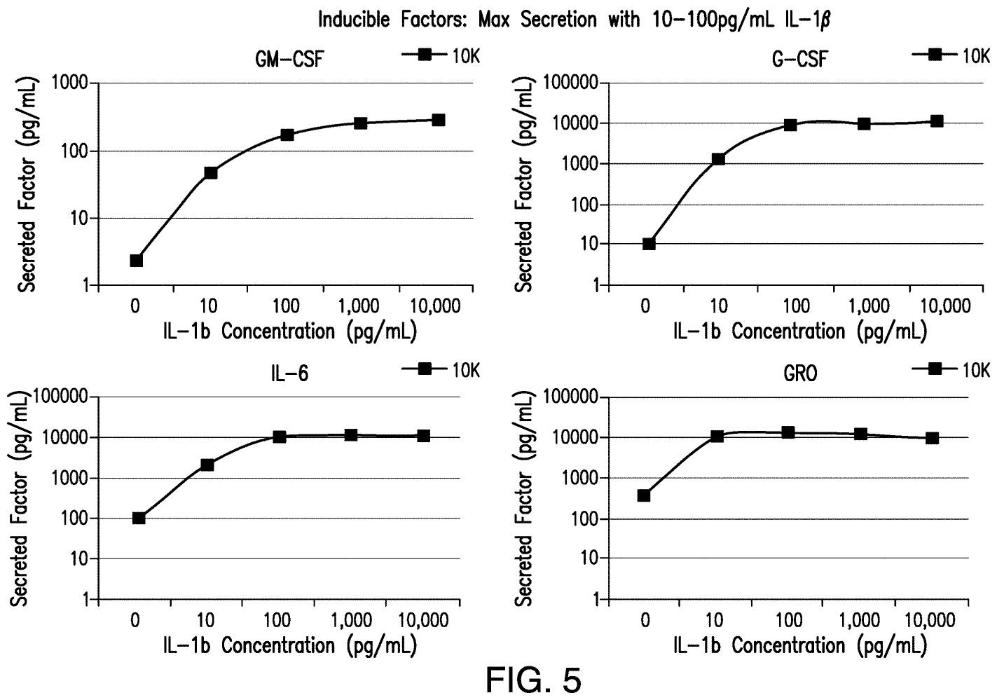

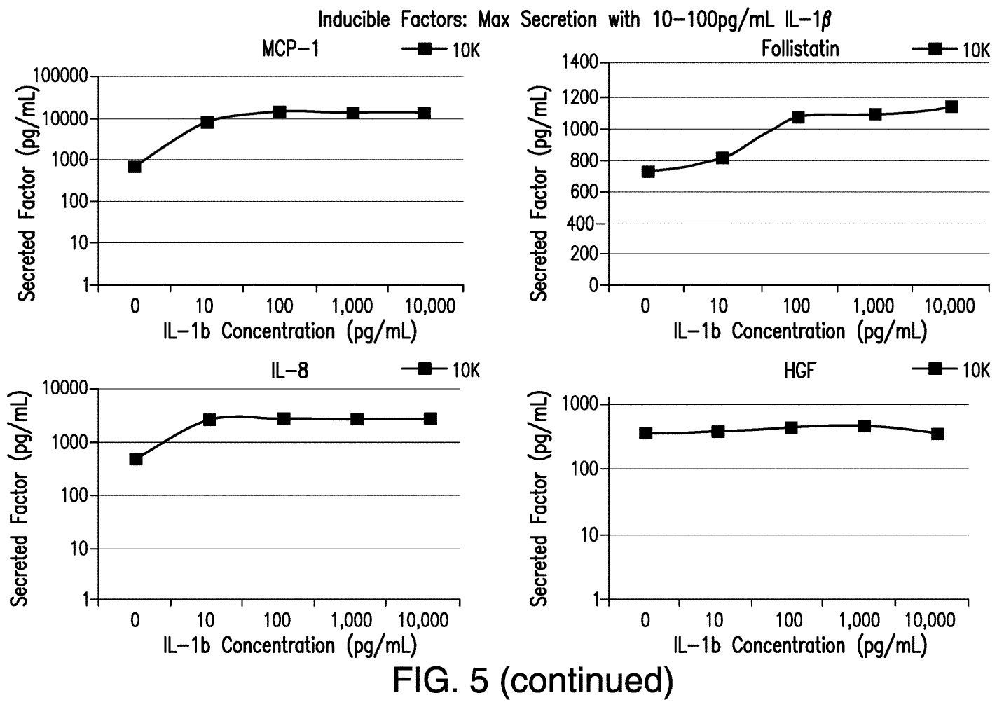

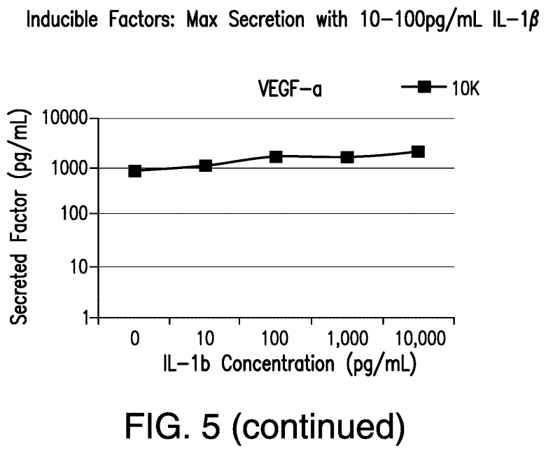

As shown in FIG. 5, PDACs stimulated with IL-10 produced several trophic factors in a dose-dependent manner. These factors include GM-CSF, G-CSF, IL-6, GRO, MCP-1, Follistatin, and IL-8. Conversely, the production of several other trophic factors was not changed after stimulation of PDACs with IL-10, indicating that IL-10 promotes the secretion of a specific subset of trophic factors in cultured PDACs.

### 6.4 Example 4: IL-1 Beta-Stimulated PDAC Conditioned Medium Alters HUVEC Cell Signaling

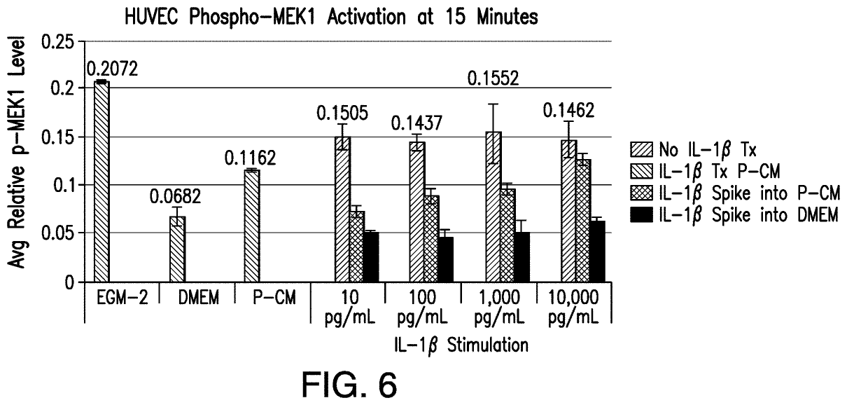

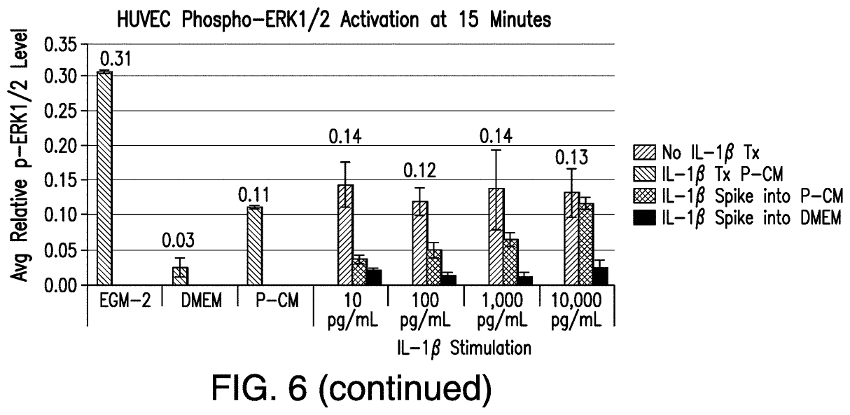

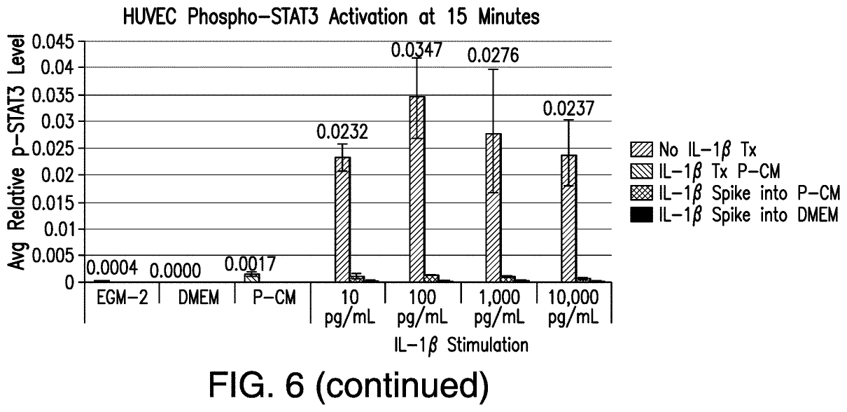

As shown in FIG. 6, HUVECs cultured in the presence of IL-I-stimulated P-CM exhibited higher levels of signaling as compared to cells cultured in the presence of DMEM alone or unstimulated P-CM. Importantly, P-CM spiked with IL-10 also resulted in lower signaling than IL-I-stimulated P-CM, suggesting that the effects of stimulated P-CM on HUVECs are not directly through IL-10. Similar trends were observed for cell signaling pathways involved in proliferation (MEK1, ERK1/2) and branching morphology (STAT3). Together, these data suggest that stimulation of PDACs with pro-inflammatory cytokines can promote proliferative and morphological signaling pathways in endothelial cells.

### 6.5 Example 5: IL-1 Beta-Stimulated Effects Signal Through the Hepatic Growth Factor Pathway

PDACs secrete numerous soluble factors that may contribute to the proliferative effects observed for P-CM. One of these factors, Hepatic Growth Factor (HGF), is constitutively secreted by PDACs. Additionally, HUVECs express HGF Receptor (HGFR, also known as c-Met). To determine the role of the HGF signaling pathway in the pro-angiogenic effects of P-CM, IL-1β-stimulated P-CM was incubated with 500 ng/mL anti-HGF neutralizing antibody for 40 minutes (Abcam) prior to being added to serum-starved HUVECs. Similarly, HUVECs were incubated with 20 nM PHA665752, an inhibitor of HGFR/c-Met, during the six hour serum starvation step as described in Section 6.2.1, Supra.

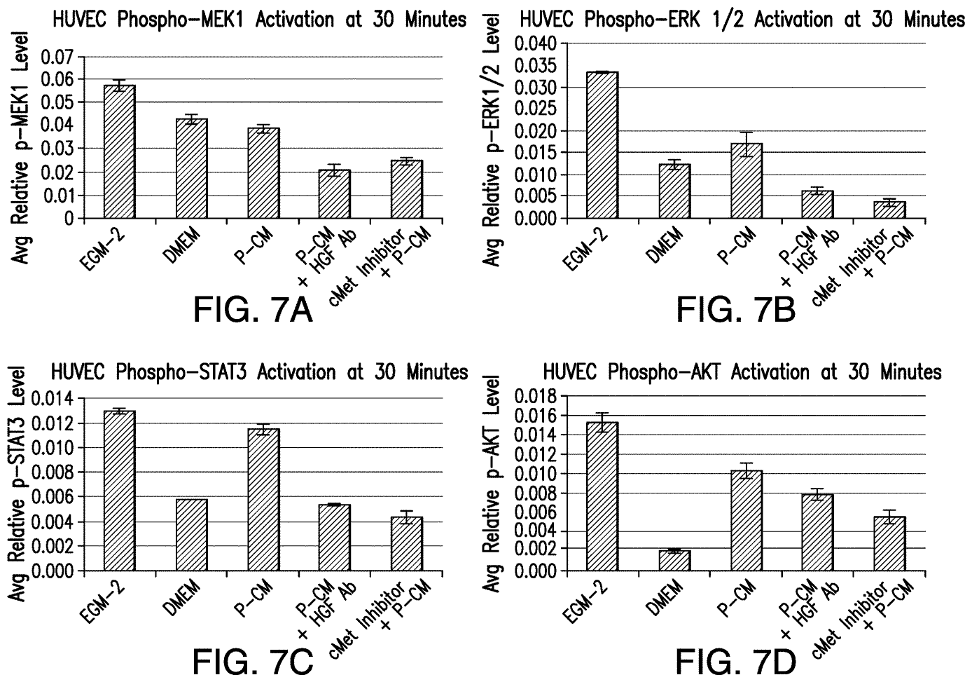

As shown in FIG. 7, the blockade of HGF signaling with either anti-HGF neutralizing antibody or PHA66572 completely reversed the P-CM-induced increase in the MEK, ERK1/2, and STAT3 signaling pathways (FIGS. 7A-C), and partially reversed this effect in the Akt signaling pathway (FIG. 7D). These data indicate that the P-CM-mediated induction of proliferative and angiogenic pathways in cultured HUVECs is at least partially mediated through the Hepatic Growth Factor signaling pathway.

### 6.6 Example 6: Diabetic Foot Ulcer Treatment Protocol

Subjects having diabetic foot ulcer (DFU) with peripheral arterial disease (PAD), aged 18-80, are treated with CD10⁺, CD34⁻, CD105⁺, CD200⁺ placental stem cells that have been stimulated with IL-10. The IL-1β-stimulated placental stem cells are administered intramuscularly on days 1 (the first day of treatment) and 8 at the following doses: (i): 3×10⁶ IL-1β-stimulated CD10⁺, CD34⁻, CD105⁺, CD200⁺ placental stem cells; (ii): 1×10⁷IL-1β-stimulated CD10⁺, CD34⁻, CD105⁺, CD200⁺ placental stem cells; or (iii) 3×10⁷ IL-1β-stimulated CD10⁺, CD34⁻, CD105⁺, CD200⁺ placental stem cells.

Clinical Endpoints

A primary clinical endpoint for efficacy of IL-1-stimulated CD10⁺, CD34⁻, CD105⁺, CD200⁺ placental stem cells for treating DFU can be closure of the DFU or DFUs being treated. Ulcer closure can be represented by skin closure without drainage or need for dressing. Complete closure can be represented by retention of ulcer closure for at least four weeks following determination of closure. Ulcer closure can be assessed at three months following treatment with the placental stem cells.

Other clinical endpoints for efficacy of IL-1β-stimulated CD10⁺, CD34⁻, CD105⁺, CD200⁺ placental stem cells for treating DFU can include: (i) reduction of the frequency and severity of adverse events, which can be assessed up to 24-months following treatment; (ii) time to ulcer closure, which can be assessed at six months following treatment; (ii) improvement in ankle brachial index (ABI), which can be assessed at six months following treatment; (iii) improvement in toe brachial index (TBI), which can be assessed at six months following treatment; (iv) reduction in the size and number of DFUs, which can be assessed up to 24-months following treatment; (v) improvement in transcutaneous oxygen level, which can be assessed at six months following treatment; (vi) improvement in pulse volume recording, which can be assessed at six months following treatment; (vii) time to major amputation, which can be assessed up to 24-months following treatment; (viii) improvement on the Wagner Grading Scale, which can be assessed up to 24-months following treatment; (ix) improvement in Rutherford criteria, which can be assessed at six months following treatment; and (x) improvement in leg rest pain score, which can be assessed up to 24-months following treatment; and (xi) improvement in quality of life of the subject as assessed by (i) a 36-item Short Form Health Survey (SF-36) (see, e.g., Ware et al., Medical Care 30(6):473-483); (ii) the Diabetic Foot Ulcer Scale Short Form (DFS-SF) (see, e.g., Bann et al., Pharmacoeconomics, 2003, 21(17):1277-90); (iii) the Patient Global Impression of Change Scale (see, e.g., Kamper et al., J. Man. Manip. Ther., 2009, 17(3):163-170); and/or (iv) the EuroQol5D (EQ-5D™) Scale.

Subject Selection

The following eligibility criteria may be used to select subjects for whom treatment with IL-1β-stimulated CD10⁺, CD34⁻, CD105⁺, CD200⁺ placental stem cells is considered appropriate. All relevant medical and non-medical conditions are taken into consideration when deciding whether this treatment protocol is suitable for a particular subject.

Subjects should meet the following conditions to be eligible for the treatment protocol:
Males and females, at least 18 years of age or older.
Understand and voluntarily sign an informed consent document prior to any study related assessments/procedures are conducted.
Able to adhere to the study visit schedule and other protocol requirements.
Diabetes mellitus Type 1 or Type 2.
Diabetic foot ulcer with severity of Grade 1 (full thickness only) or Grade 2 on the Wagner Grading Scale of greater than one month duration which has not adequately responded to conventional ulcer therapy with a size of at least of 1 cm² except if present on the toe. The maximum lesion size range in the index ulcer is ≤6.25 cm². The measurement of the index ulcer is to be evaluated and measured after debridement (if necessary) at the Screening Visit.
Subjects that meet one or more of the following criteria of arterial insufficiency in the foot with the index ulcer:
   a. Peripheral arterial disease with ABI≥0.4 and ≤0.8 or TBI≥0.30 and ≤0.65.
   b. Transcutaneous oxygen (TcPO2) measurement between 20-40 mmHg. The area measured with TcPO2 should be free of edema and thickened skin.
No planned revascularization or amputation over the next 3 months after screening visit.
Screening should not begin until at least 14 days after a failed reperfusion intervention and at least 30 days after a successful reperfusion intervention.
Subjects should be receiving appropriate medical therapy for hypertension and diabetes and any other chronic medical conditions for which they require ongoing care.
A female of childbearing potential (FCBP) must have a negative serum pregnancy test at Screening and a negative urine pregnancy test prior to treatment with study therapy. In addition, sexually active FCBP must agree to use 2 of the following adequate forms of contraception methods simultaneously such as: oral, injectable, or implantable hormonal contraception; tubal ligation; IUD; barrier contraceptive with spermicide or vasectomized partner for the duration of the study and the Follow-up Period.
Males (including those who have had a vasectomy) must agree to use barrier contraception (latex condoms) when engaging contraception (latex condoms) in reproductive sexual activity with FCBP for the duration of the study and the Follow-up Period

Subjects having one or more of the following conditions can be excluded from the treatment protocol:
Any significant medical condition, laboratory abnormality, or psychiatric illness that would prevent the subject from participating in the study.
Any condition including the presence of laboratory abnormalities, which places the subject at unacceptable risk if he or she were to participate in the study.
Any condition that confounds the ability to interpret data from the study.
Known to be positive for human immunodeficiency virus, Hepatitis C virus, or active infection with Hepatitis B virus.
Pregnant or lactating females.
Subjects with a body mass index >40 at Screening.
AST (SGOT) or ALT (SGPT)>2.5× the upper limit of normal (ULN) at Screening.
Estimated glomerular filtration rate (eGFR)<30 mL/min/1.73 m² at Screening calculated using the Modification of Diet in Renal Disease Study equation (Levey, 2006) or history of eGFR decline >15 mL/min/1.73 m² in the past year.
Alkaline phosphatase >2.5× the ULN at Screening.
Bilirubin level >2 mg/dL (unless subject has known Gilbert's disease) at Screening.
Untreated chronic infection or treatment of any infection with systemic antibiotics, including the ulcer site, must be free of antibiotics within 1 week prior to dosing with IP.
Active osteomyelitis, infection, or cellulitis at or adjacent to the index ulcer.
Index ulcer that has decreased or increased in size by ≥30% during the Screening/Run-In Period.
Pain at rest due to limb ischemia.
Transcutaneous oxygen measurements ≤20 mmHg in the foot with the index ulcer.
Heel ulcers.
Uncontrolled hypertension (defined as diastolic blood pressure >100 mmHg or systolic blood pressure >180 mmHg during Screening at 2 independent measurements taken while subject is sitting and resting for at least 5 minutes).
Poorly controlled diabetes mellitus (hemoglobin A1c>12% or a screening serum glucose of ≥300 mg/dl).
Untreated proliferative retinopathy.
History of malignant ventricular arrhythmia, CCS Class III-IV angina pectoris, myocardial infarction/percutaneous coronary intervention (PCI)/coronary artery bypass graft (CABG) in the preceding 6 months prior to signing the informed consent form (ICF), pending coronary revascularization in the following 3 months, transient ischemic attack/cerebrovascular accident in the preceding 6 months, prior to signing the ICF, and/or New York Heart Association [NYHA] Stage III or IV congestive heart failure.
Abnormal ECG: new right bundle branch block (BBB)≥120 msec in the preceding 3 months prior to signing the ICF.
Uncontrolled hypercoagulation.
Life expectancy less than 2 years at the time of signing the ICF due to concomitant illnesses.
In the opinion of the Investigator, the subject is unsuitable for cellular therapy.
History of malignancy within 5 years prior to signing the ICF except basal cell or squamous cell carcinoma of the skin or remote history of cancer now considered cured or positive Pap smear with subsequent negative follow-up.
History of hypersensitivity to any of the components of the product formulation (including bovine or porcine products, dextran 40, and dimethyl sulfoxide [DMSO]).
Subject has received an investigational agent an agent or device not approved by the US Food and Drug Administration (FDA) for marketed use in any indication—within 90 days (or 5 half-lives, whichever is longer) prior to treatment with study therapy or planned participation in another therapeutic study prior to the completion of this study.
Subject has received previous investigational gene or cell therapy.

Clinical Outcome

Efficacy of the IL-1β-stimulated CD10⁺, CD34⁻, CD105⁺, CD200⁺ placental stem cells in treatment of DFU is confirmed if improvement in one or more clinical endpoints is demonstrated.

### 6.7 Example 7: Alternate DFU Treatment Protocol

Subjects having diabetic foot ulcer (DFU) with peripheral arterial disease (PAD), at least 18 years of age, are treated with IL-1β-stimulated CD10⁺, CD34⁻, CD105⁺, CD200⁺ placental stem cells. Subject Group I: 3×10⁶ IL-1-stimulated CD10⁺, CD34⁻, CD105⁺, CD200⁺ placental stem cells are administered intramuscularly on days 1 (the first day of treatment), 29, and 57. Subject Group II: 3×10⁷ IL-1β-stimulated CD10⁺, CD34⁻, CD105⁺, CD200⁺ placental stem cells are administered intramuscularly on days 1 (the first day of treatment), 29, and 57. Subject Group III: placebo is administered intramuscularly on days 1 (the first day of treatment), 29, and 57.

Clinical Endpoints

A primary clinical endpoint for efficacy of IL-1β-stimulated CD10⁺, CD34⁻, CD105⁺, CD200⁺ placental stem cells for treating DFU can be improvement in limb vascular function as assessed by measurement of ankle brachial index (ABI); transcutaneous oximetry (TCOM), near infrared spectroscopy, Fludeoxyglucose positron emission tomography/computed tomography (FGD PET/CT), Doppler ultrasound, magnetic resonance imaging (MRI), angiography, and/or oximetry. Improvement in limb vascular function can be assessed at approximately one year following treatment.

Other clinical endpoints for efficacy of IL-1-stimulated CD10⁺, CD34⁻, CD105⁺, CD200⁺ placental stem cells for treating DFU can include: (i) ulcer closure and complete wound closure of the index ulcer (ulcer closure can be represented by skin closure without drainage or need for dressing; complete closure can be represented by retention of ulcer closure for at least four weeks following determination of closure), which can be assessed at approximately one year following treatment; (ii) reduction of the frequency and severity of adverse events, which can be assessed at approximately one year following treatment; (iii) reduction in the number, size of all ulcers and 50% closure of the index ulcer, which can be assessed at approximately one year following treatment; (iv) a reduction in time to major amputation of the treated leg, which can be assessed at approximately one year following treatment; (v) improvement on the Wagner Grading Scale, which can be assessed at approximately one year following treatment; (vi) improvement in Rutherford criteria, which can be assessed at approximately one year following treatment; (vii) improvement in leg rest pain score, which can be assessed at approximately one year following treatment; and (viii) improvement in quality of life of the subject as assessed using the Patient Global Impression of Change in Neuropathy (PGICN).

Subject Selection

The following eligibility criteria may be used to select subjects for whom treatment with IL-1β-stimulated CD10⁺, CD34⁻, CD105⁺, CD200⁺ placental stem cells is considered appropriate. All relevant medical and non-medical conditions are taken into consideration when deciding whether this treatment protocol is suitable for a particular subject.

Subjects should meet the following conditions to be eligible for the treatment protocol:
Males and females, at least 18 years of age or older.
Diabetes mellitus Type 1 or Type 2.
Diabetic foot ulcer with severity of Grade 1 (full thickness only) or Grade 2 on the Wagner Grading Scale of greater than one month duration which has not adequately responded to conventional ulcer therapy.
Subjects that meet one or more of the following criteria of arterial insufficiency in the foot with the index ulcer:
   a. Peripheral arterial disease with ABI≥0.4 and ≤0.8 or TBI≥0.30 and ≤0.65.
   b. Transcutaneous oxygen (TcPO2) measurement between 20-40 mmHg.
No planned revascularization or amputation over the next 3 months after screening visit.
Dosing should not begin until at least 14 days after a failed reperfusion intervention and at least 30 days after a successful reperfusion intervention.

Subjects having one or more of the following conditions can be excluded from the treatment protocol:
Any significant medical condition, laboratory abnormality, or psychiatric illness that would prevent the subject from participating in the study.
Any condition including the presence of laboratory abnormalities, which places the subject at unacceptable risk if he or she were to participate in the study.
Pregnant or lactating females.
Subjects with a body mass index >40 at Screening.
Estimated glomerular filtration rate (eGFR)<30 mL/min/1.73 m² at Screening calculated using the Modification of Diet in Renal Disease Study equation (Levey, 2006) or history of eGFR decline >15 mL/min/1.73 m² in the past year.
Untreated chronic infection or treatment of any infection with systemic antibiotics, including the ulcer site, must be free of antibiotics within 1 week prior to dosing with IP.
Known osteomyelitis, infection, or cellulitis at or adjacent to the index ulcer.
Limb pain at rest due to limb ischemia.
Uncontrolled hypertension (defined as diastolic blood pressure >100 mmHg or systolic blood pressure >180 mmHg during Screening at 2 independent measurements taken while subject is sitting and resting for at least 5 minutes).
Poorly controlled diabetes mellitus (hemoglobin A1c >12% or a screening serum glucose of ≥300 mg/dl).
Untreated proliferative retinopathy.
History of malignant ventricular arrhythmia, CCS Class III-IV angina pectoris, myocardial infarction/percutaneous coronary intervention (PCI)/coronary artery bypass graft (CABG) in the preceding 6 months prior to signing the informed consent form (ICF), pending coronary revascularization in the following 3 months, transient ischemic attack/cerebrovascular accident in the preceding 6 months, prior to signing the ICF, and/or New York Heart Association [NYHA] Stage III or IV congestive heart failure.
Abnormal ECG: new right bundle branch block (BBB)≥120 msec in the preceding 3 months prior to signing the ICF.
Uncontrolled hypercoagulation.
Life expectancy less than 2 years at the time of signing the ICF due to concomitant illnesses.
In the opinion of the Investigator, the subject is unsuitable for cellular therapy.
History of malignancy within 5 years prior to signing the ICF except basal cell or squamous cell carcinoma of the skin or remote history of cancer now considered cured or positive Pap smear with subsequent negative follow-up.
History of hypersensitivity to any of the components of the product formulation (including bovine or porcine products, dextran 40, and dimethyl sulfoxide [DMSO]).
Subject has received an investigational agent an agent or device not approved by the US Food and Drug Administration (FDA) for marketed use in any indication—within 90 days (or 5 half-lives, whichever is longer) prior to treatment with study therapy or planned participation in another therapeutic study prior to the completion of this study.
Subject has received previous investigational gene or cell therapy.

Clinical Outcome

Efficacy of the IL-1β-stimulated CD10⁺, CD34⁻, CD105⁺, CD200⁺ placental stem cells in treatment of DFU is confirmed if improvement in one or more clinical endpoints is demonstrated.

1. An isolated stimulated placental derived adherent cell, wherein said cell is adherent to tissue culture plastic, and wherein said cell is CD10⁺, CD34⁻, CD105⁺ and CD200⁺, wherein said cell has been contacted with one or more pro-inflammatory cytokines in vitro, and wherein said cell (1) promotes the proliferation of endothelial cells; (2) promotes the formation of sprouts or tube-like structures in a population of endothelial cells; or (3) promotes the migration of endothelial cells.

2. (canceled)

3. The isolated cell of claim 1, wherein said pro-inflammatory cytokine is one or more of IL-1α, IL-1β, IL-6, IL-8, IL-18, TNF-α, or INF-γ.

4. (canceled)

5. The isolated cell of claim 1, wherein said cell secretes trophic factors at a higher level than an isolated placental derived adherent cell that has not been contacted with said one or more pro-inflammatory cytokines.

6. The isolated cell of claim 5, wherein said trophic factors comprise at least one of GM-CSF, G-CSF, IL-6, GRO, MCP-1, Follistatin, or IL-8.

7. (canceled)

8. An isolated population of cells comprising a cell of claim 1.

9. The isolated population of cells of claim 8, wherein at least 50% of the cells in said population are the cells of claim 1.

10. (canceled)

11. A method of treating an individual having a disease or disorder of the circulatory system, comprising administering a population of the cells of claim 1 to said individual.

12. (canceled)

13. The method of claim 11, wherein said disease or disorder is diabetic foot ulcer (DFU).

14. The method of claim 13, wherein said DFU is caused by and/or associated with peripheral arterial disease (PAD).

15. The method of claim 11, wherein said disease or disorder of the circulatory system is a heart disease or injury.

16. The method of claim 11, wherein said disease or disorder is a disruption of blood flow in or around a limb.

17. (canceled)

18. (canceled)

19. (canceled)

20. (canceled)

21. (canceled)

22. The method of claim 11, wherein one or more indicia of cardiac function is detectibly improved, wherein said indicia of cardiac function are chest cardiac output (CO), cardiac index (CI), pulmonary artery wedge pressure (PAWP), cardiac index (CI), % fractional shortening (% FS), ejection fraction (EF), left ventricular ejection fraction (LVEF); left ventricular end diastolic diameter (LVEDD), left ventricular end systolic diameter (LVESD), contractility (dP/dt), a decrease in atrial or ventricular functioning, an increase in pumping efficiency, a decrease in the rate of loss of pumping efficiency, a decrease in loss of hemodynamic functioning, or decrease in complications associated with cardiomyopathy, as compared to the individual prior to administration of placental derived adherent cells.

23. (canceled)

24. The method of claim 23, wherein the population of cells is administered to said individual in an amount and for a time sufficient for detectable improvement of one or more said indicia of cardiac function.

25. The method of claim 23, wherein said disease or disorder of the circulatory system is a heart disease or injury.

26. A method of treating an individual having a disruption of blood flow in or around a limb, comprising administering a therapeutically effective amount of a population of the cells of claim 1.

27. The method of claim 26, wherein said disruption of blood flow in or around the limb is a peripheral arterial disease (PAD) or a peripheral vascular disease (PVD).

28. The method of claim 11, wherein the method further comprises administering a further therapeutic agent.

29. The method of claim 11, wherein said administration is by transplantation, implantation, injection, infusion, or delivery via catheter.

30. A method of producing a stimulated placental derived adherent cell, wherein said cell is CD10⁺, CD34⁻, CD105⁺ and CD200⁺, the method comprising contacting a placental derived adherent cell with one or more pro-inflammatory cytokines in vitro.

31. The method of claim 30, wherein the pro-inflammatory cytokine is one or more of IL-1α, IL-1β, IL-6, IL-8, IL-18, TNF-α, or INF-γ.

32. (canceled)

33. (canceled)

34. (canceled)
